(19) United States
(12) Patent Application Publication
(10) Pub. No.: US 2024/0016391 A1
(43) Pub. Date: Jan. 18, 2024

(54) APPARATUS FOR DIAGNOSING AND/OR TREATING MALARIA

(71) Applicant: Masimo Corporation, Irvine, CA (US)

(72) Inventors: Dmitri O. Lapotko, Dana Point, CA (US); Katsiaryna Hleb, Irvine, CA (US); Mohamed K. Diab, Ladera Ranch, CA (US)

(21) Appl. No.: 18/472,047

(22) Filed: Sep. 21, 2023

Related U.S. Application Data
(63) Continuation of application No. 16/213,923, filed on Dec. 7, 2018, now abandoned.
(60) Provisional application No. 62/666,011, filed on May 2, 2018.
(60) Provisional application No. 62/595,971, filed on Dec. 7, 2017.

(51) Int. Cl. A61B 5/00

(52) U.S. Cl. CPC A61B 5/0095

(57) ABSTRACT

A malaria diagnosis and/or treatment apparatus can include an optical source and an acoustic detector in a single probe (sensor). The optical source can provide optical energy configured to produce transient vapor nanobubbles around malaria-specific nanoparticles, such as hemozoin in skin, blood and other tissues infected with malaria, but not in uninfected tissues. The acoustic detector can detect pressure pulses generated by the transient vapor nanobubbles. A malaria diagnosis and/or screening process can be based on using several metrics of the detector signal output, which include the time and amplitude parameters of such signal. This metrics characterize both active and residual forms of malaria disease and can be used in the clinical diagnostics of malaria and in mass screening of the malaria transmission.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. Specifically, this application claims the priority benefit of U.S. Nonprovisional application Ser. No. 16/213,923, filed Dec. 7, 2018, U.S. Provisional Application No. 62/595,971, filed Dec. 7, 2017, and U.S. Provisional Application No. 62/666,011, filed May 2, 2018, the entirety of each of which is hereby incorporated by reference and should be considered a part of this specification.

### FIELD

The present application relates generally to the fields of detection and elimination of malaria parasites in a patient's body, in particular, with the use of laser-induced transient vapor nanobubbles.

### BACKGROUND

Malaria is a widespread and infectious disease that can cause serious illness and death in humans. A patient can be infected when a malaria parasite infects cells of the patient, also known as a host. The parasite can produce hemozoin (HZ), which are nanocrystals formed when the parasite digests the hemoglobin in the host's red blood cells. Malaria-infected red blood cells or other body tissue infected by malaria parasites contain HZ (hemozoin) nanocrystals.

Current malaria diagnosis techniques include, for example, rapid diagnostic tests (RDTs), microscopy, and polymerase chain reaction (PCR). These diagnosis techniques analyze a patient's blood samples. RDT analyzes the proteins in the blood to look for presence of malaria parasites and is approved by the World Health Organization (WHO). Microscopy uses stain of a thick blood slide, such as with a 200 to 500 white blood cell count, to determine malaria parasite density and gametocyte counts. Microscopy is also WHO-approved for malaria diagnosis. PCR analyzes DNAs in the blood to determine presence of malaria parasites.

Malaria can be treated and/or prevented by administration of antimalarial drugs, such as quinine, chloroquine, atovaquone/proguanil, and others.

### SUMMARY

Current malaria diagnosis generally employ invasive techniques which are costly, time-consuming and have low accuracy. The diagnosis and treatment of malaria can require separate procedures.

Antimalarial drugs have several disadvantages. Malaria parasites can develop drug resistance to the antimalarial drugs. The drugs can also be ineffective against malaria parasites that escape from the blood vessels into the tissue and/or skin of the patient through micro-capillaries in the tissue, also known as tissue-sequestered parasites. Tissue-sequestered parasites can cause lethal complications in the patient after treatments with antimalarial drugs, such as when the patient has low blood levels of parasites, and/or can cause relapses in patients treated with antimalarial drugs. Current malaria diagnosis techniques, such as RDTs, microscopy, and PCR, are not able to detect tissue-sequestered malaria parasites as these techniques rely on analyzing the patient's peripheral blood samples.

Current malaria diagnosis techniques also may not detect the HZ (hemozoin) nanocrystals without an active and/or live malaria parasite. However, detecting the HZ (hemozoin) nanocrystals without an active or live malaria parasite can provide valuable information for determining recent or past presence of malaria infection, the data important in screening and understanding the malaria transmission.

Laser-induced transient vapor nanobubbles can be used to diagnose and/or treat malaria in a noninvasive, efficient, and reproducible manner. The transient vapor nanobubbles can be generated around one or more malaria-specific nanoparticles (such as one or more HZ (hemozoin) nanocrystals (with or without an active malaria parasite) or malaria-specific nanoparticles introduced into the host red blood cells) when laser pulses are applied to the nanoparticles. The laser pulses can cause rapid heating of the malaria-specific nanoparticles, but not of uninfected red blood cells or other host tissues. Liquid (such as water) around the malaria-specific nanoparticles can rapidly evaporate, leading to the generation of a transient vapor nanobubble. The generation of transient vapor nanobubbles can be detected by acoustic detectors. In some embodiments, the transient nanobubble-based malaria detection mechanism can detect a single hemozoin nanoparticle. The transient nanobubble-based malaria detection mechanism disclosed herein can be advantageous over the bulk photoacoustic mechanism, which requires a large number of hemozoin nanoparticles to produce a detectable malaria-positive signal.

As the transient vapor nanobubble size increases with increasing energy level of the laser pulses, in some instances, the energy level of the laser pulses can be high enough to generate transient vapor nanobubbles that can cause mechanical damage to the HZ (hemozoin) nanocrystal host, the malaria parasite, the malaria-infected red blood cell, or a combination thereof. Additional details of employing transient vapor nanobubbles to detect and/or treat malaria-infected red blood cells are described in International Application No. WO2013/109722, filed Jan. 17, 2013 and titled “Theranostic methods and systems for diagnosis and treatment of malaria,” attached as Appendix A, the entirety of which is incorporated herein by reference and should be considered a part of the specification.

The vapor nanobubbles, also referred to as HZ (hemozoin)-generated vapor nanobubbles (HVNB) or nanobubbles, can be generated on a liquid sample test (such as blood, in particular peripheral blood, or urine), or on a patient's skin using a sensor that optically excites the malaria-specific nanoparticle in the skin to generate hemozoin-generated vapor nanobubbles.

In order to use transient vapor nanobubbles for detecting and/or treating malaria noninvasively, the laser pulses must penetrate a patient's skin and reach the malaria-specific nanoparticles despite attenuation of the laser pulses by the patient's body tissue as the laser pulses travel deeper under the skin. The optical delivery part of the malaria sensor, such as an optical fiber, needs to be brought as close as possible to the malaria-specific nanoparticles or malaria parasite. The nanobubble-generated pressure pulses reaching the surface of the acoustic detector also need to be strong enough for an acoustic (ultrasound) signal of that pressure pulse to be detected. Challenges in improving the sensitivity and/or specificity of the acoustic detector can include reducing a distance between the malaria-specific nanoparticles and the acoustic detector, and/or reducing a distance between an optical source and the acoustic detector.

In some instances, a malaria diagnosis and/or treatment apparatus can combine an optical source and an acoustic detector in a single probe (also referred to as a malaria sensor). The single probe can bring the acoustic detector closer to the source of the acoustic pulse, which is the transient vapor nanobubble, than having separate optical source and acoustic detector probes. However, the distance between the acoustic detector surface and the malaria-specific nanoparticle in the known single probes can still be too large. This can be due to the use of a spherical acoustic detector for detecting the transient vapor nanobubble, as the transient vapor nanobubble is considered a point source. The spherical acoustic detector can also have a large surface area, which weakens the signal output from the spherical detector. Other concerns with using a spherical acoustic detector can include high cost and structural complexity, which can make it infeasible to mass-produce the single probe.

The malaria probe according to the present disclosure can include the optical source and the acoustic detector in a single probe, and can diagnose and/or treat malaria. The malaria probe according to the present disclosure can increase the sensitivity and specificity of the malaria detection by having one or more small and substantially flat acoustic detectors placed in close proximity to the optical source, which can include one or more optical fibers, and to a probe tip surface. Sensitivity can be indicative of the probe's ability to correctly detect malaria-positive cases. Specificity can be indicative of the probe's ability to avoid false positive and false negative detections. In some embodiments, the malaria probe is able to detect tissue-sequestered malaria parasites. Embodiments of the malaria probe can also be immune to resistance from the malaria parasite, efficient, and/or safe for the patient. In some embodiments, the malaria probe can be cheap to build and/or economically feasible for mass production.

An apparatus configured for diagnosing malaria noninvasively can comprise: a sensor probe having a probe body terminating at a probe tip surface, the probe tip surface configured to be placed into contact with a predetermined detection location, the predetermined detection location being in vivo on a patient's skin or ex vivo in a patient's body fluid sample; an optical source configured to generate a plurality of laser pulses of at least one predetermined energy level or at least one predetermined wavelength, the optical source terminating at or near the probe tip surface, the laser pulses configured to cause generation of one or more transient vapor nanobubbles around malaria-specific nanoparticles at the predetermined detection location; and one or more acoustic detectors configured to detect acoustic pulses generated by the one or more transient vapor nanobubbles and output one or more signals indicative of the detected acoustic pulses to at least one processor, the one or more acoustic detectors comprising a piezo element and being flat, wherein a distance between an outer wall of the optical source and a radially inner edge of the one or more acoustic detectors, R1, can be 0.01 mm to 0.03 mm so as to improve a signal strength of the acoustic pulses striking a flat surface of the one or more acoustic detectors, wherein the R1 can be 0.01 mm to 0.03 mm such that the acoustic pulses strike the flat surface of the one or more acoustic detectors at an angle of incidence, a, of less than 45°, wherein the optical source and the one or more acoustic detectors can be enclosed within the probe body.

In a configuration, the optical source can comprise one or more optical fibers.

In a configuration, the one or more optical fibers can each have a core diameter of about 100 μm.

In a configuration, the piezo element can comprise a navy type II or type VI material or a composite material.

In a configuration, a tissue-facing surface of the one or more acoustic detectors can be 0.1 mm to 0.3 mm recessed from the probe tip surface.

In a configuration, the apparatus can further comprise a front layer between the probe tip surface and a tissue-facing surface of the one or more acoustic detectors.

In a configuration, an outer surface of the one or more optical fibers can be separated from a radially outer edge of the one or more acoustic detectors by 0.3 mm to 1.5 mm.

In a configuration, the malaria-specific nanoparticles can be located within an optical penetration depth beneath the predetermination location.

In a configuration, an outer diameter of the one or more acoustic detectors can be 0.2 mm to 3 mm such that the acoustic pulses strike the flat surface of the one or more acoustic detectors at a same or similar angle of incidence to reduce an effect of de-phasing.

An apparatus configured for diagnosing malaria noninvasively can comprise: a sensor portion, the sensor portion including an optical source configured to generate laser pulses of at least one predetermined energy level, the optical source comprising an optical fiber terminating at or near a distal end of the sensor portion, the laser pulses configured to cause generation of one or more nanobubbles around malaria-specific nanoparticles at a predetermined location, and one or more acoustic detectors configured to detect acoustic pulses generated by the one or more nanobubbles and output one or more signals indicative of the detected acoustic pulses to at least one processor, the one or more acoustic detectors comprising a piezo element and being flat, wherein, at the distal end of the sensor portion, a distance between an outer wall of the optical fiber and a radially inner edge of the one or more acoustic detectors, R1, can be 0.01 mm to 0.03 mm so as to improve a signal strength of the acoustic pulses striking a flat surface of the one or more acoustic detectors, wherein the R1 can be 0.01 mm to 0.03 mm such that the acoustic pulses strike the flat surface of the one or more acoustic detectors at an angle of incidence, a, of less than 45°; a housing, wherein the sensor portion can be at least partially disposed within the housing; and a spring disposed between a proximal end of the housing and the proximal end of the sensor portion, the spring biasing the sensor portion toward a distal end of the housing.

In a configuration, the spring can be configured to be compressed when the apparatus is applied to a measurement site, the compressed spring forcing the distal end of the sensor portion into contact with the measurement site.

In a configuration, the housing can comprise a patient interface at the distal end, the apparatus further comprising a liner covering the patient interface when the apparatus is not in use.

In a configuration, the patient interface can comprise an adhesive layer and/or a gel layer.

In a configuration, an outer diameter of the one or more acoustic detectors can be 0.2 mm to 3 mm such that the acoustic pulses strike the flat surface of the one or more acoustic detectors at a same or similar angle of incidence to reduce an effect of de-phasing.

An apparatus for diagnosing and/or treating malaria in a patient noninvasively can comprise a sensor probe having a probe body terminating at a probe tip surface, the probe tip surface configured to be placed into contact with a predetermined detection location; an optical source configured to generate a plurality of laser pulses of at least one predetermined energy level and/or at least one predetermined wavelength, the optical source terminating at or near the probe tip surface, the laser pulses configured to cause generation of one or more transient vapor nanobubbles around malaria-specific nanoparticles at the predetermined location; and one or more acoustic detectors configured to detect acoustic pulses generated by the one or more transient vapor nanobubbles and output one or more signals indicative of the detected acoustic pulses to at least one processor, the one or more acoustic detectors being substantially flat and in close proximity with the optical source, wherein the optical source and the acoustic detector can be enclosed within the probe body. The optical source can comprise one or more optical fibers. The apparatus can comprise two or more optical fibers, wherein each of the two or more optical fibers can be located between two acoustic detectors The optical fiber can have a core diameter of about 50 μm to about 200 μm, or about 100 μm. The optical source can further comprise a laser pulse generator coupled to the one or more optical fibers. The acoustic detector can comprise a piezo element. The apparatus can comprise two or more piezo elements configured to detect signals of the same or different frequency spectra. The piezo element can comprise a navy type II or type VI material, or a composite material. The acoustic detector can comprise a substantially centrally located opening sized to accommodate the optical fiber. The acoustic detector can comprise a substantially flat disc, or two or more substantially flat discs or elements of other shape and acoustic properties. A tissue-facing surface of the acoustic detector can be about 0.1 mm to about 0.3 mm recessed from the probe tip surface. The sensor probe can further comprise a front layer between the probe tip surface and a tissue-facing surface of the acoustic detector. An outer wall of the optical fiber can be separated from a radially inner edge of the acoustic detector by about 0.01 mm to about 0.03 mm. An outer surface of the optical source can be separated from a radially outer edge of the acoustic detector by about 1.0 mm to about 1.5 mm. The sensor probe can further comprise a disposable cap. The laser pulses can be configured to cause generation of transient vapor nanobubbles around malaria-specific nanoparticles in blood and/or tissue. The predetermined detection location can be a patient's skin at the patient's wrist, ankle, lip, or tongue base or other locations. The predetermined detection location can be a surface of a flow cuvette with a flow path for a patient's blood or urine or other biological fluid sample. The probe tip surface can be configured to be covered with a layer of gel before being placed into contact with the predetermined detection location. The malaria-specific nanoparticles are located within the optical penetration depth beneath such location. The apparatus can further comprise a housing, wherein the probe body can be at least partially disposed within the housing; and a spring disposed between a proximal end of the housing and the proximal end of the probe body, the spring biasing the probe body toward a distal end of the housing.

An apparatus configured for diagnosing and/or treating malaria noninvasively can comprise a sensor portion, the sensor portion including: an optical source configured to generate laser pulses of at least one predetermined energy level, the optical source comprising an optical fiber terminating at or near a distal end of the sensor portion, the laser pulses configured to cause generation of one or more transient vapor nanobubbles around malaria-specific nanoparticles at the predetermined location; and one or more acoustic detectors configured to detect acoustic pulses generated by the one or more transient vapor nanobubbles and output one or more signals indicative of the detected acoustic pulses to at least one processor, the acoustic detector being substantially flat and in close proximity with the optical fiber at the distal end of the sensor portion; a housing, wherein the sensor portion can be at least partially disposed within the housing; and a spring disposed between a proximal end of the housing and the proximal end of the sensor portion, the spring biasing the sensor portion toward a distal end of the housing. The spring can be configured to be compressed when the apparatus is applied to a measurement site, the compressed spring forcing the distal end of the sensor portion into contact with the measurement site. The housing can comprise a patient interface at the distal end, the apparatus further comprising a liner substantially covering the patient interface when the apparatus is not in use. The patient interface can comprise an adhesive layer and/or gel layer.

A method of detecting malaria using any of the apparatuses disclosed herein can comprise instructing a laser pulse source to apply one or more laser pulses to a measurement site, any of the apparatuses disclosed herein being applied to the measurement site; receiving one or more signals from the acoustic detector of the apparatus, the one or more signals indicative of acoustic pulses detected by the acoustic detector upon the application of the one or more laser pulses to the measurement site; determining whether the measurement site is malaria-positive by: determining electronically a peak time of the one or more signals; comparing the peak time with a predetermined diagnostic threshold; and outputting a malaria-positive message if the peak time exceeds the predetermined diagnostic threshold, and outputting a malaria-negative message if the peak time does not exceed the predetermined diagnostic threshold. Determining whether the measurement site is malaria-positive can further comprise determining parameters from an amplitude, phase and/or shape of the signal. The method can also include using any of the apparatuses disclosed herein to scan a plurality of close locations to probe a sufficient volume of skin so as to improve detection of low level of malaria parasite density in the skin.

A method of detecting malaria using any of the apparatuses disclosed herein can comprise instructing a laser pulse generator to apply one or more laser pulses to a measurement site, the apparatus being applied to the measurement site; receiving one or more signals from the one or more acoustic detectors of the apparatus, the one or more signals indicative of acoustic pulses detected by the acoustic detector upon the application of the one or more laser pulses to the measurement site; and determining whether the measurement site is malaria-positive based on parameters from an amplitude, phase, shape, and/or a peak time delay of the one or more signals. Instructing can comprise instructing a laser pulse generator to apply one or more laser pulses of the same or different energy levels and/or wavelengths. Instructing can comprise instructing a laser pulse generator to route the laser pulses sequentially to a plurality of optical fibers. Receiving can comprise receiving a signal from a high-frequency one of the one or more acoustic detectors and a signal from a low-frequency one of the one or more acoustic detectors.

### BRIEF DESCRIPTION OF THE DRAWINGS

Various embodiments are depicted in the accompanying drawings for illustrative purposes and may not be drawn to scale, and should in no way be interpreted as limiting the scope of the embodiments. In addition, various features of different disclosed embodiments can be combined to form additional embodiments, which are part of this disclosure. In the drawings, similar elements have reference numerals with the same last two digits.

FIGS. 1A and 1B illustrate schematically example systems for diagnosing and/or treating malaria using laser-induced transient vapor nanobubbles.

FIG. 2 illustrates an example flow chart of malaria diagnosis and/or treatment using laser-induced transient vapor nanobubbles.

FIGS. 3A-3D illustrate an example malaria probe according to some embodiments of the present disclosure.

FIG. 3E illustrates a perspective view of a sensor portion of the malaria probe of FIGS. 3A-3C.

FIG. 3F illustrates a partial side view of a distal sensor portion of FIG. 3E.

FIG. 4A illustrates schematically a longitudinal cross-section of a distal portion of a known malaria probe having an optical source and a spherical acoustic detector.

FIG. 4B illustrates schematically spherical wavefronts arriving at different portions of a flat acoustic detector.

FIG. 5A illustrates schematically a longitudinal cross-section of a distal portion of an example malaria probe having an optical source and a flat acoustic detector.

FIG. 5B illustrates a longitudinal cross-section of the distal sensor portion of FIG. 3E along the axis 5B-5B.

FIG. 5C illustrates a transverse cross-section of the distal sensor portion of FIG. 3E along the axis 5C-5C.

FIG. 5D illustrates a transverse cross-section of the distal sensor portion of FIG. 5B along the axis 5D-5D.

FIG. 5E illustrates a longitudinal cross-section of a fiber housing in the sensor portion of FIG. 3D.

FIG. 5F illustrates an example optical fiber of the sensor portion of FIG. 3D.

FIG. 5G illustrates a longitudinal cross-section of a support body in the sensor portion of FIG. 3D.

FIG. 6A illustrates schematically a comparison of the two acoustic detectors located at different distances from the malaria parasite and having two different angles of incidence of a pressure pulse.

FIG. 6B illustrates schematically a comparison of the spherical acoustic detector of FIG. 3A and the flat acoustic detector of FIG. 4.

FIG. 7A illustrates schematically certain dimensions of an optical source and a flat acoustic detector in an example malaria probe when viewed from a side of the probe.

FIG. 7B illustrates schematically dimensions of an optical source and a flat acoustic detector in an example malaria probe when viewed from an end of the probe.

FIG. 7C illustrates schematically an example malaria probe applied to tissue with a subcutaneous malaria-specific nanoparticle.

FIG. 7D illustrates schematically a malaria sensor probe having an optical fiber and two flat acoustic detectors.

FIG. 7E illustrates schematically a malaria sensor probe having a plurality of optical fiber-acoustic detector combinations.

FIGS. 8A-8C illustrate schematically various example locations of malaria parasite in a host.

FIGS. 9A-9C illustrate example flow cuvettes configured to be used with a malaria probe.

FIG. 10A illustrates schematically a perspective view of an example malaria probe having an optical source and a flat acoustic detector.

FIG. 10B illustrates schematically a cross-sectional view of the malaria probe of FIG. 10A.

FIG. 10C illustrates schematically a detailed view of a tip portion of the malaria probe of FIG. 10B.

FIG. 11A illustrates schematically a top view of the malaria probe of FIG. 10A.

FIG. 11B illustrates schematically a side view of the malaria probe of FIG. 10A.

FIG. 11C illustrates schematically a cross-sectional view of the malaria probe of FIG. 11A.

FIG. 11D illustrates schematically a detailed view of the tip portion of the malaria probe of FIG. 11C.

FIG. 12A illustrates schematically a perspective view of the tip portion of the malaria probe of FIG. 10A.

FIG. 12B illustrates schematically a cross-sectional view of the tip portion of the malaria probe of FIG. 12A.

FIG. 13 illustrates schematically a front view of the tip portion of the malaria probe of FIG. 10A.

FIGS. 14A-14D illustrate an example of a spring-loaded malaria probe having an optical source and an acoustic detector.

FIG. 15A illustrates schematically generation of background acoustic pulses in healthy tissues by a laser pulse.

FIG. 15B illustrates schematically generation of acoustic pulses in malaria-infected tissues by a laser pulse.

FIG. 15C illustrates an example flow chart of a malaria detection process based at least in part on signal peak time-delay.

FIG. 16 illustrates an example comparison of acoustic signals from healthy tissues and malaria-infected tissues upon application of laser impulse(s).

FIGS. 17A-17C illustrate signal peak timing-derived time histograms based on studies of a plurality of patient groups.

FIG. 18A illustrates an example signal amplitude-derived parameters of non-invasive skin signals obtained from healthy and malaria-positive human subjects when applying laser pulse(s) to an ankle in the blinded study, by using microscopy-determined malaria status and pre-determined diagnostic threshold.

FIG. 18B illustrates an example signal amplitude-derived parameters of non-invasive skin signals obtained from healthy and malaria-positive human subjects when applying laser pulse(s) to a hand in the blinded study, by using microscopy-determined malaria status and pre-determined diagnostic threshold.

FIG. 18C illustrates an example signal amplitude-derived parameters of blood signals obtained from healthy and malaria-positive human subjects when applying laser pulse(s) to a blood sample in the blinded study, by using microscopy-determined malaria status and pre-determined diagnostic threshold.

FIG. 18D illustrates an example signal amplitude-derived parameters of non-invasive urine signals obtained from healthy and malaria-positive human subjects when applying laser pulse(s) to a urine sample in the blinded study, by using microscopy-determined malaria status and pre-determined diagnostic threshold.

FIGS. 19A-19D illustrate two groups of 60 signals collected from one anatomic location on skin in healthy (19A-19B) and positive (19C-19D) human subjects.

FIG. 19E illustrates an example diagnostic procedure using the malaria probe disclosed herein.

FIGS. 19F and 19G are images of human skin before and after the application of diagnostic laser pulses.

FIG. 20A illustrates an example signal amplitude-derived parameters of non-invasive skin signals obtained from healthy and malaria-positive human subjects when applying laser pulse(s) to an ankle in the blinded human study of Plasmodium Falciparum malaria type, by using microscopy-determined malaria status and pre-determined diagnostic threshold.

FIG. 20B illustrates an example signal amplitude-derived parameters of non-invasive skin signals obtained from healthy and malaria-positive human subjects when applying laser pulse(s) to an ankle in the blinded human study of Plasmodium Vivax malaria type, by using microscopy-determined malaria status and pre-determined diagnostic threshold.

FIG. 20C illustrate the relationship between the skin tone and the malaria diagnostic metrics.

FIGS. 20D and 20E illustrate an example signal amplitude-derived parameters of non-invasive skin signals obtained from asymptomatic randomly chosen human subjects when applying laser pulse(s) to an ankle in the blinded mass screening of asymptomatic cases in human study of Plasmodium Falciparum (20D) and Vivax (20E) malaria types, by using microscopy- and PCR-determined malaria status and pre-determined diagnostic threshold.

FIG. 21A illustrates group-average signal amplitude-derived diagnostic parameters as determined in a clinical application human study for healthy vs malaria-positive subjects.

FIG. 21B illustrates group-average signal amplitude-derived diagnostic parameters as determined in a mass screening human study for malaria-negative vs malaria-positive subjects. There is an apparent difference between signals of healthy (FIG. 21A) and many of malaria-negative (as determined for their peripheral blood with the microscopy method) subjects. This difference suggests some subjects with microscopy-negative peripheral blood may still have parasites in their skin.

FIG. 22A illustrates signal amplitude-derived diagnostic parameters for different malaria parasite densities in human blood model of Plasmodium Falciparum malaria as measured in vitro for 36 hour Plasmodium Falciparum cultured parasites

FIG. 22B illustrates reproducibility of the malaria detection test using signal amplitude-derived diagnostic parameters as in the FIG. 22A.

FIG. 23A illustrates schematically an experimental setup for an experimental model of nanobubble generation in water and skin at the surface of gold nanofilm.

FIGS. 23B and 23C are photos of the experimental setup for the experimental model of nanobubble generation in water and skin at the surface of gold nanofilm.

FIG. 23D illustrates an example spectrum of a hydrophone used with the experimental setup of FIGS. 23A-23C.

FIGS. 23E and 23F illustrate a signal of vapor nanobubble in the gold-water model with typical fist (the nanobubble expansion) and second (nanobubble collapse) spikes.

FIG. 23G illustrates dependence of the first and second spike amplitudes (from a baseline to a maximum) upon the lifetime of a vapor nanobubble generated in water in experimental model of nanobubble generation in water and skin at the surface of gold nanofilm.

FIGS. 23H-J illustrates an example nanobubble-generated acoustic pulse and its metrics, spike amplitude and time interval between first and second spikes, and the energy of the laser pulse used to generate nanobubbles, as detected by a hydrophone-type sensor.

FIG. 23K illustrates a near field signal of a small vapor nanobubble with a hydrophone.

FIG. 24A illustrates an example of the experimental setup used to detect nanobubble signals using a hydrophone.

FIGS. 24B-24C illustrate example malaria-positive nanobubble signals detected in a nanobubble signal-only model.

FIG. 25A illustrates an example signal for a vapor nanobubble generated in human skin, with significant damping of the second spike.

FIG. 25B illustrates an example signal for a vapor nanobubble generated in skin with the second spike not severely dampened by the skin.

FIG. 25C illustrates dependence of the positive amplitude of the first spike of the signal on the nanobubble lifetime in the gold-water model in water and in skin at three different levels of the energy of the excitation laser pulse, with the skin dampening effect on the nanobubble lifetime (interval) and the signal amplitude seen for skin vs water.

FIGS. 25D-25E illustrate histograms of a nanobubble lifetime for water and skin under identical conditions.

FIG. 25F illustrates an example signal for a vapor nanobubble generated in skin with the second spike fully dampened after the initial expansion (first spike) of vapor nanobubble no collapse (no second spike) occurs in skin.

FIG. 25G illustrates dependence of the second spike to first strike amplitude ratio on the lifetime of a nanobubble generated in the gold-water and gold-water-skin models. Compared to water, skin effect results in a broader ratio of the amplitudes of second to first spikes, which is the effect of dampening of the skin on the expanding and collapsing nanobubble.

FIG. 26A illustrates schematically a skin sample and top-mounted optical excitation and acoustic detection.

FIGS. 26B and 26C illustrate positions of the optical fiber and hydrophone (tilted) in experiments with the skin.

FIG. 26D is an example microscopy image of a skin surface of a dark skin.

FIG. 26E illustrates a full signal of an intact dark skin, so called background signal caused mainly by non-specific optical absorption of the excitation laser pulse by skin melanin and associated photo-acoustic effect.

FIG. 26F illustrates the first spike of the full signal of FIG. 26E.

FIGS. 26G and 26H illustrate signals of intact dark skin on which the first and second laser pulses were applied to the same location: the amplitude of the first spike (caused by the photo-acoustic effect in melanin) does not significantly change, there is a little to none decay of the signal amplitude.

FIGS. 261 and 26J illustrate signal of a dark parasite-treated skin: the second spike disappears during the second laser pulse, and the amplitude of the first spike decreases during the second laser pulse, there is a decay of the signal amplitude.

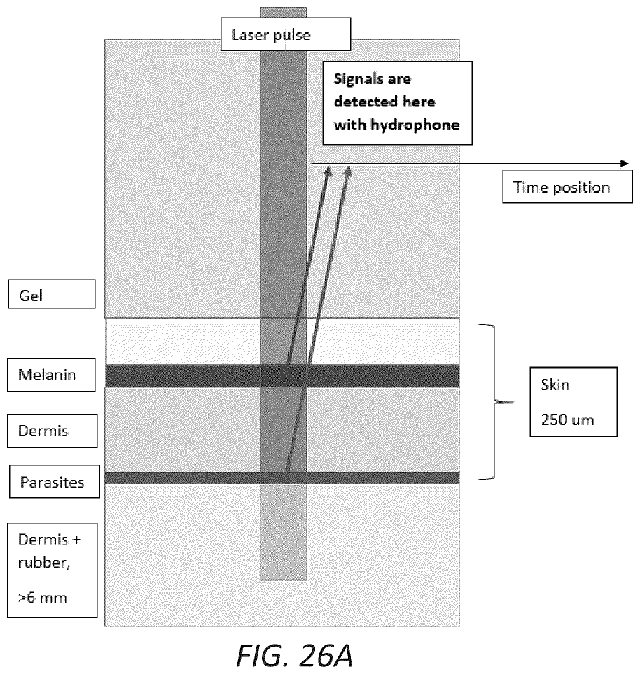

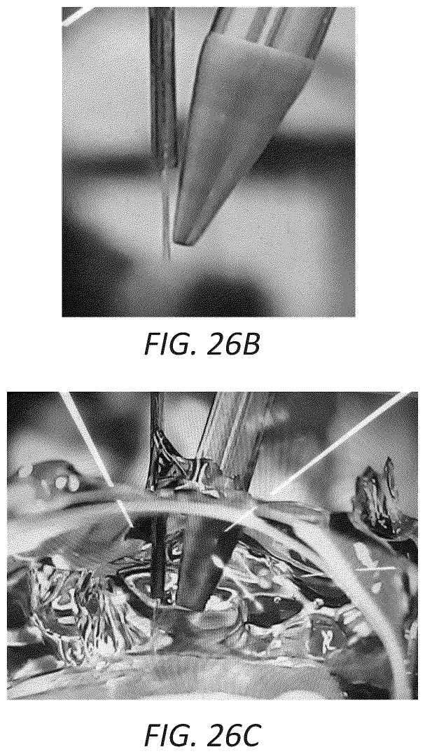

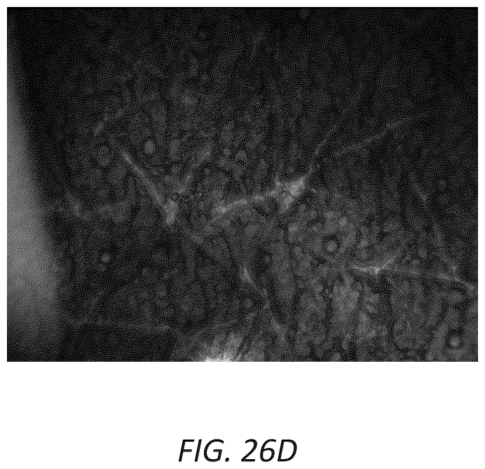

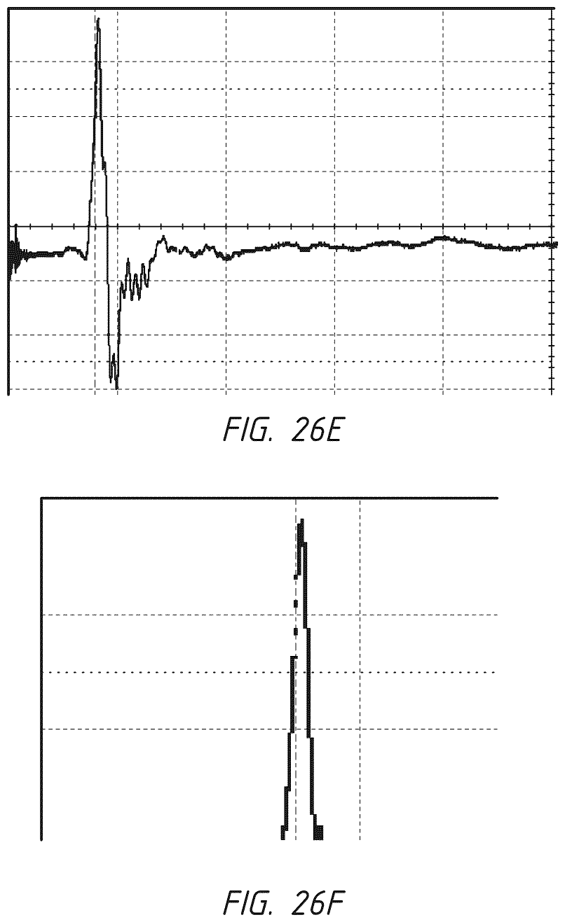

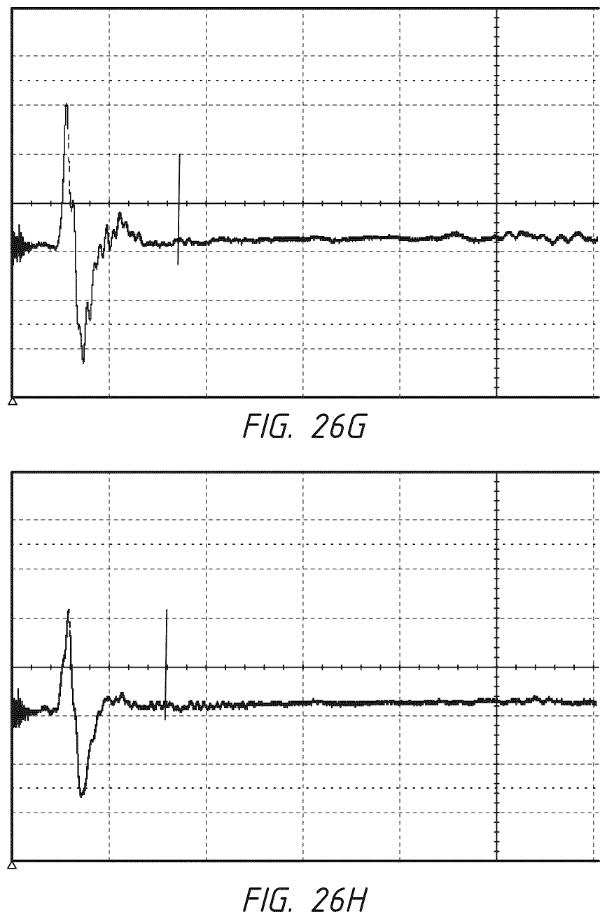

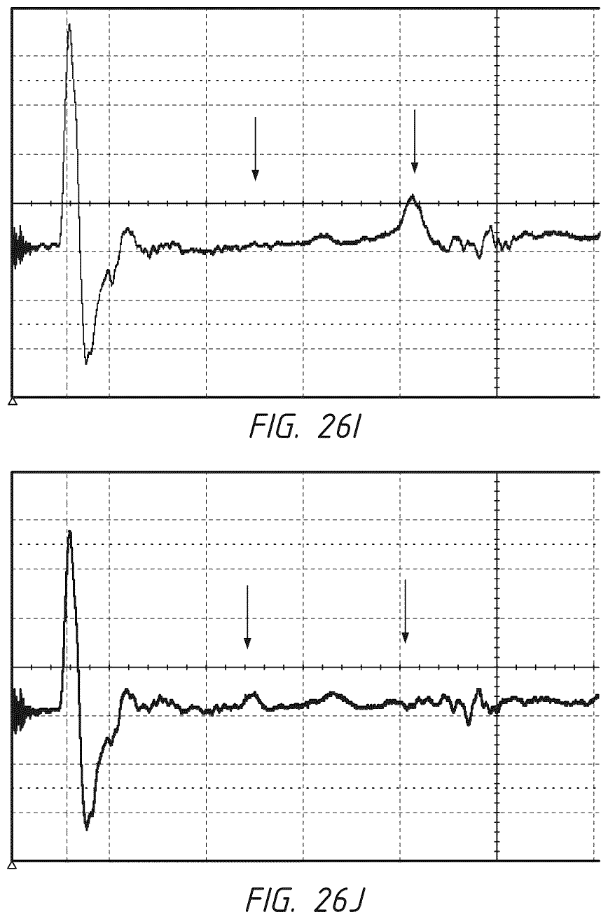

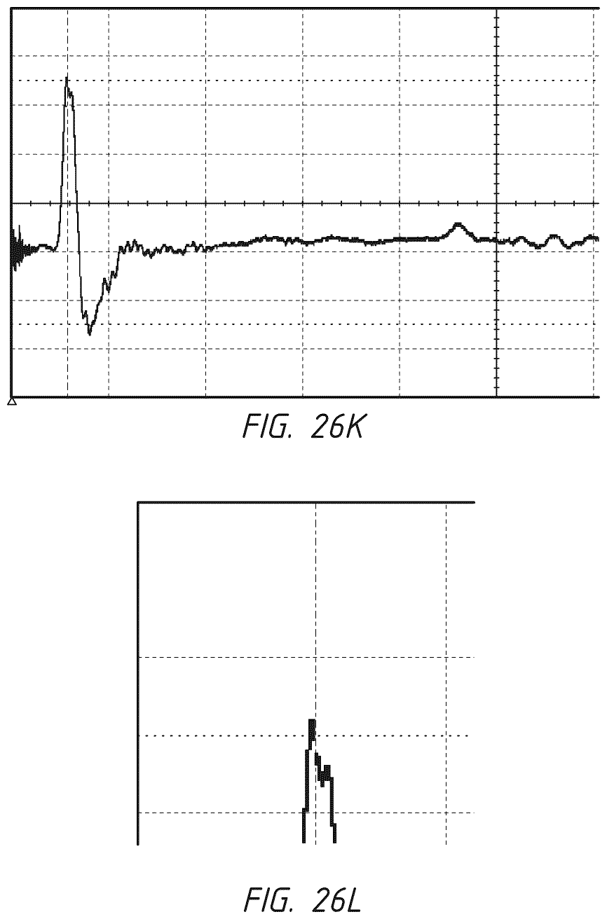

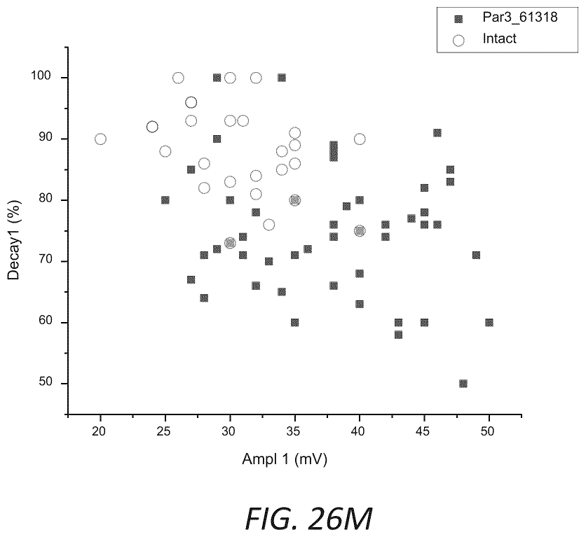

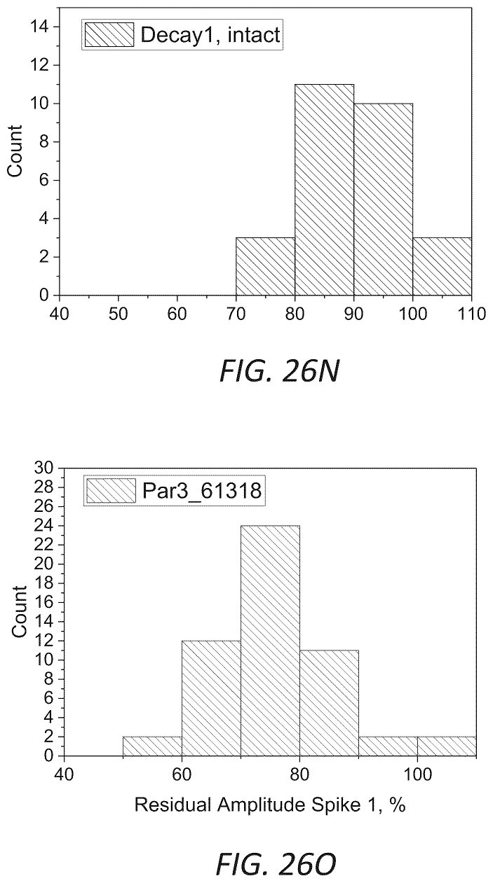

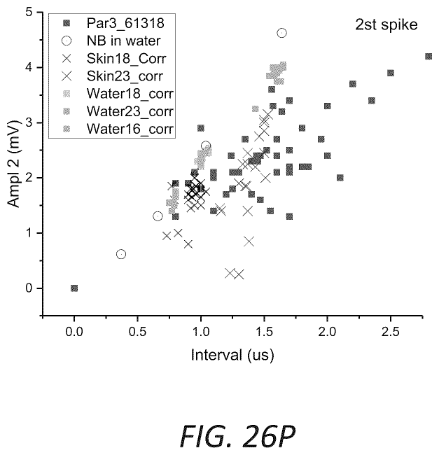

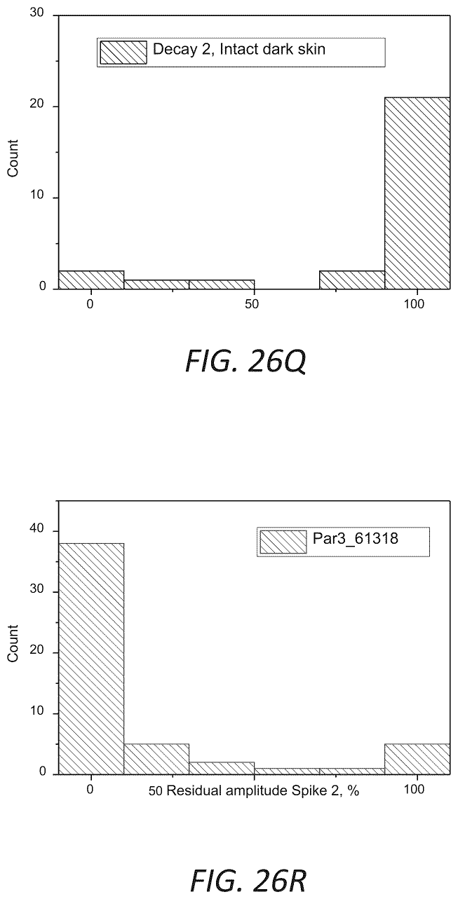

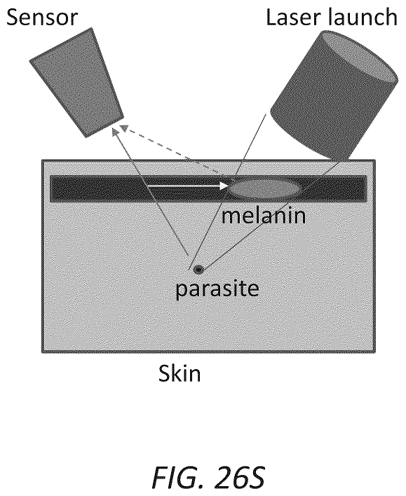

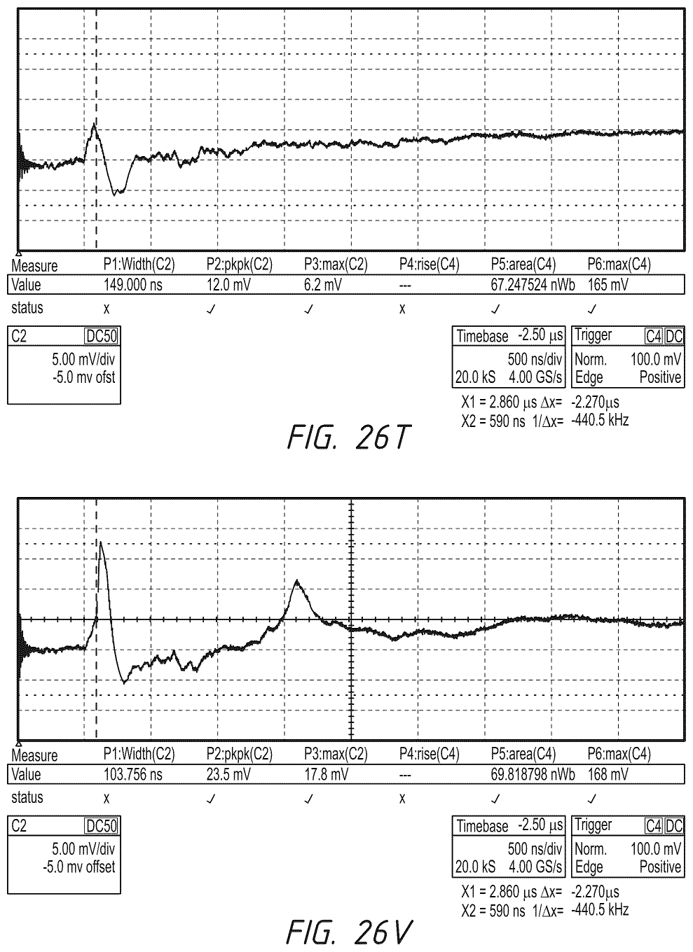

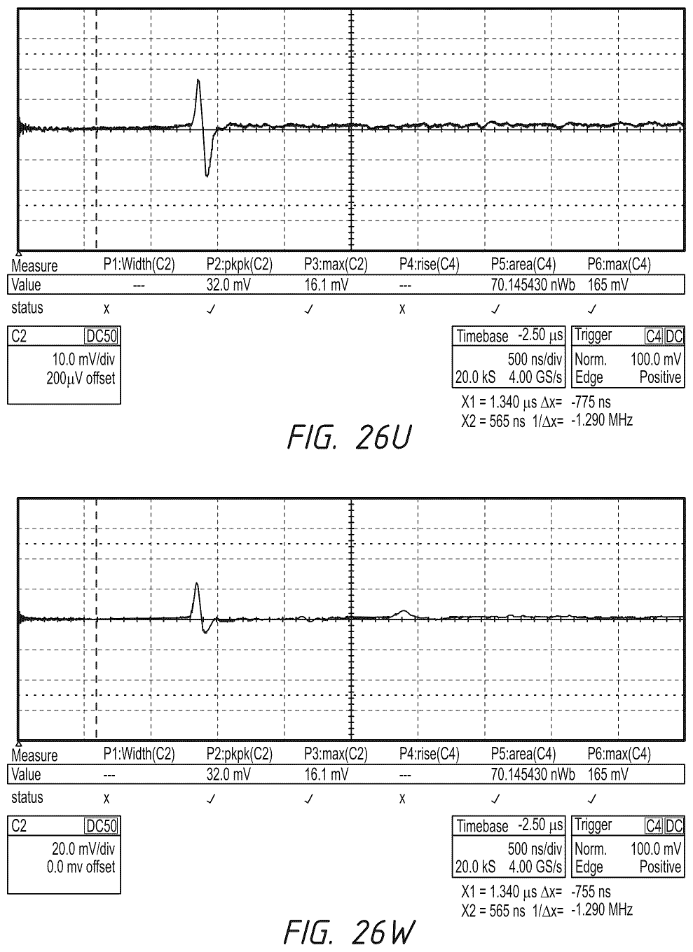

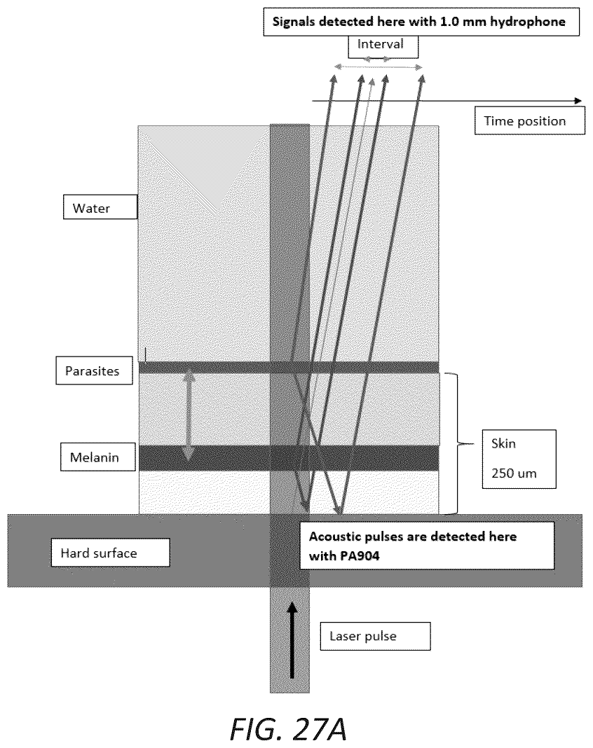

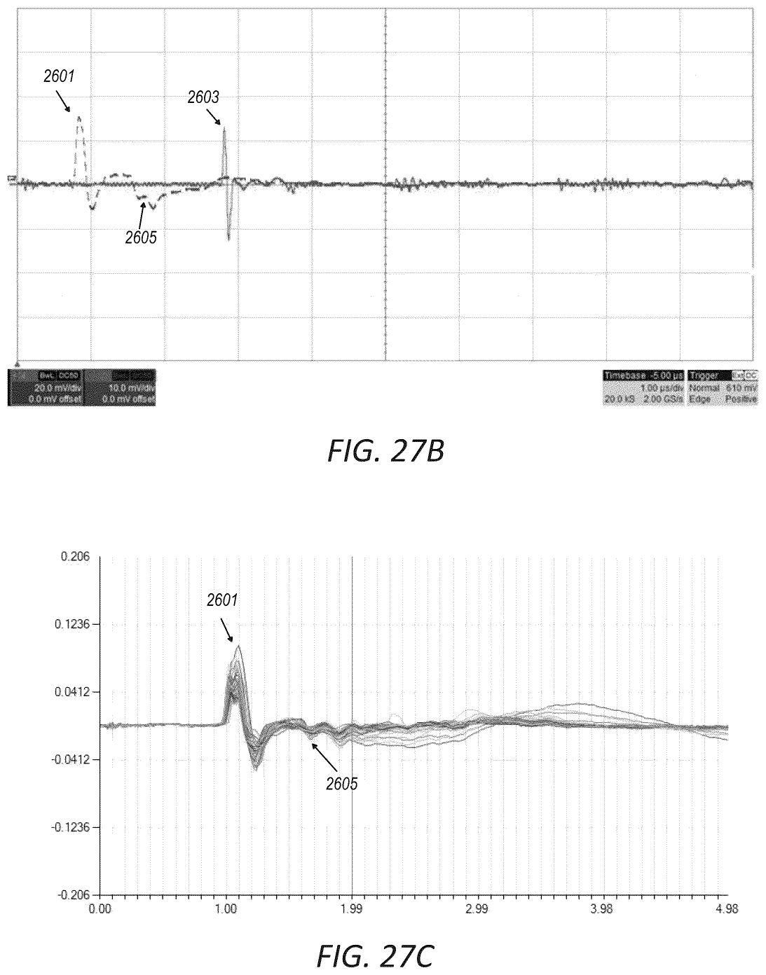

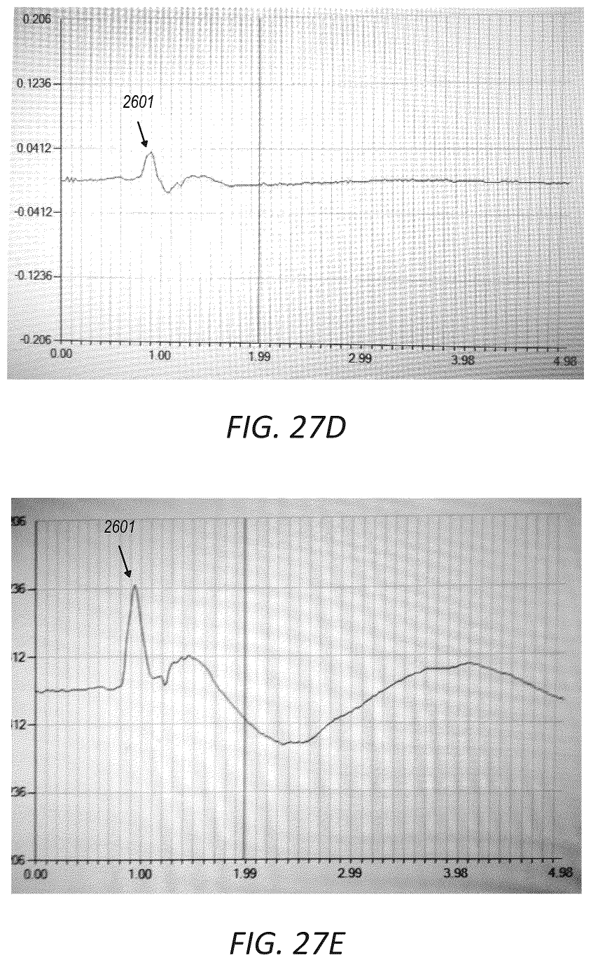

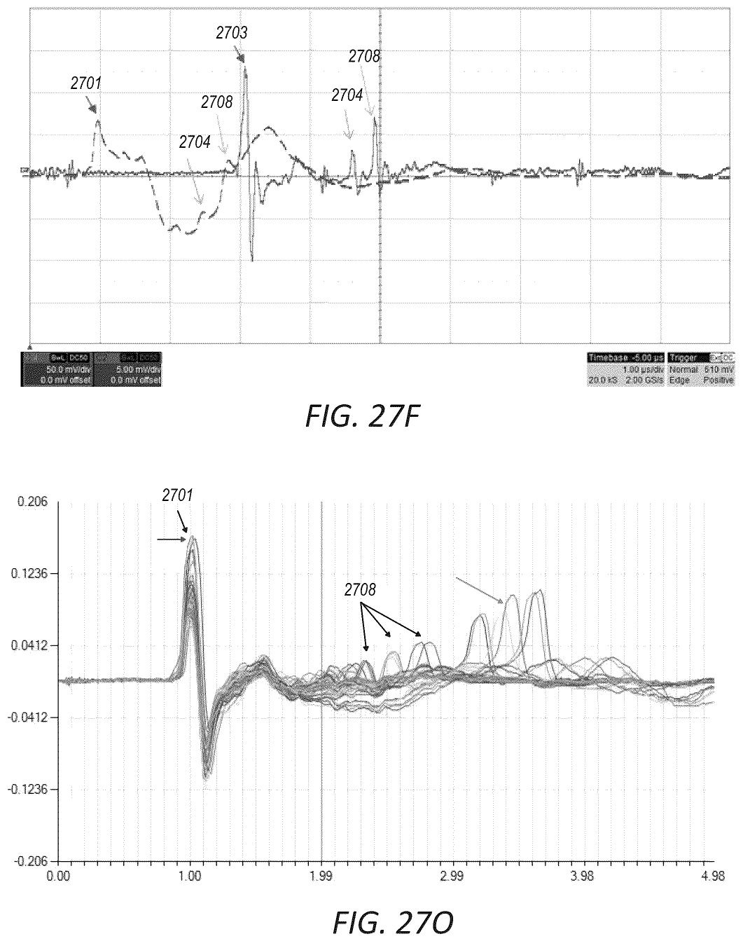

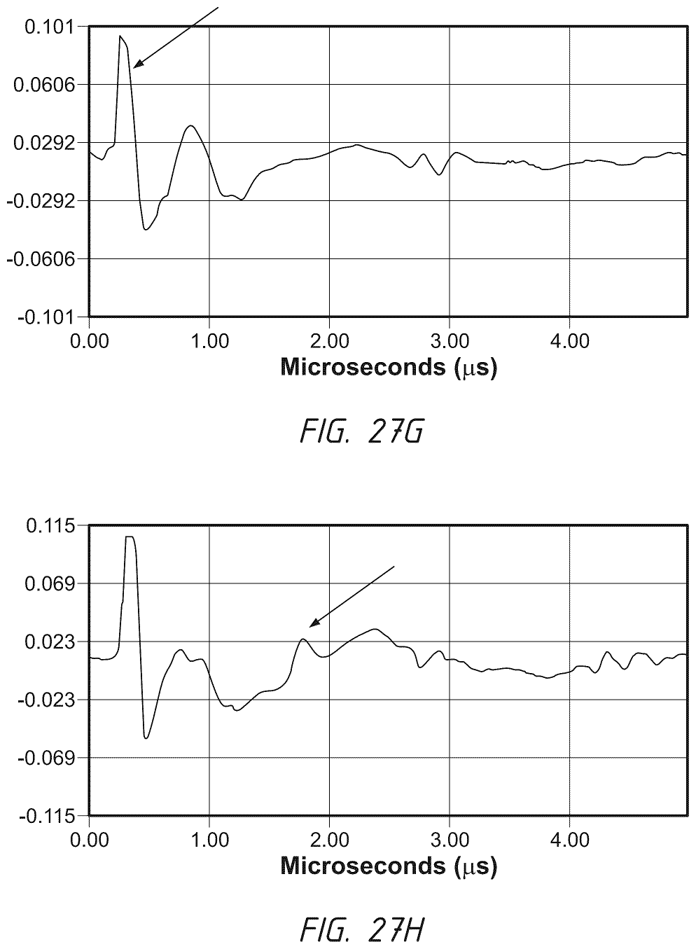

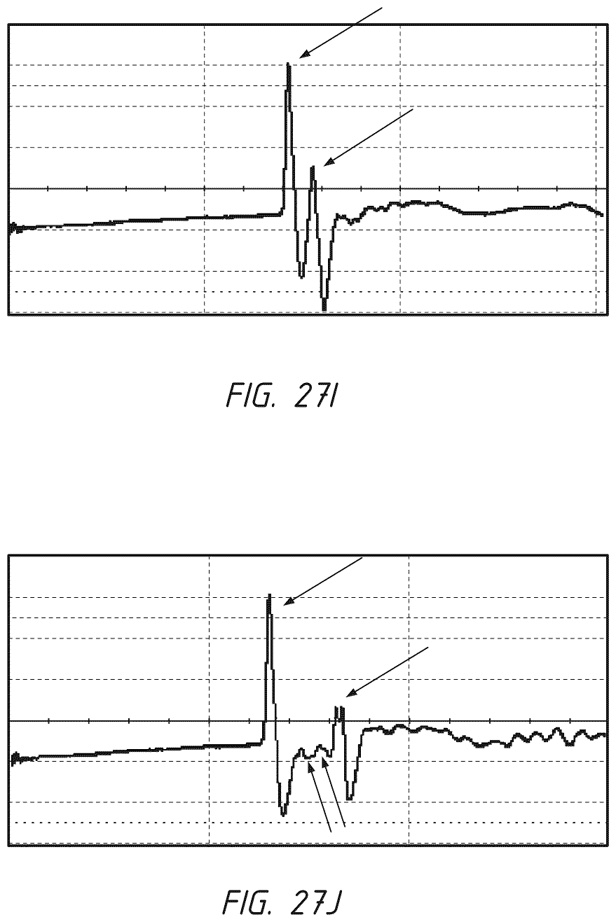

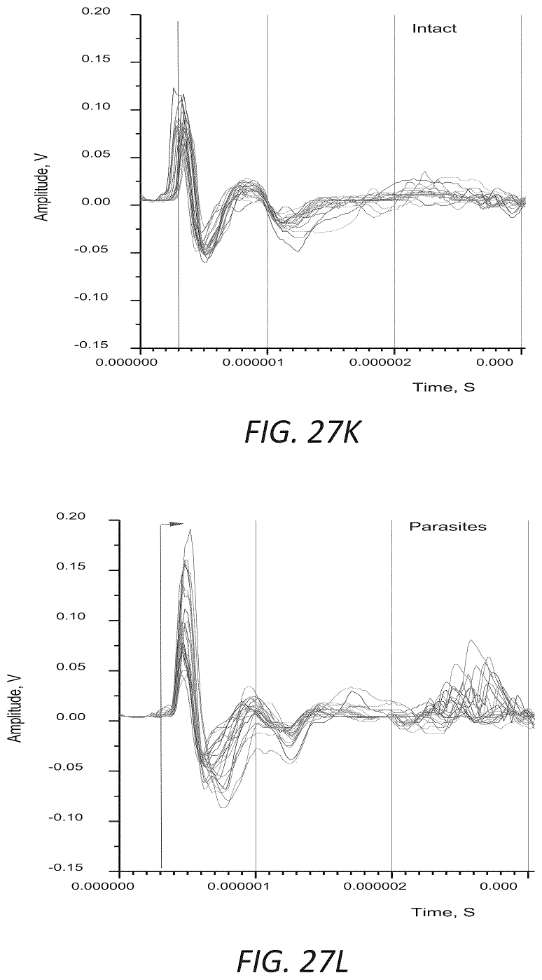

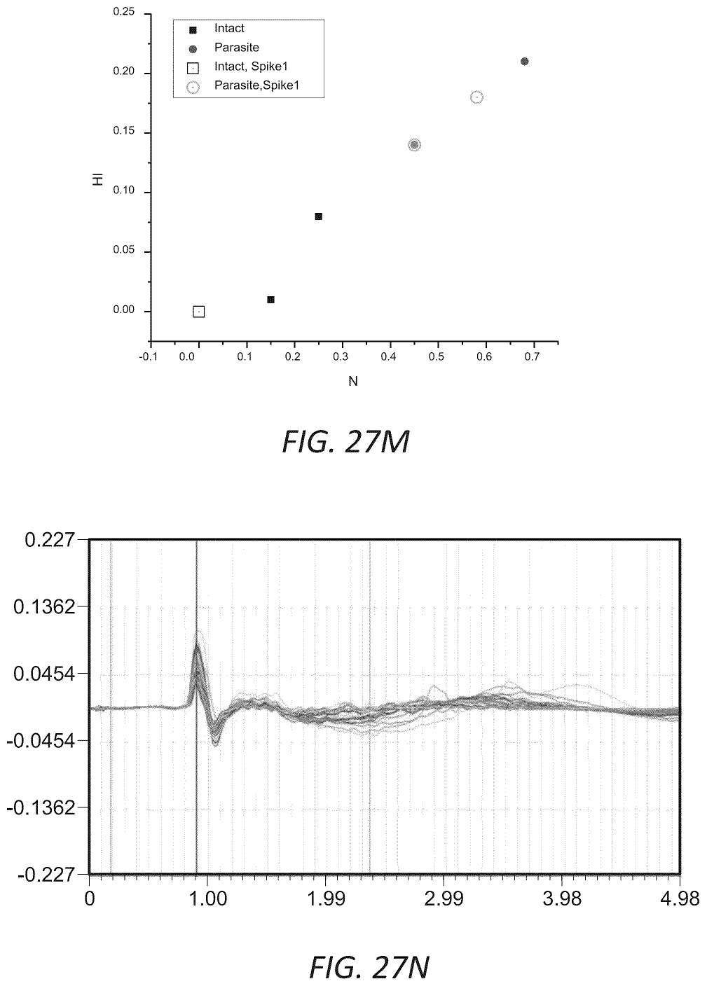

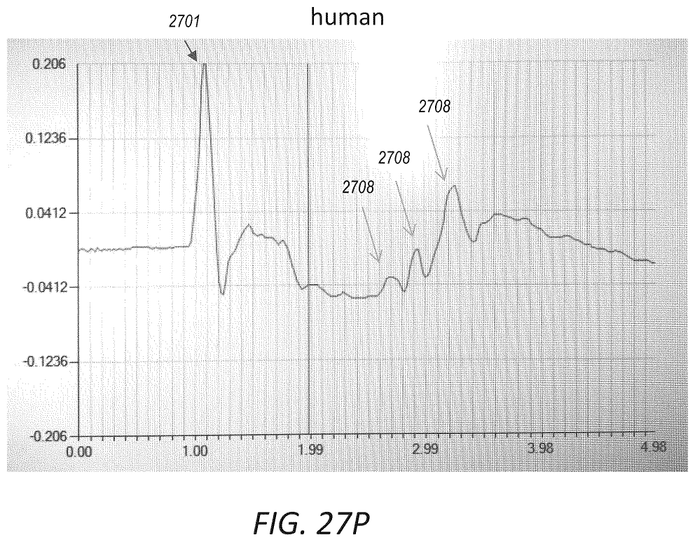

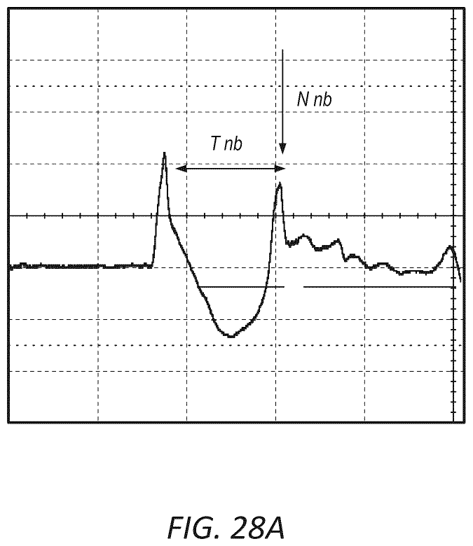

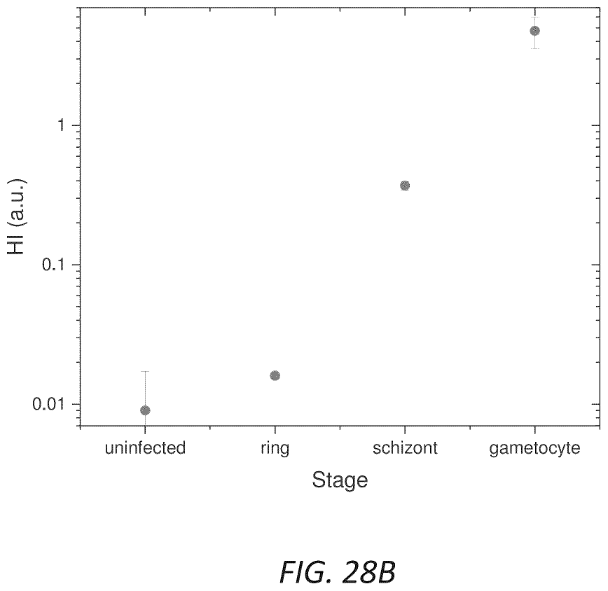

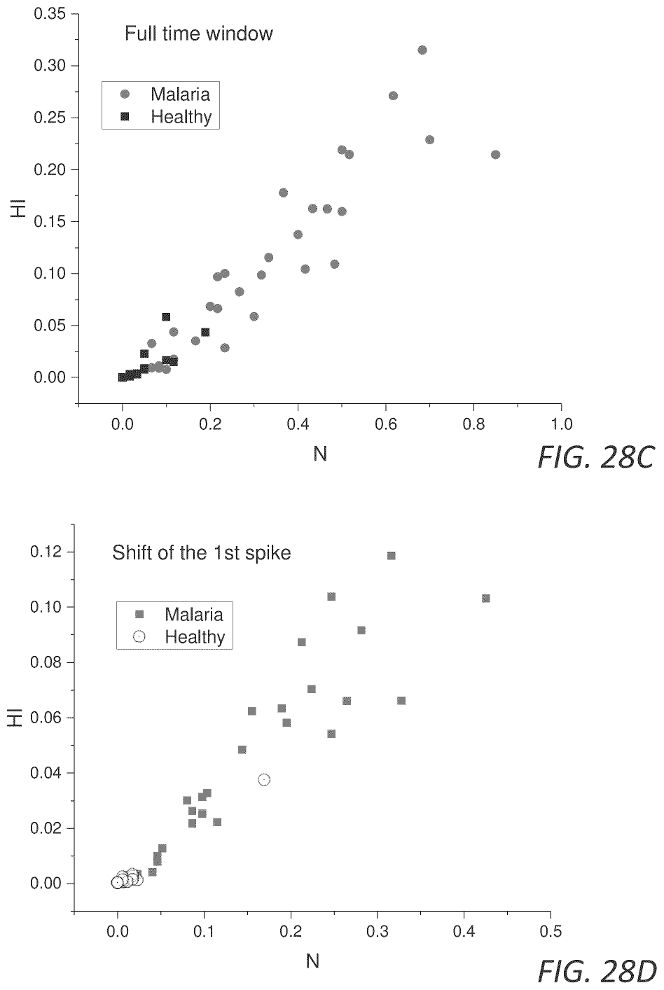

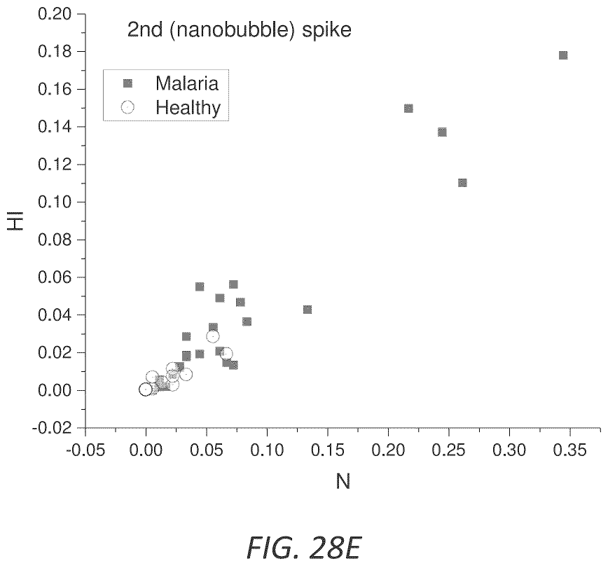

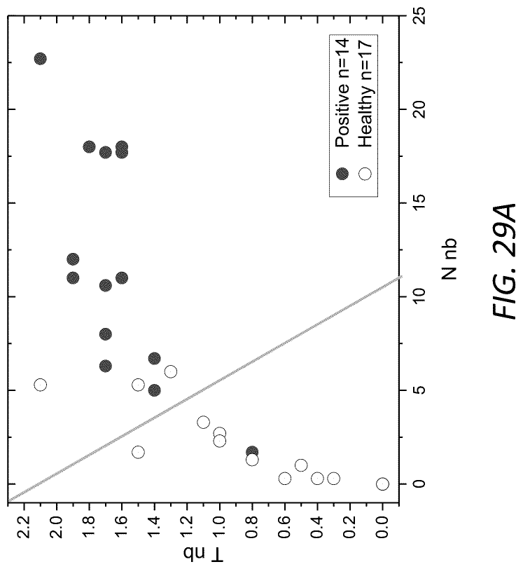

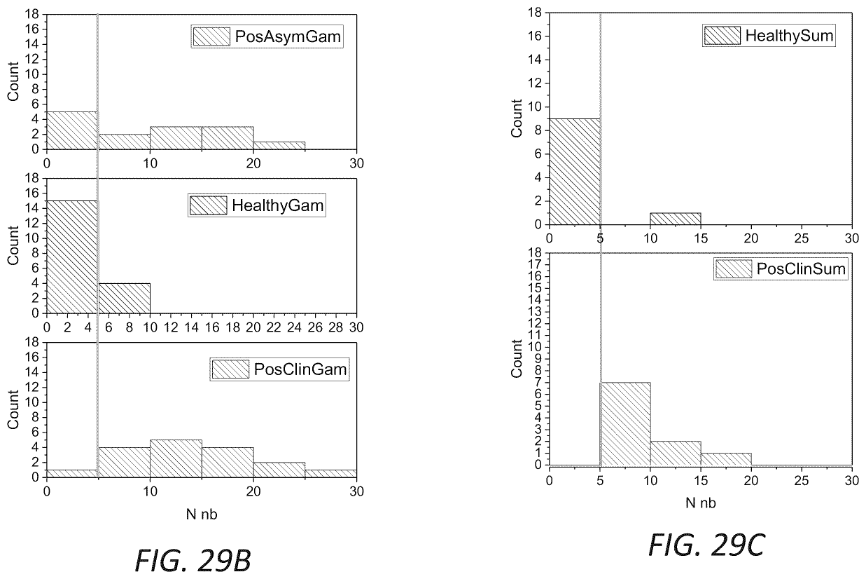

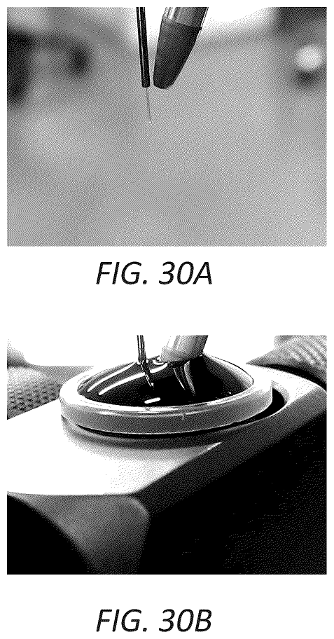

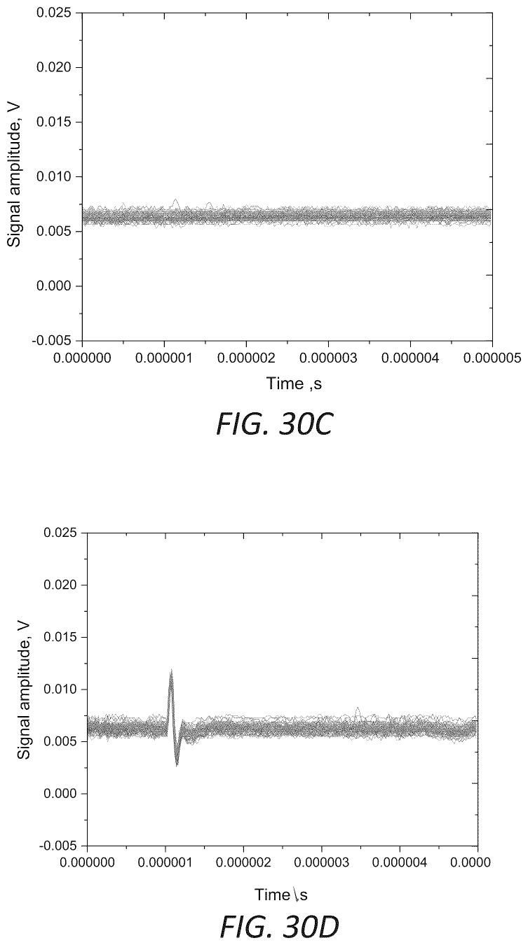

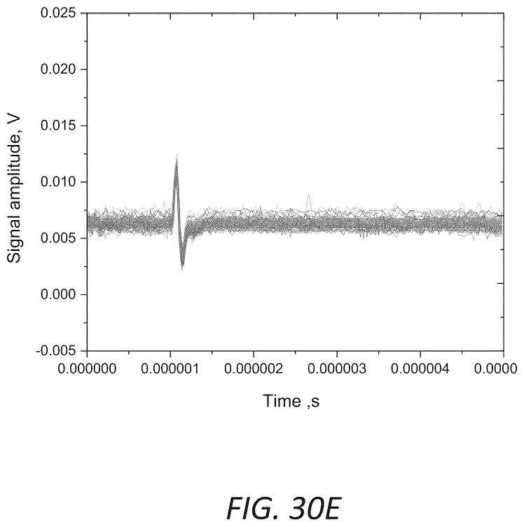

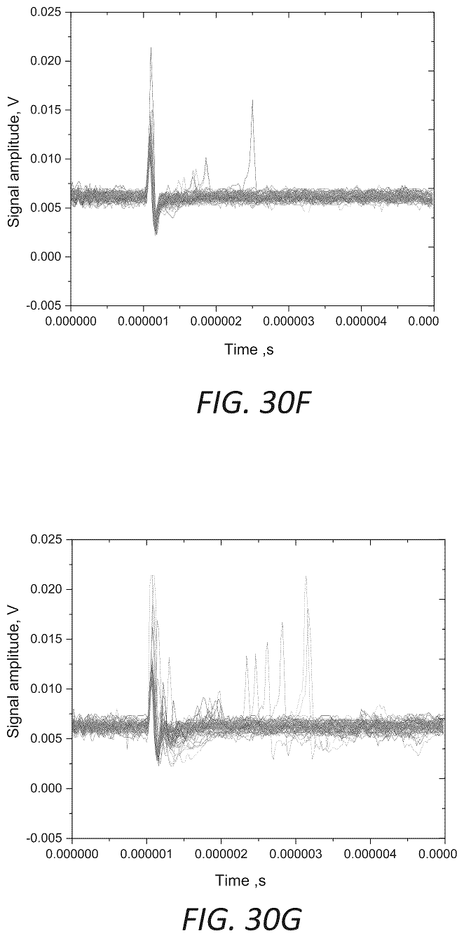

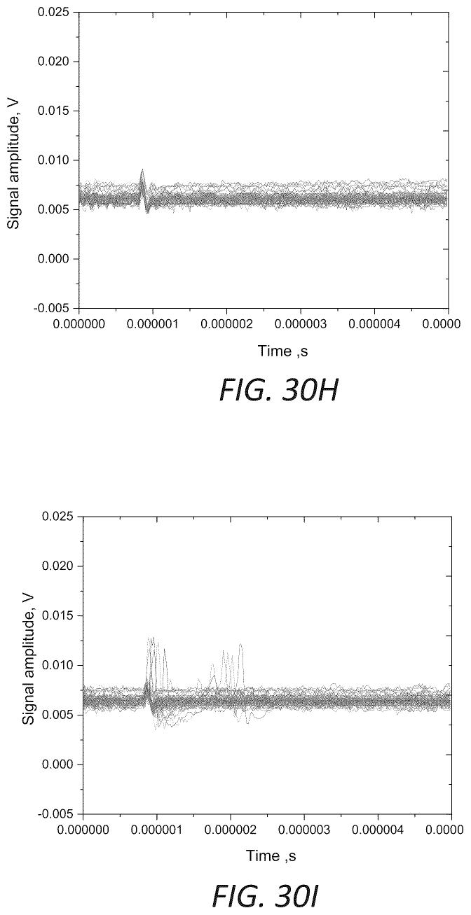

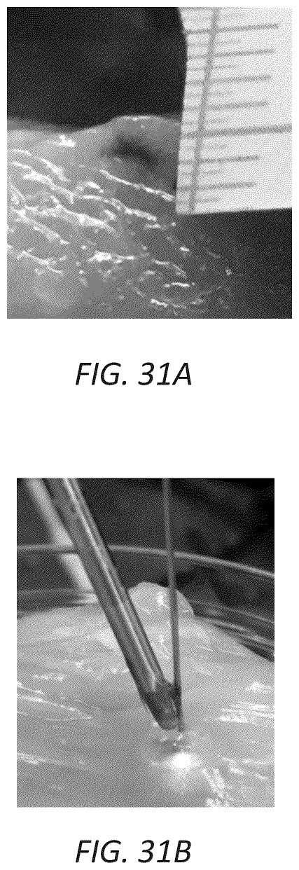

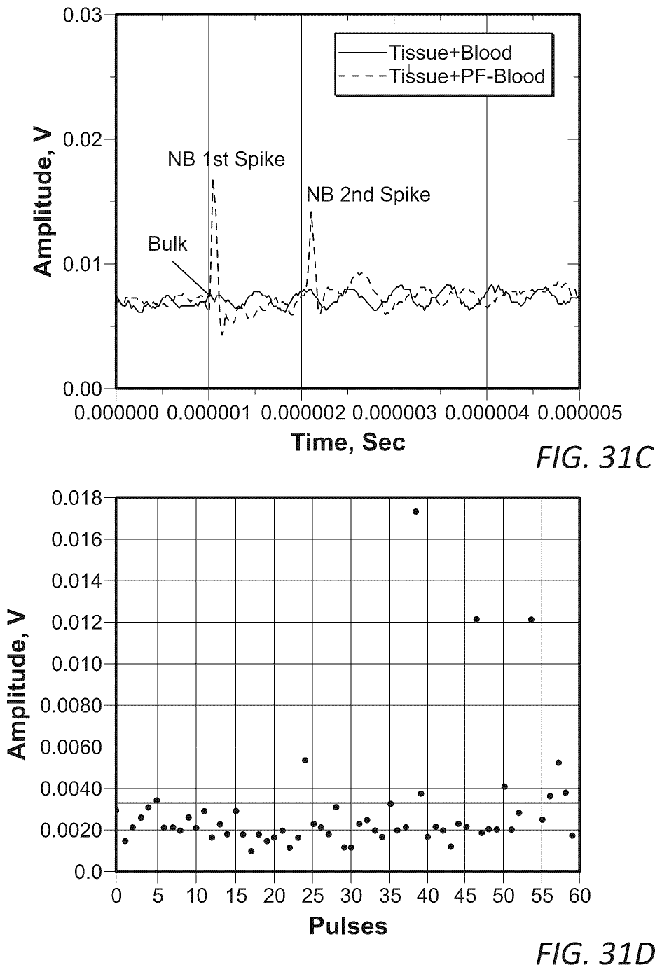

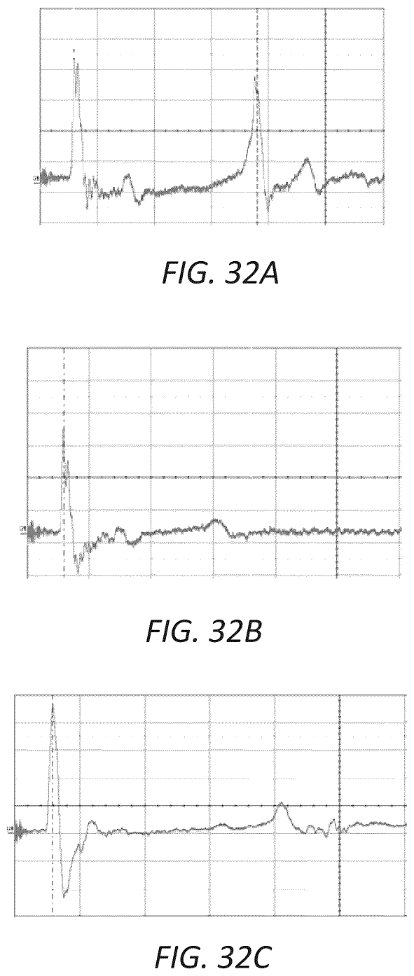

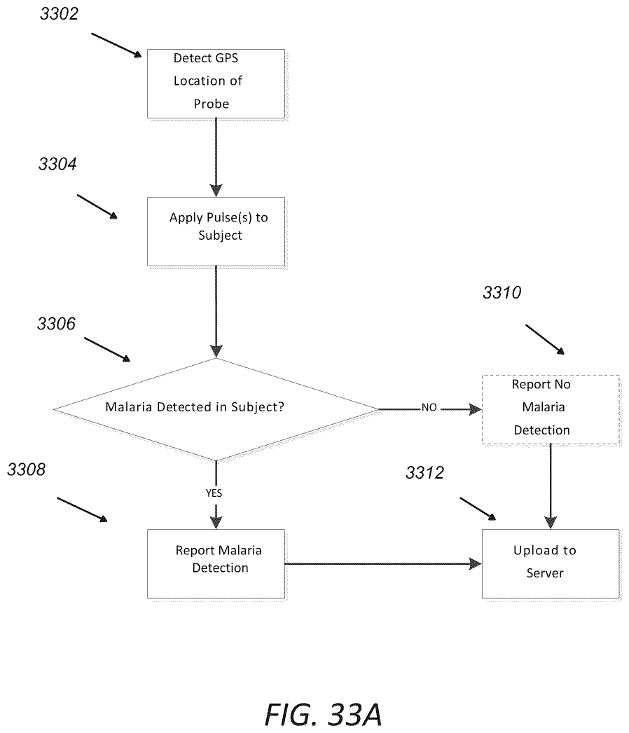

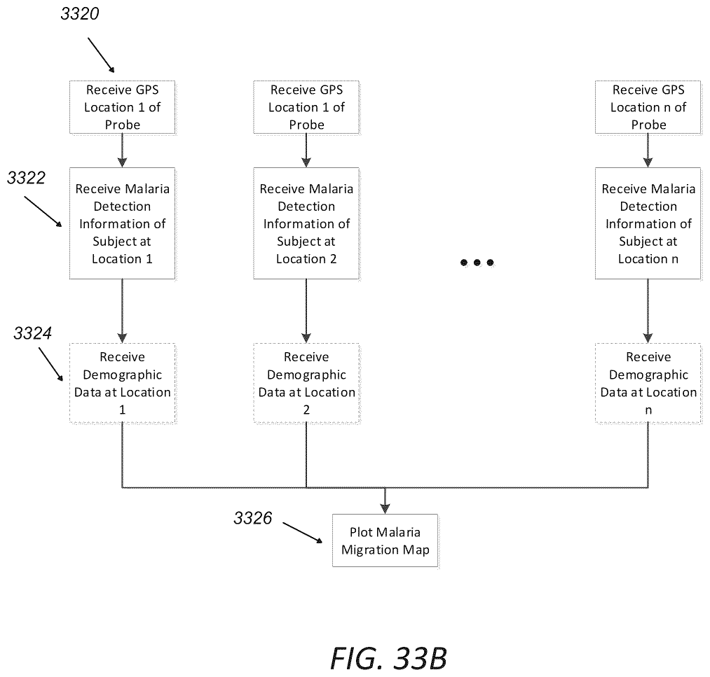

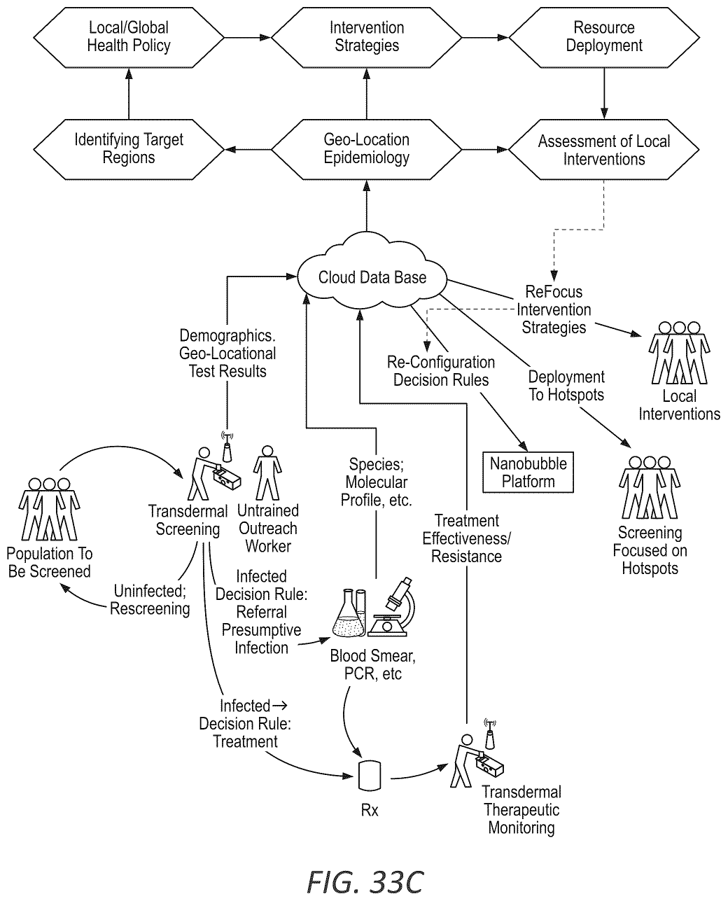

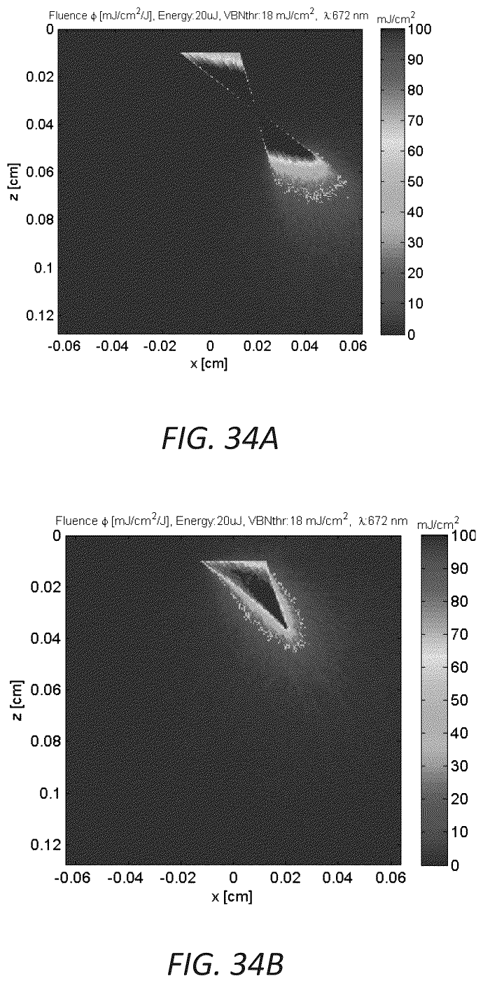

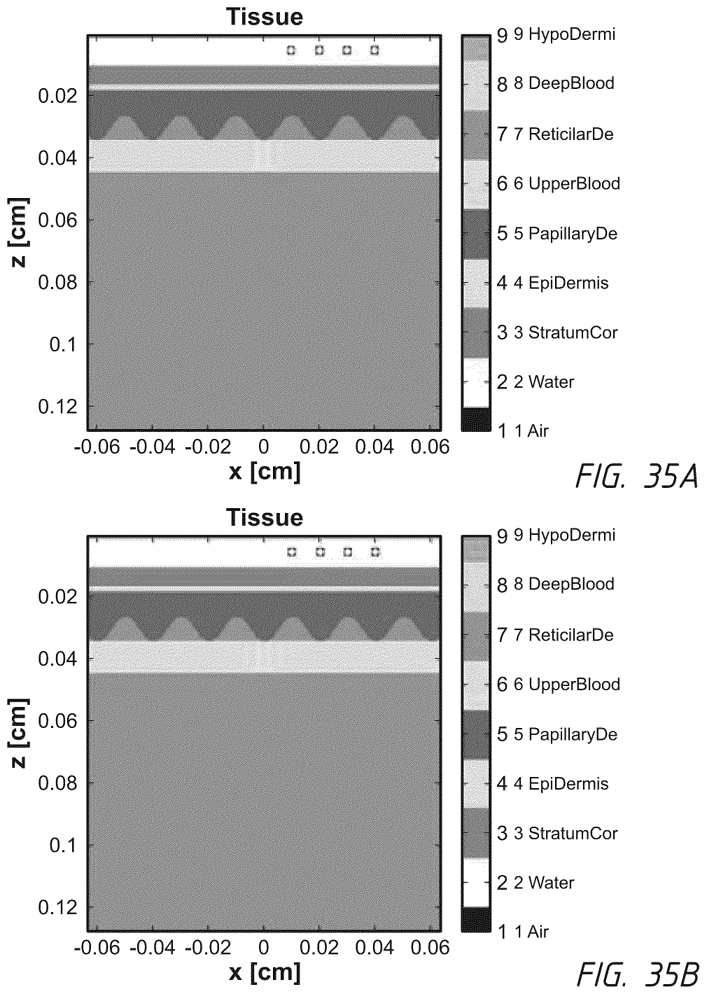

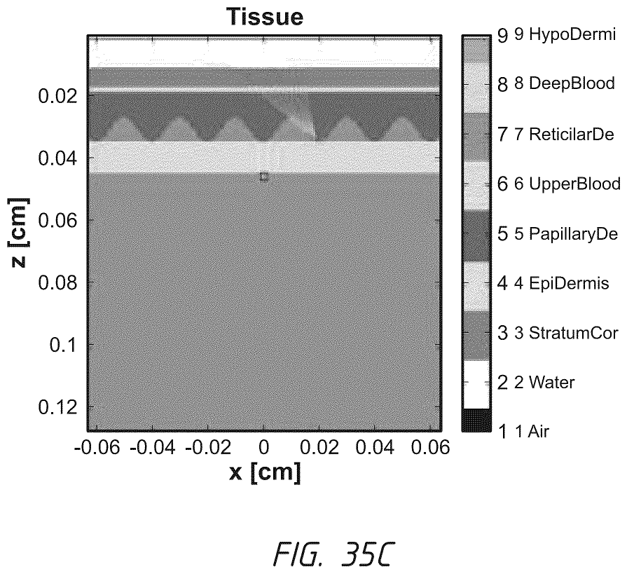

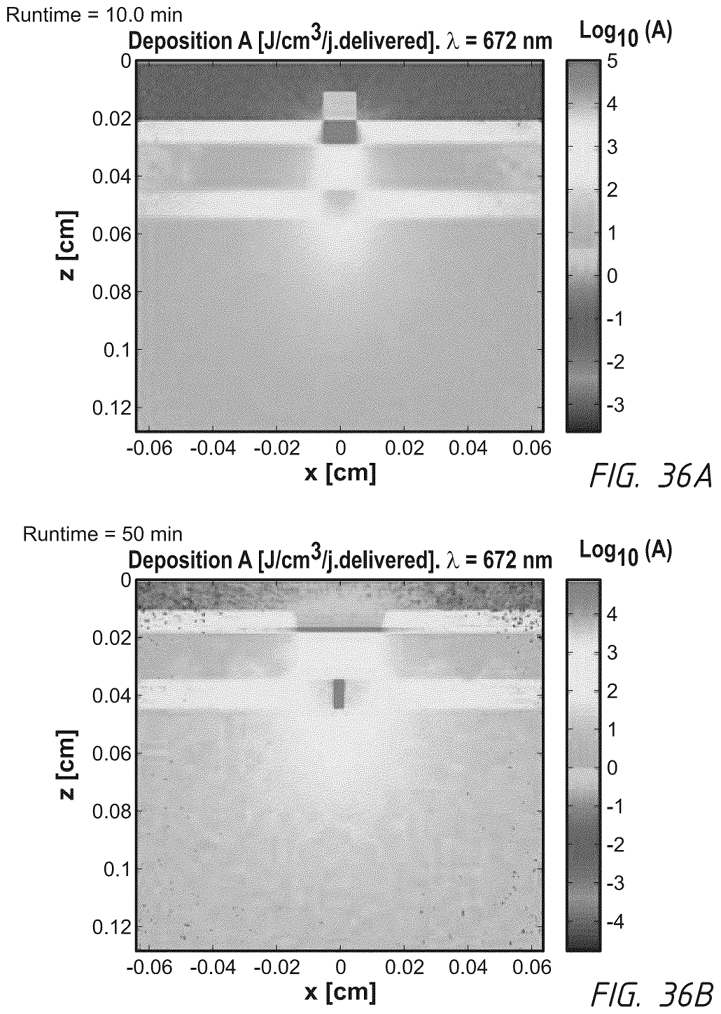

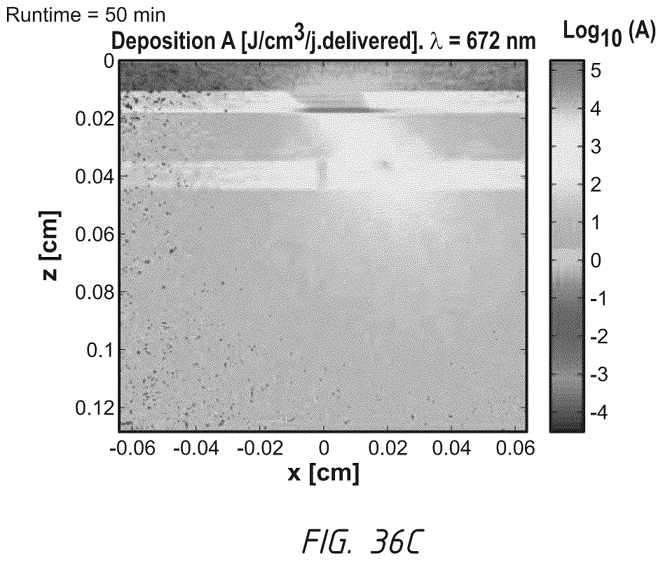

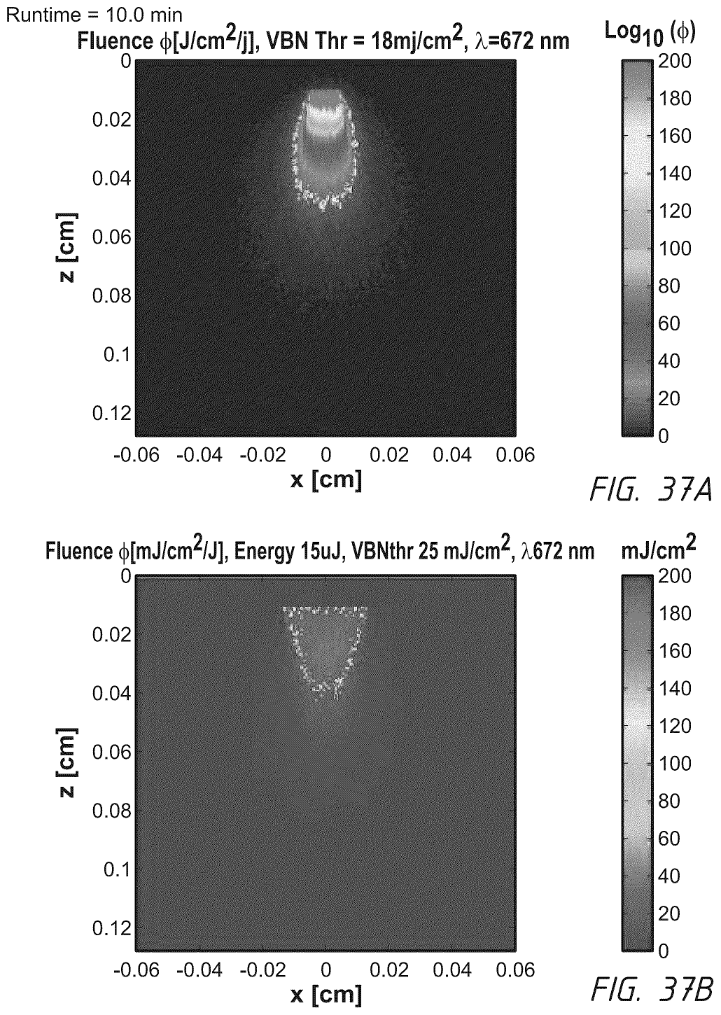

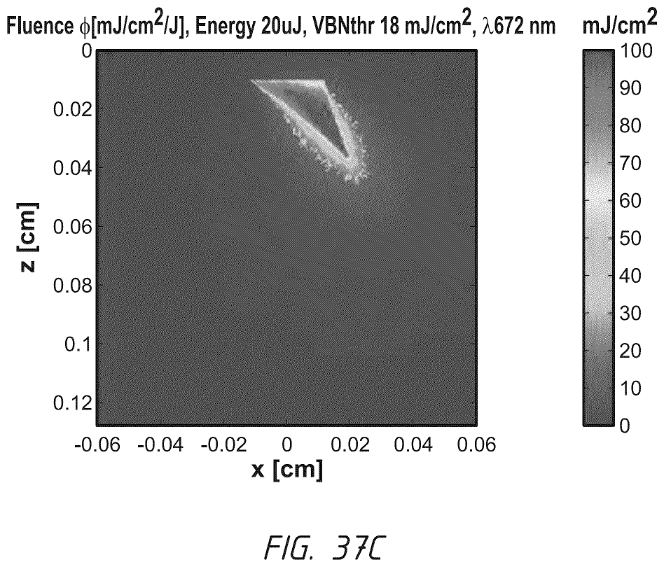

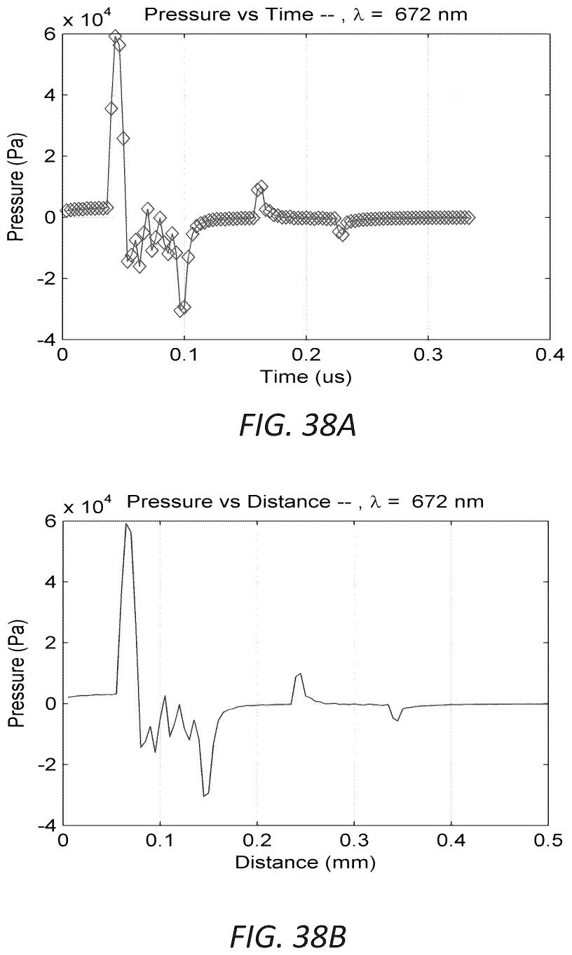

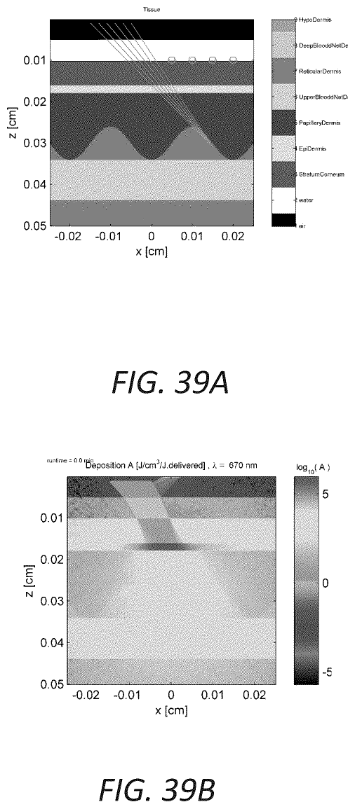

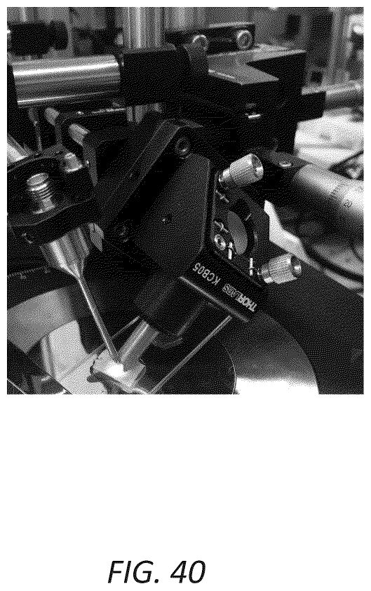

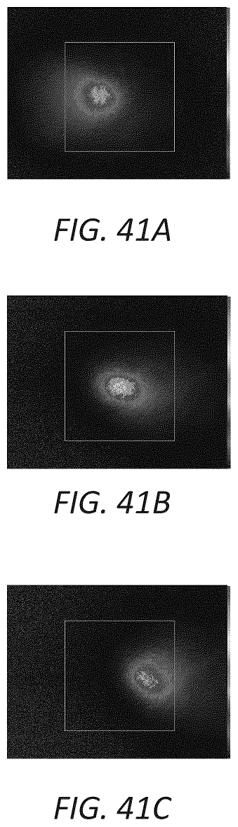

FIG. 26K illustrates a signal of a dark parasite-treated skin obtained after the first laser pulse

FIG. 26L illustrates the doubled first spike of the signal of FIG. 26K.

FIGS. 26M-260 illustrate a decay of the amplitude of the first spike of the signal for intact and parasite-treated dark skin (thickness 250 um) for the two consecutive laser pulses applied to the same skin location.

FIG. 26P illustrates an amplitude of the second (collapse-generated) signal spike as a function of the lifetime (interval between first and second spikes) for the vapor nanobubbles in water (at three pump laser energy levels), and in skin (three identical pump laser energy levels), and in parasite-treated skin.

FIGS. 26Q and 26R illustrate histograms of the residual amplitude (the decay effect) of the second spikes in intact and parasite-treated skin samples. Only parasite-treated sample exhibit a strong decay of the amplitude of the second spike of the acoustic signal

FIG. 26S illustrates schematically a side focused launch of the laser pulse into the skin.

FIGS. 26T-26W illustrate comparison of signals from a direct optical fiber launch of the excitation laser pulse into intact dark skin (26U) and parasite-infected dark skin (26W), with the signals from a side focused launch of the excitation laser pulse into the intact dark skin (26T) and the parasite-infected dark skin (26V). The side launch shows an increase in signal amplitude of parasite signal and the decrease of the amplitude of the background signal obtained from an intact skin.

FIG. 27A illustrates schematically an experimental setup for detection of malaria parasites in skin with a slow-speed detector.

FIG. 27B illustrates example malaria-negative signals detected in a bulk only model in dark intact human skin.

FIGS. 27C-27E illustrate example malaria-negative signals in healthy humans.

FIG. 27F illustrates example signals detected with two sensors in a nanobubble model.

FIGS. 27G and 27H illustrate signals for intact (27G) and parasite-treated (27H) skin samples using a malaria sensor.

FIGS. 271 and 27J illustrate co-registered signals from the reference hydrophone.

FIGS. 27K and 27L illustrate 60 overlaid signals for intact (27K) and parasite-treated (27L) skin samples using a malaria sensor.

FIG. 27M illustrates an N-HI diagram for the malaria probe signals analyzed in the full time window (solid symbols) and in the time window optimized for the analysis of the first spike (hollow symbols) for intact and parasite-treated dark human skin.

FIG. 27N illustrates the malaria probe signals obtained from healthy subjects.

FIGS. 27O-27P illustrate example signals detected in human subjects s infected by the Plasmodium Falciparum malaria.

FIG. 28A illustrates malaria detection metrics based on a second spike in the signal.

FIG. 28B illustrates the signals in blood samples of cultured P. Falciparum parasites as the function of the parasite development stage, from early (ring) to the mature (gametocytes), with the highest signal amplitude in gametocyte stage of malaria parasites

FIGS. 28C-28E illustrate HI-N amplitude of non-invasive signal metrics for an ankle in healthy (n=25) and malaria-positive (n=30) human subjects: full time window includes first and second spikes (28C), first spike only (28D), time window 2.0-5.0 us for the second spike (28E).

FIG. 29A illustrates a Tnb-Nnb diagram for the Plasmodium Falciparum malaria in the Stage 1 study in The Gambia for non-invasive skin signals obtained from healthy and malaria-infected humans.

FIG. 29B illustrates preliminary N nb histograms for non-invasive skin signals obtained from healthy and malaria-infected humans for Plasmodium Falciparum study (The Gambia) for clinical and asymptomatic cases.

FIG. 29C illustrates preliminary N nb histograms for non-invasive skin signals obtained from healthy and malaria-infected humans for the Plasmodium Vivax study (Sumatra) for clinical cases.

FIGS. 30A and 30B illustrate an example experimental setup for optical excitation and acoustic detection of parasites in a liquid sample of whole blood.

FIG. 30C illustrates 60 signals for static water using the setup of FIGS. 30A and 30B.

FIG. 30D illustrates 60 signals for static whole human blood using the setup of FIGS. 30A and 30B.

FIG. 30E illustrates 60 signals for a flow of whole human blood using the setup of FIGS. 30A and 30B.

FIG. 30F illustrates an overlay of 60 hydrophone signals for static blood with 50 p/uL of malaria parasites.

FIG. 30G illustrates an overlay of 60 hydrophone signals for flowing blood with 50 p/uL of malaria parasites.

FIG. 30H illustrates an overlay of 60 signals for uninfected blood under identical laser pulse fluence as FIGS. 30F and 30G but with a smaller optical fiber.

FIG. 30I illustrates an overlay of 60 signals for blood with 50 p/uL of malaria parasites under identical laser pulse fluence as FIGS. 30F and 30G but with a smaller optical fiber.

FIGS. 31A and 31B illustrate an example experimental setup for optical excitation and acoustic detection of parasites in a chicken breast model.

FIGS. 31C and 31D illustrate, respectively, signals and signal amplitude logs for the experimental setup of FIGS. 31A and 31B for intact (31C) and parasite-treated (31D) tissues.

FIGS. 32A-32C illustrate additional ultrasonic signals from a single transient vapor nanobubble in -water model (32A), in skin model (32B), and around malaria parasites in human skin (32C).

FIGS. 33A and 33B illustrate example processes of tracking migration of malaria using GPS.

FIG. 33C illustrates geo-tagging of malaria transmission.

FIG. 34A illustrates propagation of a side-launched laser beam in water.

FIG. 34B illustrates propagation of the side-launched focused laser beam in dark human skin.

FIG. 35A illustrates simulation of skin model with a standard optical fiber of 100 um core.

FIG. 35B illustrates simulation of skin model with a focused beam with an incident diameter of 300 um.

FIG. 35C illustrates simulation of skin model with a side-launched focused beam with an incident diameter of 250 um.

FIG. 36A illustrates simulation of deposition of optical energy with a standard optical fiber of 100 um core.

FIG. 36B illustrates simulation of deposition of optical energy with a focused beam with an incident diameter of 300 um.

FIG. 36C illustrates simulation of deposition of optical energy with a side-launched focused beam with an incident diameter of 250 um.

FIG. 37A illustrates simulation of the fluence delivered, the area of the generation of vapor nanobubbles for the threshold of 18 mJ/cm² with a standard optical fiber of 100 um core.

FIG. 37B illustrates simulation of the fluence delivered, the area of the generation of vapor nanobubbles for the threshold of 18 mJ/cm² with a focused beam with an incident diameter of 300 um.

FIG. 37C illustrates simulation of the fluence delivered, the area of the generation of vapor nanobubbles for the threshold of 18 mJ/cm² with a side-launched focused beam with an incident diameter of 250 um.

FIG. 38A illustrates a signal detected by an acoustic sensor as the pressure vs time in the computer model.

FIG. 38B illustrates pressure detected vs distance from the skin surface in the computer model.

FIGS. 39A and 39B illustrate example data relating a side focused launch of the excitation laser pulse.

FIG. 40 illustrates an example experimental prototype of the side-launching system, in which the focusing part is made of three lenses, the launching lens is side-polished the direct the beam into the skin at the angle of 45 degrees.

FIGS. 41A-41C are images of the excitation beam as it was launched into water from the right side show the intensity profile at the different depths. 41A: the post-focal lower zone; 41B: the focal zone at 200 um depth (the parasite level); 41C: laser spot 150 um above the focal zone is laterally shifted by 100 um and has lower intensity at the level of melanin location in skin. White square has the size of 500 um and allows to estimate the lateral shift of the excitation beam.

FIGS. 42A and 42B illustrate optical intensity profile at the tip of the optical fibers.

FIGS. 42C and 42D illustrate optical intensity profile at the bottom of 250 um dark human skin.

### DETAILED DESCRIPTION

Although certain embodiments and examples are described below, this disclosure extends beyond the specifically disclosed embodiments and/or uses and obvious modifications and equivalents thereof. Thus, it is intended that the scope of this disclosure should not be limited by any particular embodiments described below.

The test parameters and experimental data provided in the present disclosure show preliminary results collected from in-field testing in early-stage human studies. They are disclosed herein as the evidence and examples of the first non-invasive detection of malaria in humans. They also provide insight into the principle of operation of laser-induced transient vapor nanobubbles for detecting malaria in human patients in non-invasively and/or minimally invasive manners. The sensor prototypes, experimental hardware and software are in early stages of development and the results obtained are thus process-dependent. The results and the processes used for collecting the results have not been scientifically peer-reviewed. However, the results were obtained under a controlled environment in blinded studies. That is, the personnel responsible for signal collection did not know the malaria status of patients at the time of signal collection. Further, human data were obtained and documented under an Institutional Review Board (TRB)-approved protocol and by using WHO-approved reference methods for determining the malaria status of each subject studied. Accordingly, it may be that the protocols, data acquisition and validation procedures, and scientific results and conclusions discussed herein could be subject to further analysis and future study.

### Overview of Example Systems and Processes of Diagnosing and/or Treating Malaria with Transient Vapor Nanobubbles (“Nanobubbles”)

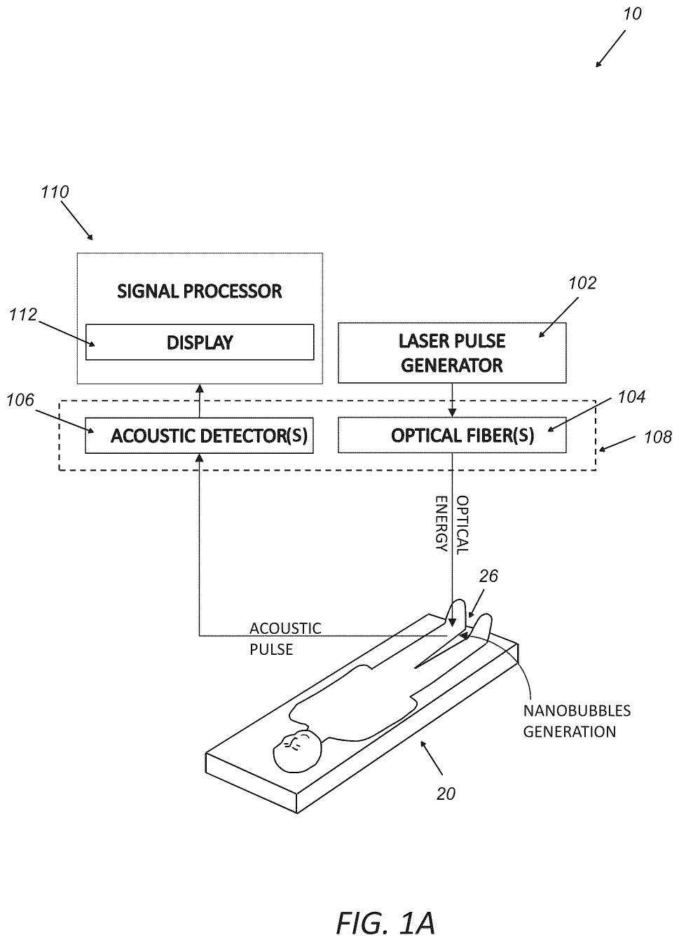

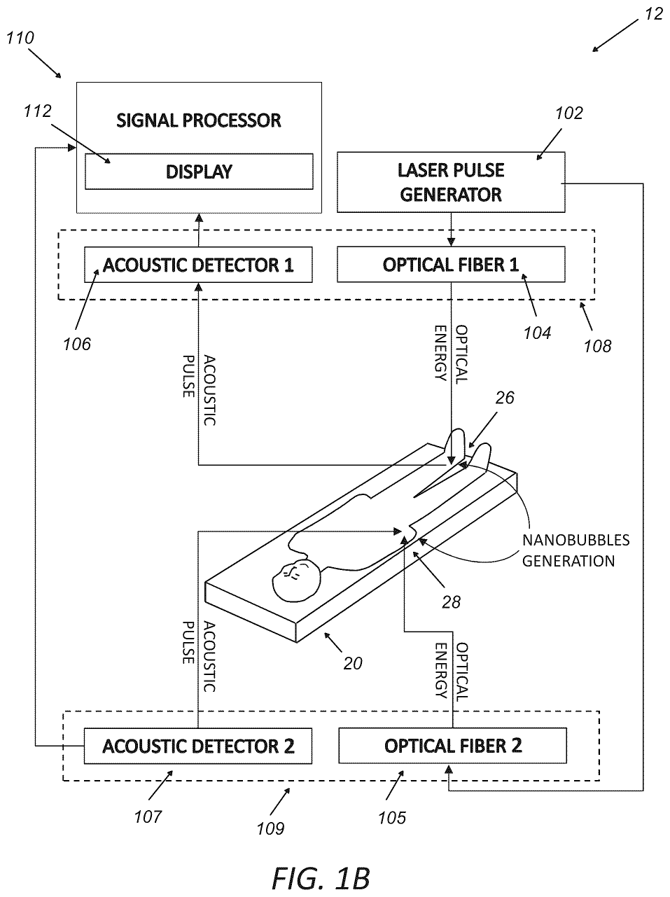

FIG. 1A illustrates schematically a system 10 for noninvasively diagnosing and/or treating malaria. The system 10 can have an optical source for providing laser pulses to a test subject 20. Although a patient is illustrated in FIG. 1A as the test subject 20, a person skilled in the art will appreciate from the disclosure herein that the test subject 20 can include any other test subjects that may have been infected by malaria parasites, including but not limited to a blood and/or tissue sample.

The optical source can include a laser pulse generator 102 and one or more optical fibers 104. The laser pulse generator 102 can generate optical energy, which can be laser pulses of predetermined energy and/or fluence levels. The optical fiber(s) 104 can deliver the generated laser pulses to a location 26 on the test subject 20. Using the patient as an example, the optical fiber(s) 104 can direct laser pulses to any suitable locations on the patient, such as on the digits, hand, wrist, ankle (such as shown in FIG. 1A), neck, earlobes, lips, under the patient's tongue (tongue base), or other locations.

If the test subject 20 has been infected by malaria parasites, the test subject 20 can contain malaria-specific nanoparticles, such as HZ (hemozoin) nanocrystals. HZ (hemozoin) nanocrystals have a significantly higher optical absorbance than that of an uninfected red blood cell, uninfected hemoglobin, or major proteins in the red blood cell. The malaria-specific nanoparticles can likely be present at the location 26 as malaria parasites can travel to various locations in the patient by blood.

If the malaria-specific nanoparticles are located within a depth from a surface of the location 26 that can be penetrated by the laser pulses, laser-induced transient vapor nanobubbles (“nanobubbles”) can be generated at the location 26 underneath the surface of the location 26. Nanobubbles are a transient phenomenon. The generation of nanobubbles can produce sound waves.

The system 10 can have one or more acoustic detectors 106 configured for detecting the sound waves or an acoustic pulse of the nanobubbles generation. Close proximity of the acoustic detector(s) 106 and the acoustic pulse source, which is/are the nanobubble(s), can enhance the sensitivity and specificity of the acoustic detector(s) 106. This can be due to the acoustic pulse being stronger near the source than further away from the source, in particular for a point source. A nanobubble is a point source that generates spherical wavefronts. If the acoustic detector (s) 106 is(are) too far away from the source, the signal reaching a surface of the acoustic detector(s) 106 may be too weak to be detected. A commonly used acoustic detector can be a piezo element. The piezo element can generate an electrical charge in response to vibrations caused by the pressure wave.

In some embodiments of the present disclosure, the optical fiber(s) 104 and the acoustic detector(s) 106 are located in a single sensor probe 108. The single sensor probe configuration can be easier to use than having two separate probes for an optical fiber and an acoustic detector. The single probe configuration can also allow the acoustic detector(s) 106 to be closer to the nanobubble and to the optical fiber(s) 104 (which can reduce and/or minimize the angle of acoustic incidence) than if the optical fiber(s) 104 and the acoustic detector(s) 106 are in separate probes.

The system 10 can have one or more signal processors and/or controller 110 in electrical communication with the laser pulse generator 102 and/or the acoustic detector(s) 106. The one or more signal processors 110 can process the signals from the acoustic detector(s) 106 to determine if the signals are indicative of nanobubble(s) generation and thus for the presence of malaria-specific nanoparticles. In some embodiments, the signals from the acoustic detector(s) 106 can be amplified before being processed by the one or more signal processors 110. The one or more signal processors 110 can cause the processed signals and/or the detection or non-detection of nanobubble(s) generation to be displayed on a display device 112.

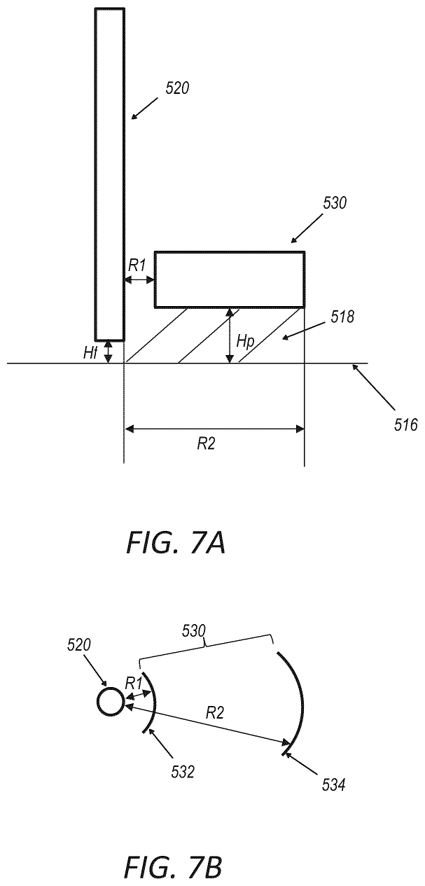

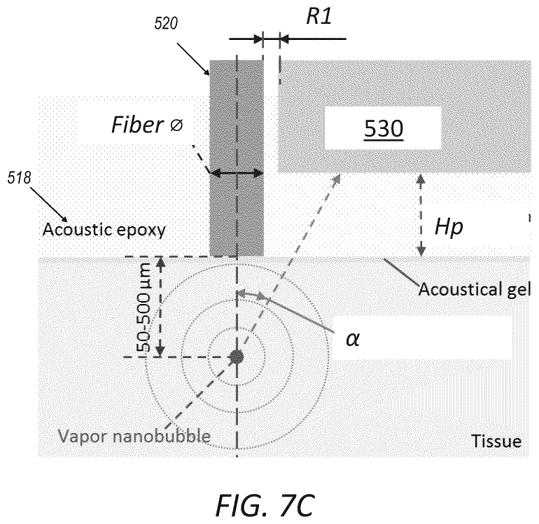

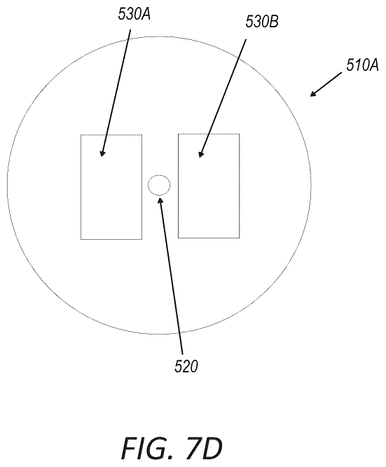

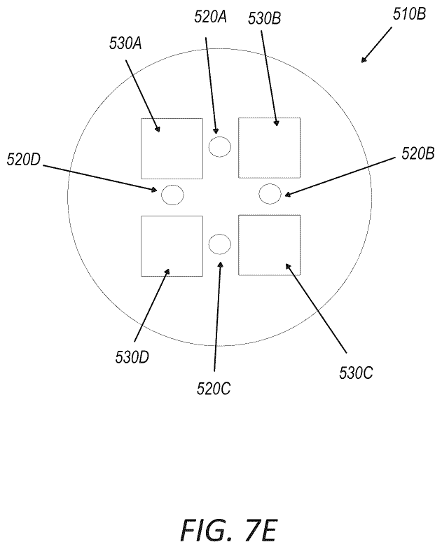

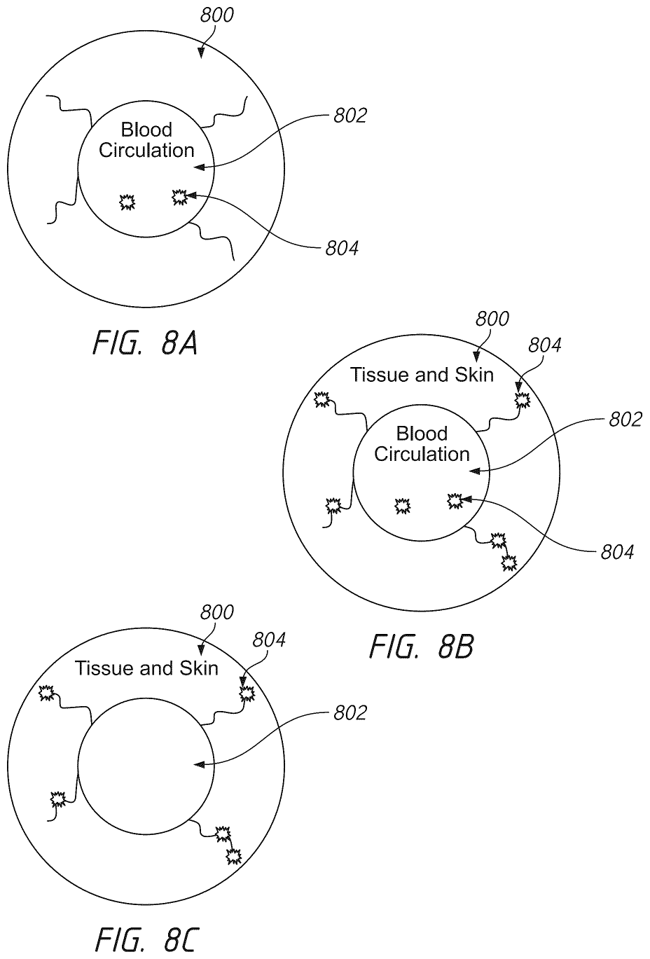

In some embodiments, the one or more processors 110 of system 10 can instruct the laser pulse generator 102 to emit a plurality of laser pulses at the location 26 to determine if there is nanobubble(s) generation at the location 26. As will be described in greater detail below, the laser pulses can have the same or different wavelengths and/or energy level. In some embodiments, the one or more processors 110 of the system 10 can instruct that the sensor probe 108 to expose to the laser pulse to different areas at the location 26. This can be achieved by mechanically scanning the surface with one optical fiber or by using multiple optical fibers (see FIG. 7E) and routing the laser pulse sequentially though various optical fibers. In some embodiments, the one or more processors 110 of the system 10 can instruct that the sensor probe 108 be moved to a second location of the test subject 20 different from the location 26 to determine if malaria-specific nanoparticles can be detected at the second location. In some embodiments, more than two fiber-acoustic detector combinations can be used (see FIG. 7E). The more than two fiber-acoustic detector combinations can be mounted in one sensor probe. Each of the more than two fiber-acoustic detector combinations can collect signals from a different area within the same or different location(s).

In some embodiments, such as illustrated in FIG. 1B, a system 12 having the same or similar features as the system 10 can include a second sensor probe 109 comprising a second optical fiber (or more than one optical fiber) 105 and a second acoustic detector (or more than one acoustic detector) 107. Features of the system 10 in FIG. 1A and the system 12 in FIG. 1B can be incorporated into one another. As shown in FIG. 1B, the first sensor probe 108 can be applied to the first location 26 and the second sensor probe 109 can be applied to a second location 28 of the test subject 20. The one or more processors 110 can be in electrical communication with the laser pulse generator 102 and both of the acoustic detectors 106, 107. The laser generator 102 can provide optical energy, such as one or more laser pulses to both of the optical fibers 104, 105. Laser pulse(s) can be directed to the first and second location 26, 28 substantially simultaneously or in succession. The one or more processors 110 can receive outputs from both of the acoustic detectors 106, 107 to determine if nanobubble generation can be detected at either or both locations. The one or more processors 110 can have a multiplex unit configured to instruct the laser pulse generator 102 to send pulses (for example, to the plurality of optical fibers in the sensor probe such as illustrated in FIG. 7E), and/or to collect signals from the acoustic detectors 106, 107. The system can also have more than two sensor probes, such as three, four, five, or six sensor probes, which can be coupled to the laser pulse generator 102 and the processors 110. The display 112 can display a single outcome of whether malaria-specific nanoparticles have been detected in the test subject 20, and/or an outcome for each location.

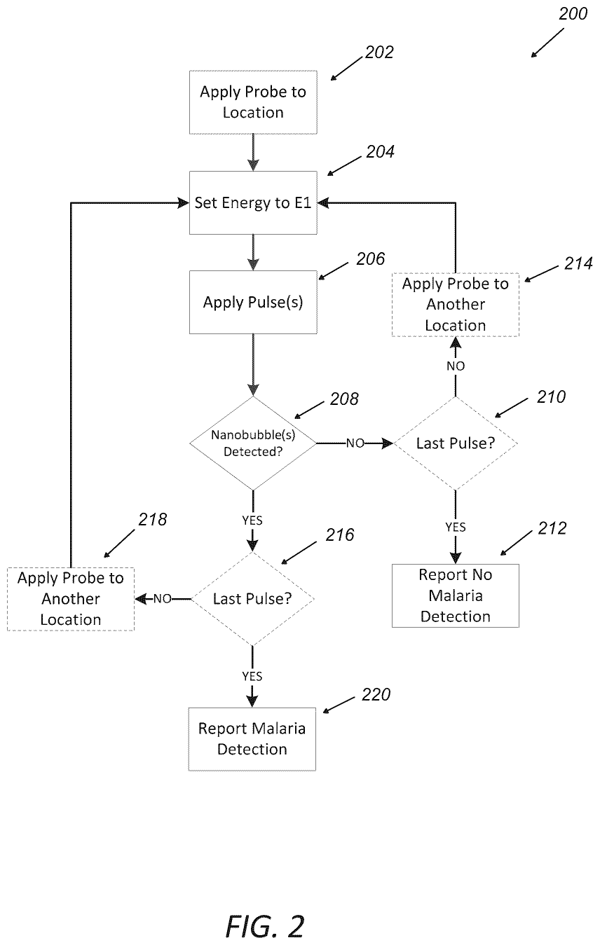

FIG. 2 illustrates an exemplary process 200 of using the system for noninvasively diagnosing and/or treating malaria, such as the system 10, 12 of FIGS. 1A and 1B. At step 202, the one or more signal processors of the system can instruct a user, such as a clinician or a health worker, to apply the malaria probe having both the optical fiber(s) and the acoustic detector(s) to a target location. The target location can be any of the test subject described above.

The step 202 can include applying a layer of optically transparent ultrasound gel (or any other material to act as an optical and acoustic coupling media between the probe and the tissue) to a probe tip surface before applying the probe to the target location. Applying the probe to the target location can include pressing the probe tip surface and/or the layer of gel firmly into contact with a surface of the target location. The layer of gel can act as an optical and/or acoustic coupler by expelling air between the probe tip surface and the surface of the target location. In some embodiment, applying the probe to the target location can also include keeping a longitudinal axis of the probe generally perpendicular to the surface of the target location. The generally perpendicular probe can prevent air from entering between the probe tip surface and the surface of the target location, which can improve optical and/or acoustic coupling of the probe and the target location.

At step 204, the one or more signal processors can set an energy level of the laser pulse generator to a first predetermined level, E1. In some embodiments, E1 can be sufficient for generating nanobubbles around malaria-specific nanoparticles up to about 0.5 mm underneath a surface of the target location. In some embodiments, E1 can have an energy level of about 1 μJ to about 50 μJ, or about 10 μJ to about 15 μJ. In some embodiment, E1 can have a pulse rate of about 1 HZ (hemozoin) to about 100 HZ (hemozoin), or about 20 HZ (hemozoin) to about 50 HZ (hemozoin), or about 20 HZ (hemozoin), or about 50 HZ (hemozoin). The one or more signal processors can set more than one energy levels, such as two different energy levels. The one or more signal processors can also set one or more than one wavelengths for the laser pulses.

At step 206, the one or more signal processors can cause the laser pulse generator to apply one or more laser pulses having an energy level of E1. The number of pulses to be applied at a location can be predetermined, manually configured, and/or determined by the one or more processors based on certain algorithms. For example, the processors can stop additional pulses as soon as nanobubble generation has been detected, or continue instructing that additional pulses be applied until a predetermined number of pulses have been applied at the location. At decision block 208, the one or more signal processors can determine based on the signals outputted by the acoustic detector if one or more nanobubbles have been generated.

If the signal is not indicative of nanobubble generation, the one or more processors can optionally determine at decision block 210 if the pulse(s) applied at the step 206 include the last or final pulse to be applied to the patient for malaria detection. The number of pulses to be applied to each patient can be predetermined, manually configured, and/or determined by the one or more processors according to certain algorithms. The pulses can be applied to one or more measurement locations on the patient. The locations can be predetermined, manually selected by a user such as a clinician, and/or determined by the one or more processors according to certain algorithms. The one or more signal processors can instruct that the same or different numbers of pulses be applied to each location.

If the pulse(s) applied at the step 206 include the last or final pulse of the process 200, the one or more signal processors can output a message that no malaria parasite is detected at step 212. The message can be an audio signal, an optical signal, a text and/or symbol displayed on a display device, or a combination thereof. If the pulse(s) applied at step 206 do not include the last or final pulse of the process 200, the one or more processors can instruct that the probe be applied to another location at step 214. Pulse(s) can be applied to the new location to determine if nanobubble(s) generation can be detected at the new location. The energy level can be the same or different for each location.

If the signal is indicative of nanobubble generation, the one or more signal processor can also optionally determine at decision block 216 if the pulse(s) applied at the step 206 include the last or final pulse to be applied to the patient for malaria detection. If the pulse(s) applied at step 206 do not include the last or final pulse of the process 200, the one or more processors can instruct that the probe be applied to another location at step 218. Whether the signal is indicative of nanobubble generation can be determined by parameters derived from an amplitude, phase and/or shape of the signal.

If the pulse(s) applied at the step 206 include the last or final pulse of the process 200, the one or more signal processors can output a message that one or more malaria parasites are detected at step 220. In some embodiments, a signal indicative of nanobubble generation can include at least one (such as, two) spikes on a time-response trace (signal) received from the acoustic detector. Time taken for detecting the spike can be used to estimate a depth of the malaria-specific nanoparticles and/or malaria parasites. Time between the two spikes can characterize the maximal size of a detected vapor nanobubble.

At the step 206, if malaria-specific nanoparticles are present at the location, the laser pulse(s) applied to the location may also be sufficient for generating nanobubbles of a size that can cause mechanical damage to the malaria parasites. In some embodiments, the nanobubbles cause mechanical damage and/or destruction of the malaria parasites without harming uninfected blood cells and/or tissues.

If some individual signals obtained from healthy tissue look similar to nanobubble-specific signals associated with malaria disease and presence of HZ (HEMOZOIN) in the laser-exposed volume, groups of N signals (N ranges from 1 to 10,000), each in response to the corresponding laser pulse, can be analyzed statistically. Examples of statistical analyses can include using signal amplitude-derived diagnostic parameters, such as the normalized positive count, N, and the hemozoin index, HI, and a user-defined diagnostic threshold for the N and HI parameters. The normalized positive count, N, can be calculated using the formula N=Nₚ/Nₜ, where Nₜ is the total number of the collected signals, Nₚ is the number of signals with the peak-to-peak amplitude above a threshold T. The hemozoin index, HI, can be calculated using the formula HI=<A>−T*NNPCT, where <A> is an average peak-to-peak amplitude of the signals above T. Parameters above the diagnostic threshold would be indicative of malaria disease and parameters below the threshold would be indicative of healthy condition. In some embodiments, the hemozoin index of a malaria-positive signal can be about one order of magnitude greater than the hemozoin index of a malaria-negative signal. Examples of statistical analyses can also include a peak time-delay parameter and a user-defined diagnostic threshold for the time-delay. More details of the statistical analyses are described further below.

Additional diagnostic combinations in addition to applying one level of the laser pulse energy, one laser wavelength, and/or one type of the acoustic detector can be used to further improve the sensitivity and specificity of the detection of malaria-specific signal in the background of the bulk signal associated with healthy (malaria-negative) tissue.

In some embodiments, the process 200 can be repeated with a different laser wavelength than the wavelength used in the previous process 200. One of the two different wavelengths can be associated with a maximal optical absorption by malaria-specific nanoparticles.

In some embodiments, the process 200 can be repeated with a different laser pulse energy level than the laser pulse energy level used in the previous process 200. The difference in the nanobubble signals detected in the two processes 200 can be different due to the different number of bulk signals produced by the different energy levels.

The process 200 can be repeated with two different laser wavelengths and/or two different laser pulse energies, using the same sensor, or different optical fiber-acoustic detector combinations in the same sensor (which will be described below with reference to FIG. 7E).

The process 200 can also additionally and/or alternatively be performed with a sensor have two or more different acoustic detectors (described below with reference to FIGS. 7D and 7E). The different acoustic detectors can have different acoustic properties, which can enable detection of the acoustic signal in different frequency domains and can improve the identification of the malaria-specific signal and/or nanobubble generation.

As the malaria diagnosis and/or treatment apparatuses and processes disclosed herein are based on photo-excitation of the malaria-specific nanoparticle, the process 200 can be reproducible and free from parasite resistance. As the generation of laser pulses and generation of nanobubbles take seconds, or at most several minutes, the treatment process 200 can be more efficient than traditional malaria diagnosis and/or treatment procedures.

### Examples of a Malaria Probe

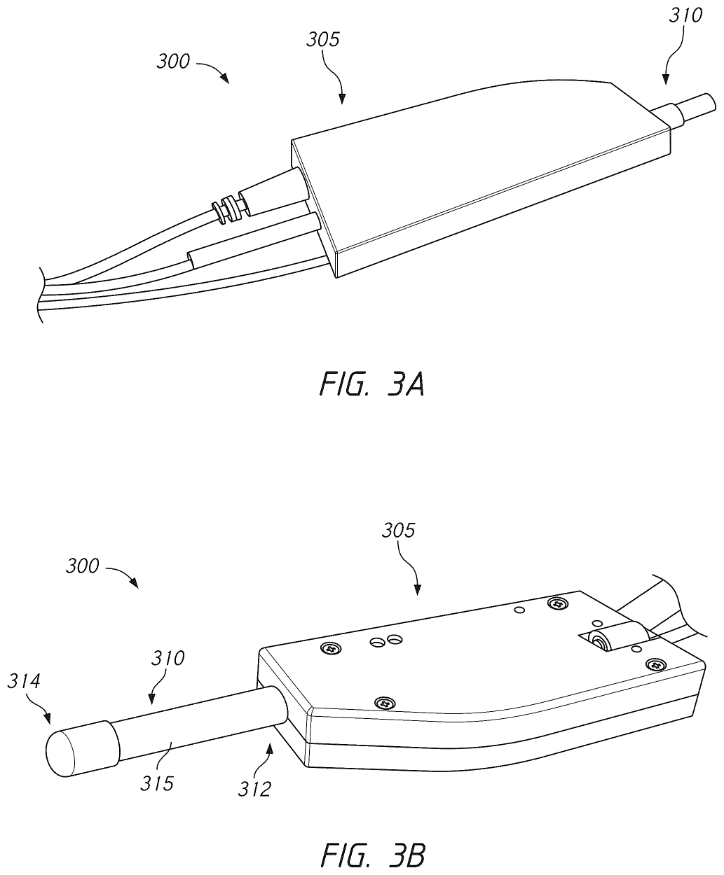

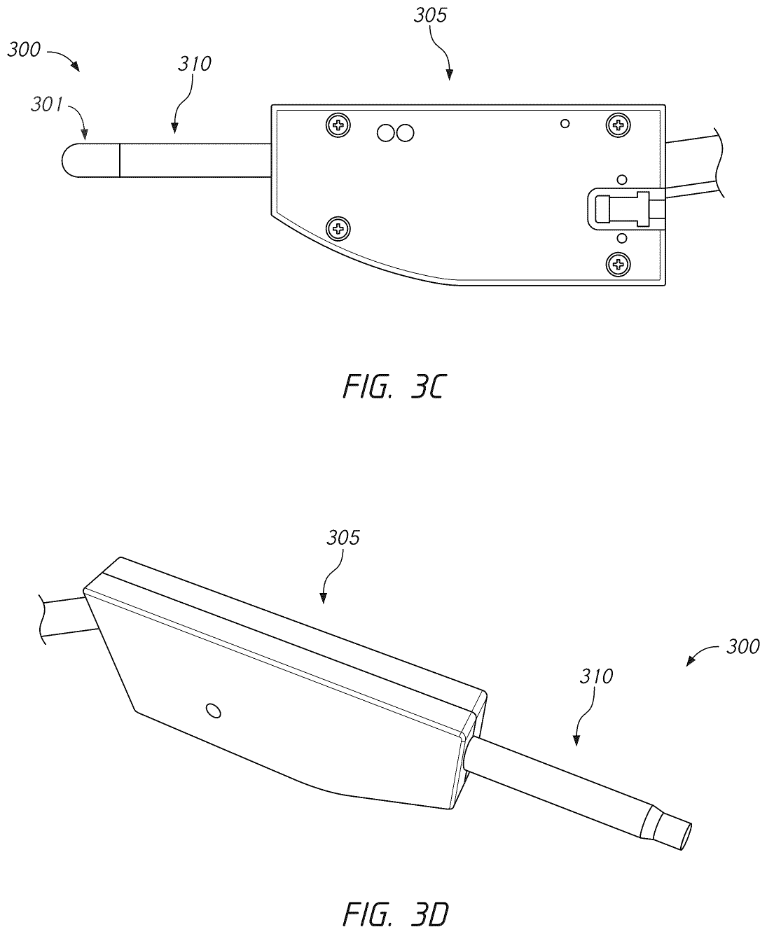

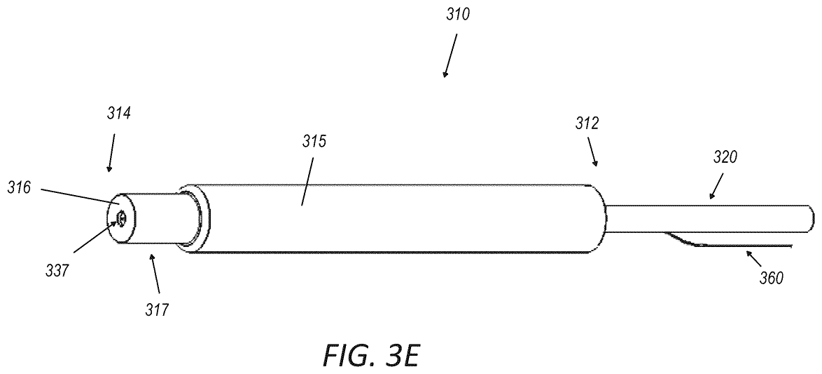

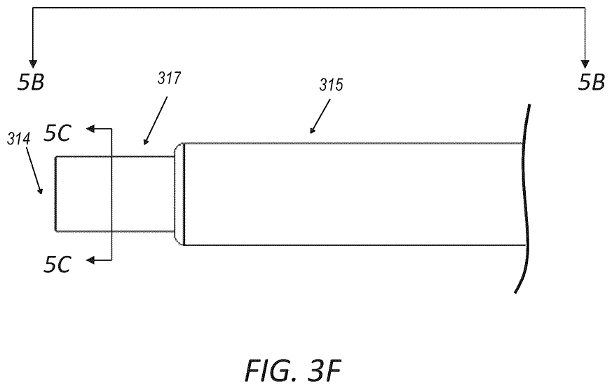

Examples of the malaria diagnosis and/or treatment probe will now be described. As shown in FIGS. 3A-3C, the probe 300 can have a sensor portion 310 and a body portion 305. The sensor portion 310 can have a proximal end 312 and a distal end 314. The proximal end 312 of the sensor portion 310 can be coupled to the body portion 305. The sensor portion 310 can extend distally from the body portion 305. The sensor portion 310 can have a probe housing 315 enclosing the optical fiber and the acoustic detector. The body portion 305 can include circuitry and/or processors for driving the optical fiber and/or the acoustic detector. In FIGS. 3B and 3C, the distal end 314 of the sensor portion 310 is covered by a protective cap 301 when the probe 300 is not in use. FIG. 3D illustrates the distal end of the sensor portion with the protective cap removed.

FIGS. 3E and 3F illustrate the sensor portion 310 of the probe 300 with the protective cap 301 removed. As shown in FIGS. 3E and 3F, the distal end 314 of the sensor portion 310 can have a sensor tip cover 317 extending distally from the probe housing 315. The sensor tip cover 317 can have a smaller outer diameter than the probe housing 315. The sensor tip cover 317 can include a generally centrally located opening 337. The optical fiber and the acoustic detector can be located generally concentrically with the opening 317. The probe 300 can also include electrical connections, such as lead wire 360, for connecting the acoustic detector 330 with a signal processor. The sensor design shown in FIGS. 3A, 3E, and 3F may be more prone to detecting false-positive signals, reduced sensitivity to the detection of true-positive signals, which may be ameliorated in future sensor designs.

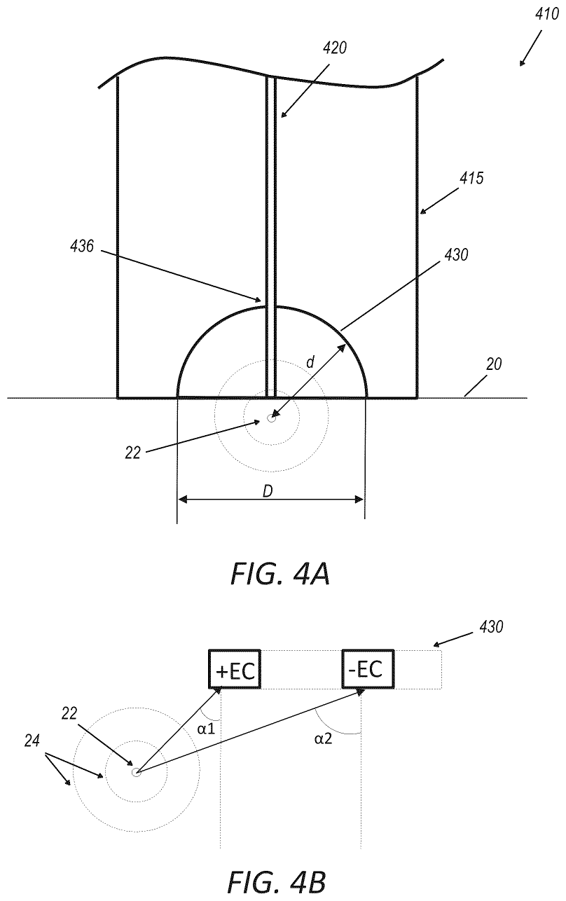

Before describing the sensor portion 310 in greater detail, a configuration of the malaria probe sensor portion 410 known in the art is described with reference to the schematic drawing in FIG. 4A. The sensor portion 410 can have an optical fiber 420 and a spherical acoustic detector 430 enclosed by the probe housing 415 and/or the sensor tip cover such as shown in FIGS. 3D and 3E. The optical fiber 420 can extend substantially along a central longitudinal axis of the sensor portion 410.

The acoustic detector 430 can be generally a hemisphere with a substantially centrally located opening 436 to accommodate the optical fiber 420. The spherical acoustic detector 430 can circumferentially surround the optical fiber 420. In some instances, the spherical acoustic detector 430 can have a diameter D of about 4 mm. When a malaria-specific nanoparticles-generated nanobubble 22 (“nanobubble”) is generated under the surface of the target location 20, a distance d between the spherical acoustic detector 430 and the nanobubble 22 can be between about 2 mm to about 3 mm, or greater.

The spherical acoustic detector 430 can have some disadvantages. The pressure amplitude for the spherical pulse drops inversely proportional to the square of the distance from the source, as described above. As a result, the distance d can be large enough to result in substantial loss of the pressure between the nanobubble 22 and the acoustic detector 430 and hence in the reduced sensitivity of such sensor for detecting nanobubbles.

The acoustic detector 430 can be one or more flat piezo elements, which can be cheaper than spherical piezo elements. If the flat piezo elements are located in the same distance as the spherical piezo elements as shown in FIG. 4A, it may be necessary to use a flat piezo element with a large surface area to detect spherical pulses with reduced pressure amplitude at the distance d. However, a flat piezo element having a large detector surface area can result in a weaker signal output. As shown in FIG. 4B, due to the large surface area of the acoustic detector 430, the pressure pulse 24 generated by the nanobubble 22 can strike the surface of the acoustic detector 430 at different angles of incidence and be in different phases. Some of the pressure pulses, such as the pressure pulses hitting the surface of the piezo element at an angle of incidence a1, can generate a positive electrical charge. Some of the pressure pulses, such as the pressure pulses hitting the surface of the piezo element at an angle of incidence a2, can generate a negative electrical charge. Such dephasing of piezo-effect in piezo element can result in the loss of the sensitivity because positive and negative charges can cancel each other. As a result, a greater surface area of the piezo element can reduce, not increase, the sensor sensitivity.

Further challenges of using the spherical piezo element 430 can include the high cost of spherical piezo elements, and/or the complexity of incorporating the spherical piezo element 430 and the optical fiber 420 into the single probe. In some instances, a separate holder is required to hold the optical fiber 420 in the opening 436 of the spherical piezo element 430. The cost and complexity can make it difficult to mass-produce or commercialize a probe having a sensor portion 410.

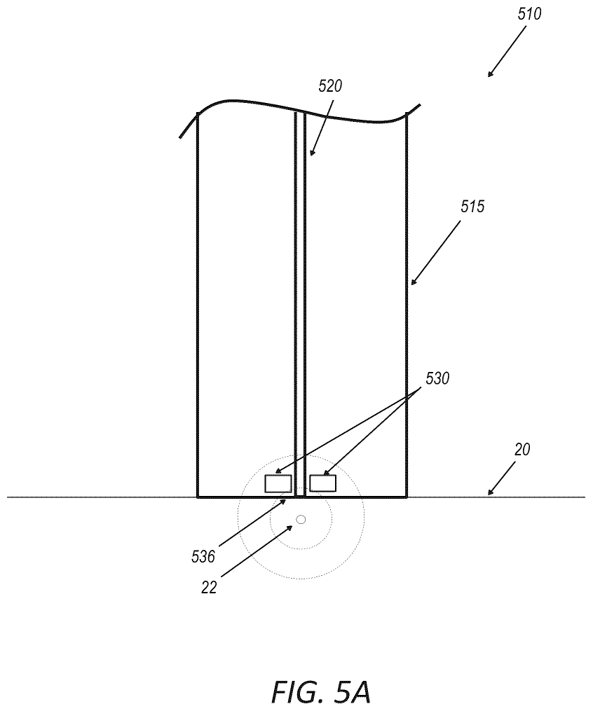

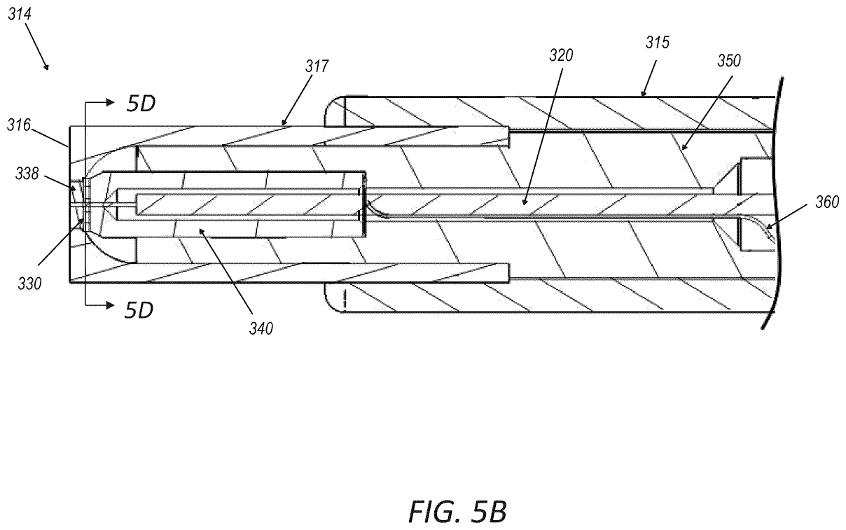

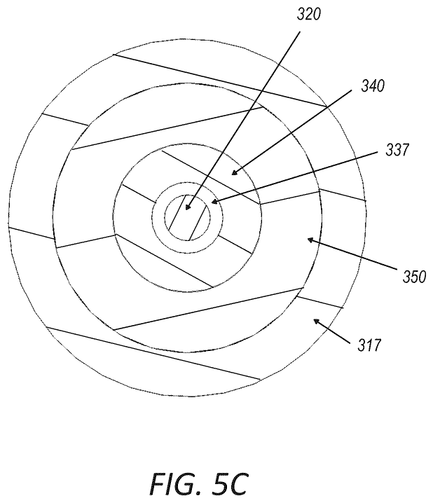

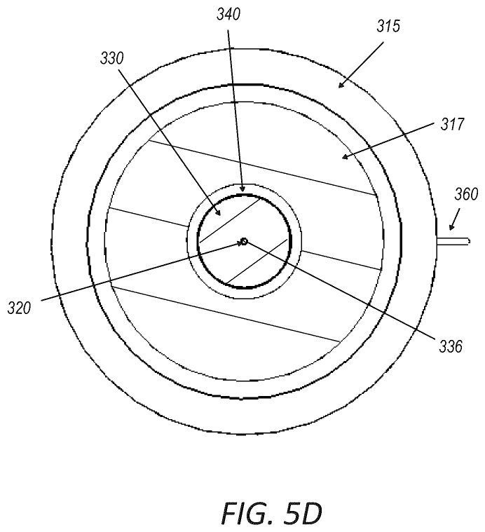

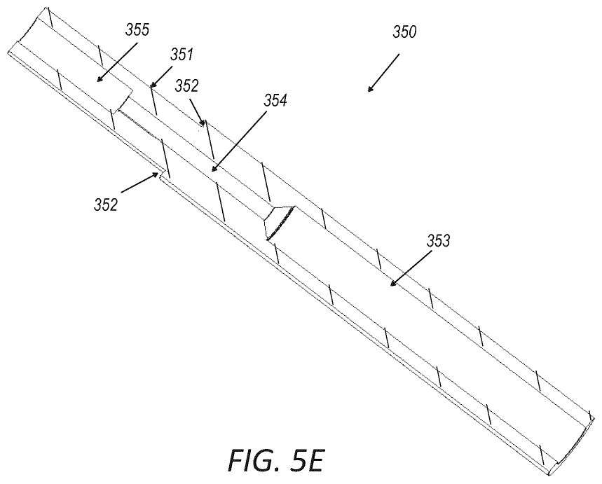

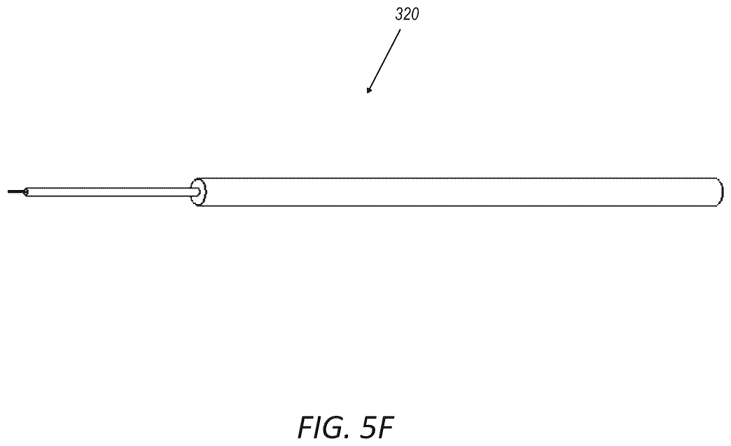

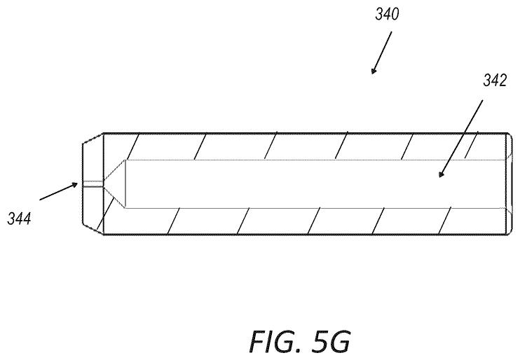

FIG. 5A illustrates schematically an example malaria probe sensor portion 510 that overcomes some challenges of using the spherical and/or flat acoustic detector 430, and/or other challenges. The sensor portion 510 can have an optical fiber 520 and a substantially flat acoustic detector 530 enclosed by the probe housing 515 and/or the sensor tip cover such as shown in FIGS. 3E and 3F. As noted above, this sensor design may be more prone to detecting false-positive signals, reduced sensitivity to the detection of true-positive signals, which may be ameliorated in future sensor designs.

The optical fiber 520 can extend substantially along a central longitudinal axis of the sensor portion 510. The optical fiber 520 can have a core diameter, or optical aperture, in the range of about 50 μm to about 200 μm, or about 80 μm to about 150 μm, or about 100 μm. The core diameter of the optical fiber 520 is small enough to reduce background bulk optical absorbance in order to maintain sufficient signal-to-noise ratio, and is not too small so it can easily miss the malaria parasite or malaria-specific nanoparticle.

The substantially flat acoustic detector 530 can form a disc with a substantially centrally located opening 536 to accommodate the optical fiber 520. The flat acoustic detector 530 can circumferentially surround the optical fiber 520. In some embodiments, the flat acoustic detector 530 can hold the optical fiber 520 in place without a separate holder. The flat acoustic detector 530 can be a piezo element. The piezo element can comprise a navy type II material, navy type VI material, any other piezo materials, or any combination of piezo and other materials.

FIG. 5B illustrates a longitudinal cross-section of a distal part of the sensor portion 310 of the probe 300 in FIG. 3F, which incorporates the optical fiber 320 circumferentially surrounded by a flat acoustic detector 330. Accordingly, the sensor portion 310 can be an example of the sensor portion 510 in FIG. 5A. As shown in FIG. 5B, a proximal portion of the sensor tip cover 317 can be received in and/or securely attached (for example, by an interference fit, adhesives, welding, and the like) to a lumen of the probe housing 315. A proximal end of the sensor tip cover 317 can terminate near or abut a shoulder 352 (see FIG. 5E) of a fiber housing 350.

As shown in FIG. 5E, the fiber housing 350 can have a distal portion 351 transitioning to a proximal portion 353 the shoulder 352. The distal portion 351 can have a first external diameter that is smaller than a second external diameter of the proximal portion 353. The second external diameter can be sized to allow the proximal portion 351 to be slidably received in the lumen of the probe housing 315. As shown in FIG. 5B, gap between an inner wall of the probe housing 315 and an outer wall of the distal portion 351 of the fiber housing 350 can accommodate a wall thickness of the sensor tip cover 317.

Also as shown in FIG. 5E, the fiber housing 350 can have a lumen with varying internal diameters. The internal diameters of the lumen proximal 353 and distal 355 to a neck 354 can be greater the internal diameter of the neck 354. The lumen of the fiber housing 350 can accommodate and/or support the optical fiber 320 (see FIG. 5F), which can also have varying diameters. The variation in the outer diameter of the optical fiber 320 can be due to a varying thickness in a coating or protective layer(s) circumferentially surrounding the optical fiber 520.

As shown in FIG. 5B, the internal diameter of the neck 354 can reduce movements of the optical fiber 320 within the housing 315. As shown in FIGS. 5B and 5C, the lumen distal 355 to the neck 354 can also accommodate a support body or ferrule 340. The support body 340 can include a lumen 342 to accommodate the optical fiber 320. The internal diameter of the lumen 342 can reduce movements of the optical fiber 320 within the housing 315 and/or the sensor tip cover 317. The support body 340 can also improve rigidity of the distal end of the sensor portion 310. The lumen 342 can terminate at a bore 344, which can have a reduced internal diameter compared to the internal diameter of the lumen 342. A distal portion of the optical fiber 320 that is not covered by coating or protective layer(s) (see FIG. 5F) can extend through the bore 344.

As shown in FIGS. 5B and 5D, the flat acoustic detector 330 can be located distal of the support body 340 and enclosed by the sensor tip cover 317. The acoustic detector 330 can have a generally centrally located opening 336 for accommodating and/or supporting a distal end of the optical fiber 320.

As shown in FIG. 5B, the sensor portion 310 can also include a front layer 318. The front layer 318 can be substantially flush with a distal surface of the sensor tip cover 317 to define a probe tip surface 316. The front layer 318 can improve acoustic coupling between the acoustic impedance of the tissue of the target location and the acoustic detector 330, to optimize response of the acoustic detector 330, to protect the acoustic detector 330 from backscattered laser radiation, and/or to form a durable contact surface which protects the acoustic detector 330 from mechanical damage, such as scratches. The front player 318 can include a material having suitable speed of sound, optical transparency, and/or optical absorbance or scattering properties. The speed of sound in the material can be similar to that in water. The material may not be transparent as a transparent layer can deliver a portion of the backscattered laser pulses into the acoustic detector 330 and thus create a noise in the signal. The material also may have minimal optical absorbance to avoid a photoacoustic effect in the front layer caused by the backscattered laser pulses. In some embodiments, the front layer 318 can have acoustic epoxy and metal particles that scatter backscattered laser radiation and thus shield piezo element from such backscattered laser radiation.

Although the acoustic detectors 330, 530 are illustrated as a single flat element, the acoustic detector can also include two flat discs 530A, 530B, located on substantially opposite sides of the optical fiber 520, such as shown in FIG. 7D. The two flat discs 530A, 530B can have the same or different acoustic properties, such as the frequency spectrum. For example, the disc 530A can have a high-frequency piezo material and the disc 530B can have a low-frequency piezo material. The discs 530A, 530B can each detect amplitude and time information of the acoustic signal in different frequency spectra. The low frequency disc can operate in the range of about 0.01 MHZ (hemozoin) to about 4 MHZ (hemozoin). The high frequency disc 530B can operate in the range of about 4 MHZ (hemozoin) to about 100 MHZ (hemozoin).

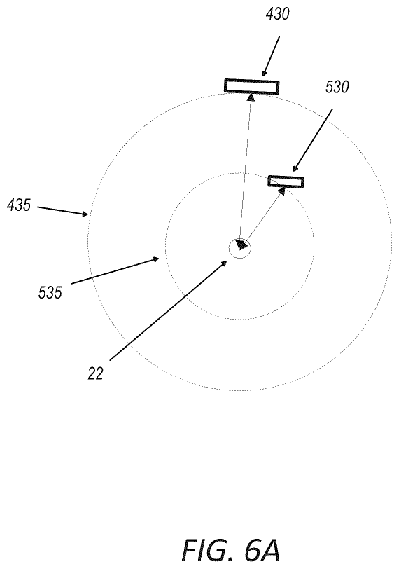

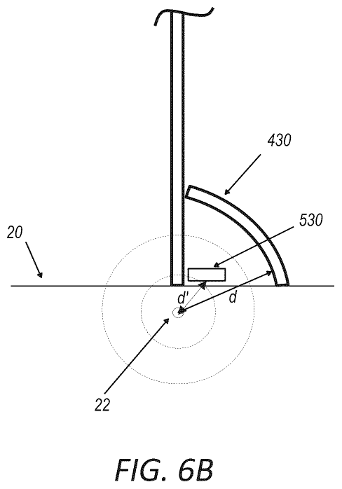

FIG. 6A illustrates a comparison of an acoustic detector 530 (which can also be the acoustic detector 330) at a location 535 that is closer to the nanobubble 22 than an acoustic detector 430 at a location 435. As shown in FIG. 6A, a spherical pressure pulse can arrive at the acoustic detector 430 and the acoustic detector 530 at different angles of incidence. As the amplitude of spherical pulse is inversely proportional to the square of the distance from the source, the acoustic detector 530 can detect a stronger acoustic pulse from generation of the nanobubble 22 than the detector 430. Similarly, as shown in FIG. 6B, the flat acoustic detector 530 can be brought closer to the nanobubble 22 than the spherical acoustic detector 430. In some embodiments, the distance d′ between the flat acoustic detector 530 and the nanobubble 22 can be as small as about 0.1 mm or about 0.2 mm, compared to about 2 mm to about 6 mm between the spherical acoustic detector 430 and the nanobubble 22. The flat acoustic detector 530 can also be smaller than the spherical acoustic detector 520. In some embodiments, such as shown in FIGS. 7A and 7B, the flat acoustic detector 530 can have an external diameter, or acoustic aperture, of about 0.2 mm to about 3 mm, or about 1 mm to about 2 mm, compared to about 4 mm to 8 mm for the spherical acoustic detector 430. A distance R2 between the outer wall of the optical fiber 520 and a radially external edge 534 of the acoustic detector 530 can be no greater than about 0.3 mm to about 1.5 mm. As described above, the small R2 can contribute to reduction of de-phasing of the pressure-to-charge conversion process in a piezo element.

As shown in FIGS. 7A-7C (which illustrate a preferred sensor probe configuration), a gap R1 between an outer wall of the optical fiber 520 and a radially internal edge 532 of the acoustic detector 530 can be about 0.01 mm to about 0.03 mm, or about 0.02 mm. A small gap R1 can bring the acoustic detector 530 closer to a position with the minimal angle of acoustic incidence and hence the maximal sensitivity of the sensor. This is because pressure pulses striking the surface of an acoustic detector, such as a piezo element, at a smaller angle of incidence can generate stronger signals than pressure pulses striking the surface of the acoustic detector at a larger angle of incidence. The angle of incidence, a, at which the pressure pulses strikes the surface of the acoustic detector can be less than about 45°. This can be due to flat piezo elements usually having relatively narrow pointing diagram, which can become narrower with the increasing size of piezo element. The optical fiber 520 can have a diameter in the range of about 10 μm to about 400 μm, or about 50 μm to about 200 μm, or about 80 m to about 150 μm, or about 100 μm.

Although a flat acoustic detector can be inferior, in theory, to a spherical detector for detecting the spherical pressure pulses from the nanobubble, the flat acoustic detector 530 is small in its size and in close proximity to the nanobubble such that the effect of the flat shape on the signal can be small. As a result, the flat acoustic detector 530 can improve the sensitivity and selectivity of malaria detection.

Moreover, the flat acoustic detector 530 does not have some of the above-described disadvantages associated with the spherical acoustic detector 430. When spherical pressure pulses arrive at the acoustic detector 530 having a small surface area and located in close proximity to the point source, the pressure pulses can arrive at the surface of the acoustic detector 530 at substantially the same or similar angles of incidence. As a result, it is less likely that opposite electrical charges are produced by the pressure pulses and the effect of de-phasing can be reduced and/or minimized. The flat acoustic detector 530 can thus improve the peak-to-peak amplitude of the spike. Larger peak-to-peak amplitude of the spike can improve the sensitivity and/or specificity of the sensor portion 510.

Incorporating the flat acoustic detector 530 and the optical fiber 520 into a single probe can be less complex than incorporating the spherical acoustic detector 430 and the optical fiber 420 into a single probe. As described above, the flat acoustic detector 530 can directly hold the optical fiber 520 without a separate holder. A flat acoustic detector, such as a flat piezo element, can also be significantly cheaper than a spherical acoustic detector, such as a spherical piezo element. As a result, the sensor portion 510 can be more suitable for mass production than the sensor portion 410.

With continued reference to FIG. 7A, the optical fiber 520 can terminate at or substantially at a probe tip surface 516 so that a distance Hf between the tip of the optical fiber 520 and the probe tip surface 516 can be substantially zero. Any material between the optical fiber tip and the surface of the target location can cause a background residual due to residual optical absorption, even if the material is transparent and the residual optical absorption is minor. Having the optical fiber 502 terminating at the probe tip surface 516 can reduce background residual and/or bring the optical source as close to the nanobubble as possible. As shown in FIG. 7C, having the optical fiber 5 terminating at the probe tip surface can bring the optical source closer to the malaria-specific nanoparticle. A distance between a distal end of the optical fiber 520 and the malaria-specific nanoparticle can be between about 0.05 mm to about 0.5 mm.

As shown in FIGS. 7A and 7C, the acoustic detector 530 can be recessed from the probe tip surface 516 by a gap Hp. Hp can be from about 0.02 mm to about 4 mm, about 0.02 mm to about 1 mm, or from about 0.1 mm to about 0.2 mm. A distal portion of the acoustic detector 530 and the optical fiber 520 can be embedded in a front layer 518. The front layer 518 can improve acoustic coupling between the acoustic impedance of the tissue of the target location and the acoustic detector 530, to optimize response of the acoustic detector 530, to protect the acoustic detector 530 from backscattered laser radiation, and/or to form a durable contact surface which protects the acoustic detector 530 from mechanical damage, such as scratches. The front player 518 can comprise a material having suitable speed of sound, optical transparency, and/or optical absorbance or scattering properties. The speed of sound in the material can be similar to that in water. The material may not be transparent as a transparent layer can deliver a portion of the backscattered laser pulses into the acoustic detector 530 and thus create a noise in the signal. The material also may have minimal optical absorbance to avoid a photoacoustic effect in the front layer caused by the backscattered laser pulses. In some embodiments, the front layer 518, such as shown in FIG. 7C, can comprise acoustic epoxy. The front layer 518 can also include metal particles in the acoustic epoxy to scatter backscattered laser radiation and thus shield piezo element from such backscattered laser radiation.

FIGS. 7D and 7E illustrate schematically malaria sensor probe portions 510A, 510B, which can have any of features of the malaria sensor probe portions described with reference to FIGS. 7A-7C, including but not limited to the dimensions and relative positions of the optical fiber, acoustic detector, and/or probe tip surface. As described above, FIG. 7D illustrates a malaria probe sensor portion 510A with an optical fiber flanged by two flat acoustic detectors 530A, 530B. As shown in FIG. 7E, more than two optical fiber-acoustic detector combinations can be mounted in a malaria probe sensor portion 510B. The sensor portion 510B can include a plurality of optical fibers 520A, 520B, 520C, 520D, and a plurality of acoustic detectors 530A, 530B, 530C, 530D. Each one of the plurality of optical fibers 520A, 520B, 520C, 520D can be located between two of the plurality of acoustic detectors 530A, 530B, 530C, 530D. Example combinations of optical fiber-acoustic detector can include the combination of optical fiber 520A and acoustic detectors 530A, 530B, the combination of optical fiber 520B and acoustic detectors 530B, 530C, the combination of optical fiber 520C and acoustic detectors 530C, 530D, and the combination of optical fiber 520D and acoustic detectors 530A, 530D. Each of the optical fiber-acoustic detector combinations can collect signals from a different area within the same or different measurement location. The plurality of acoustic detectors can have the same or different acoustic properties as disclosed herein. As noted above, this sensor design may be more prone to detecting false-positive signals, reduced sensitivity to the detection of true-positive signals, which may be ameliorated in future sensor designs.

In some embodiments, the sensor portion 510 can be reusable. A user can wipe the probe tip with alcohol to sterilize or disinfect the probe tip after each patient. In some embodiments, a disposable cap made of a thin film can be applied to the probe tip for each patient. The thin film can have little impact on the optical and acoustic contact between the sensor and the target location. The disposable cap can save a user's time for sterilizing or disinfecting the probe tip after each patient. In some embodiments, the film can have a thickness of about 1 μm to about 50 μm, or about 1 μm to about 20 m. The film may be covered with ultrasound gel or other biologically safe and optically- and acoustically-coupling material. The sensor probes described herein and a predetermined number of caps can also be provided in a kit. The number of caps can be sufficient for the life time and/or anticipated life time of the sensor probe in the kit.

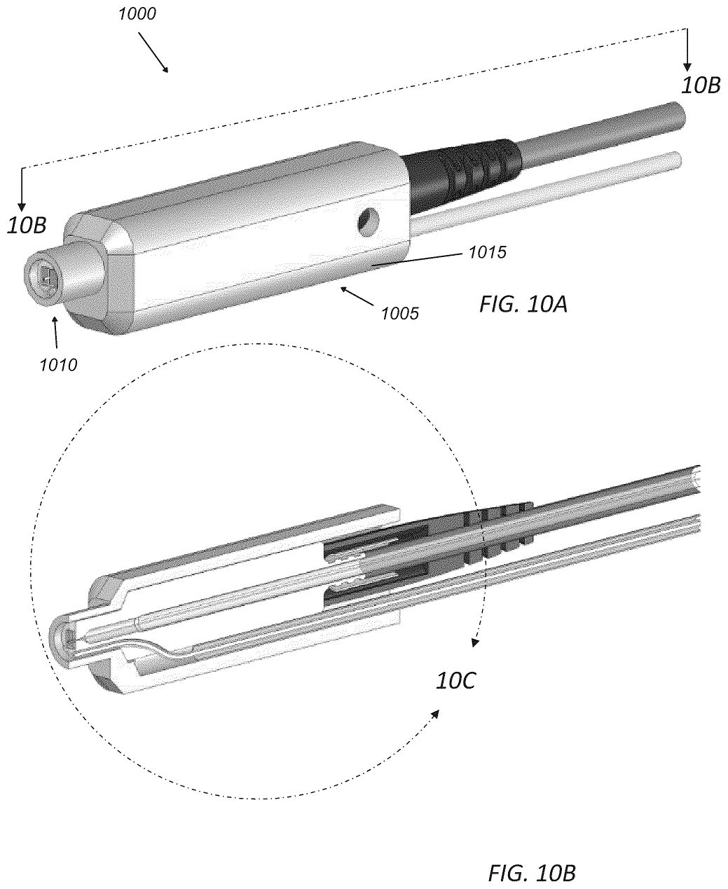

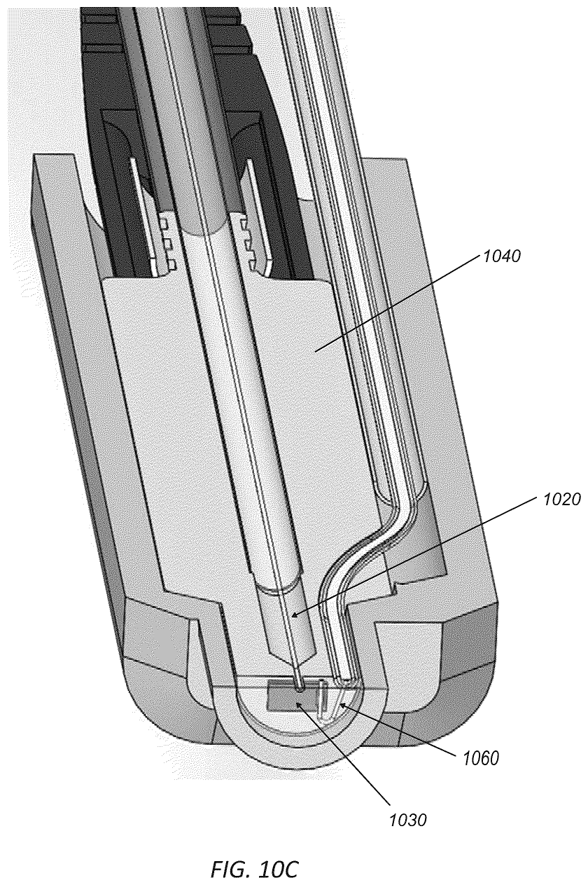

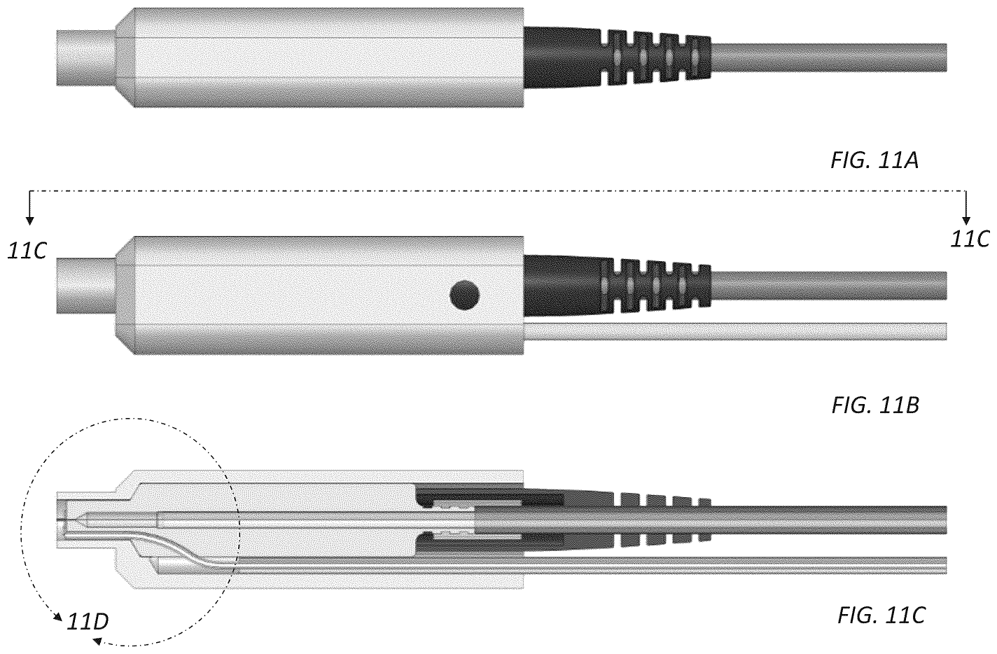

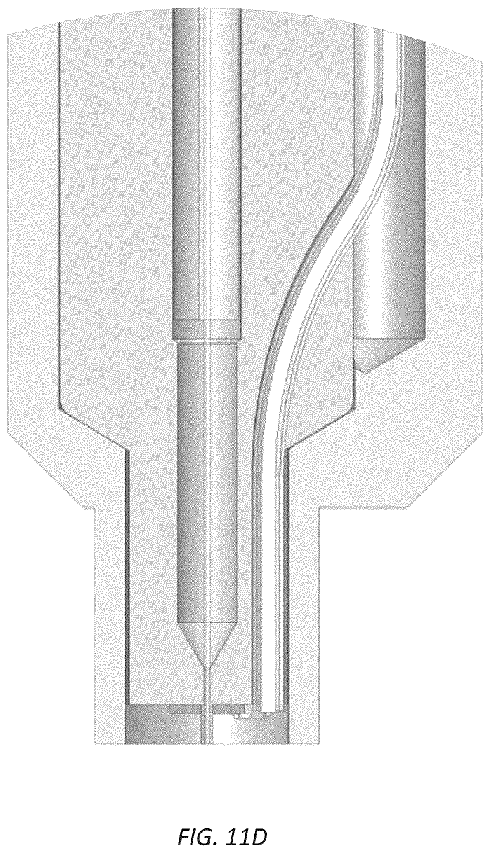

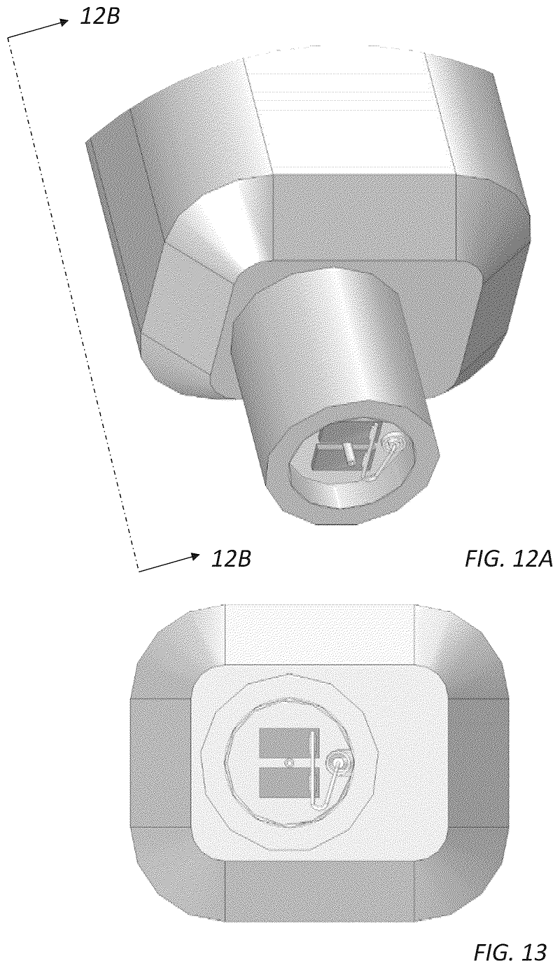

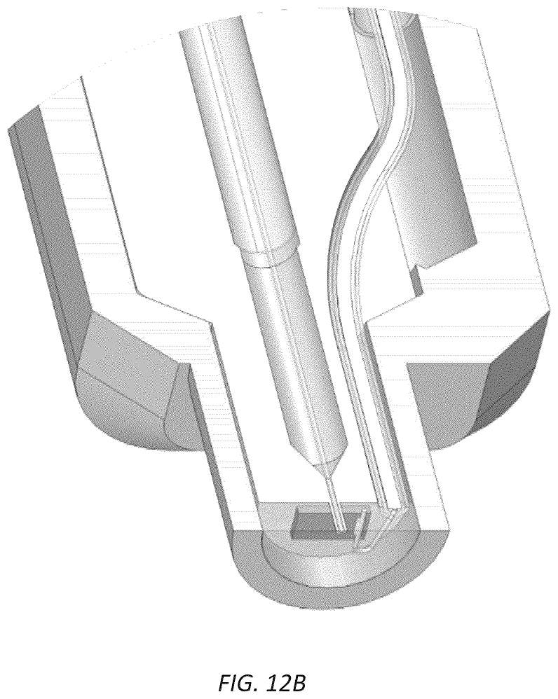

FIGS. 10A-13 illustrate an example malaria probe 1000. This sensor design may be more prone to detecting false-positive signals, reduced sensitivity to the detection of true-positive signals, which may be ameliorated in future sensor designs. The probe 1000 can have the same or similar features as the probe 500. Features of the probe 500 and the probe 1000 can be incorporated into one another.

The probe 1000 can have a sensor portion 1010 and a body portion 1005. The sensor portion 1010 can be coupled to the body portion 1005 and can extend distally from the body portion 1005. The sensor portion 1010 can have a smaller outer dimension than the body portion 1005. The probe 1000 can have a probe housing 1015 enclosing an optical fiber 1020 and a substantially flat acoustic detector 1030.

The optical fiber 1020 can extend substantially along a central longitudinal axis of the sensor portion 1010. The substantially acoustic detector 1030 can comprise two or more flat elements (such as discs) placed next to each other with the optical fiber 1020 running through a gap between the elements. As described above with reference to FIG. 7D, the two or more flat elements can have the same or different acoustic properties. The acoustic detector 1030 can be held in place inside the sensor portion 1010 by a support body 1040. The support body 1040 can have a lumen to accommodate the optical fiber 1020. The probe 1000 can also include electrical connections 1060 for connecting the acoustic detector 1030 with a signal processor.

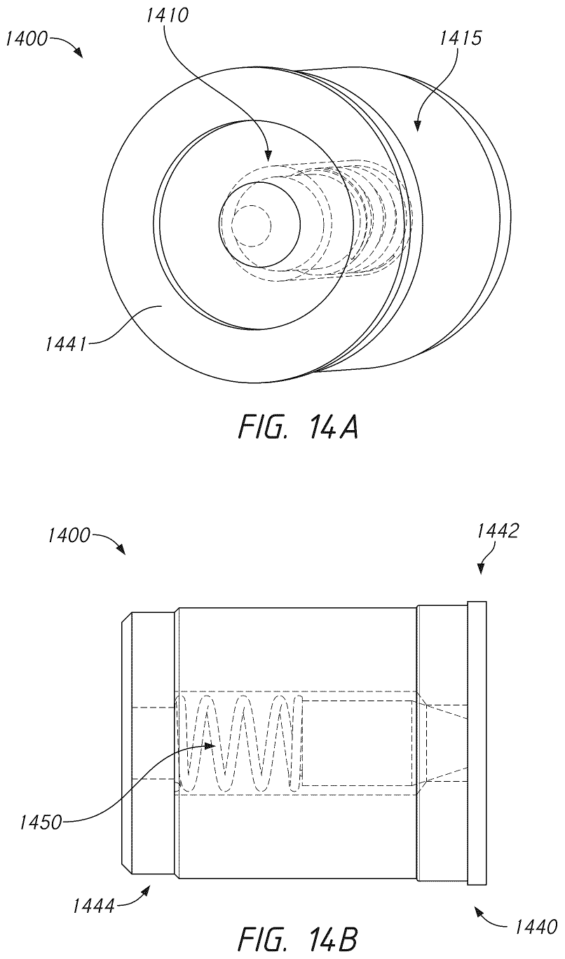

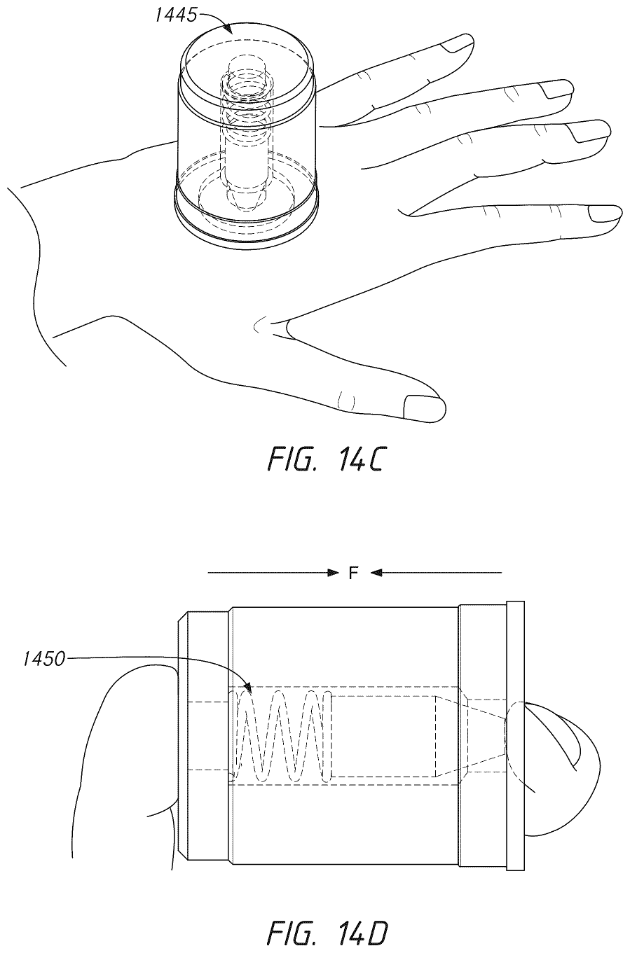

FIGS. 14A-14D illustrate an example malaria probe 1400. The probe 1400 can have any of features of the probe 500, 1000. Accordingly, features of the probe 500, features of the probe 1000, and features of the probe 1400 can be incorporated into one another. Although the probe 1400 is illustrated as having a generally cylindrical shape, the shape of the probe 1400 is not limiting. Other physical specifications of the probe 1400, such as size and materials, are also not limiting.

The probe 1400 can have a sensor portion 1410 and a housing 1415. The sensor portion 1410 can include an optical source, such as an optical fiber (or more than one optical fiber) coupled to a laser pulse generator, and an acoustic detector (or more than one acoustic detector), such as a piezo element. The optical source can be configured to generate laser pulses of at least one (one, two, or more) predetermined energy levels and/or wavelengths. The optical fiber can terminate at or near the patient interface 1442. The laser pulses can be configured to generate one or more nanobubbles around malaria-specific nanoparticles that are under a surface of a measurement site. The measurement site can be on a patient's body, or a flow cuvette described below. The acoustic detector can be configured to detect acoustic pulses generated by the one or more nanobubbles and output a signal indicative of the detected acoustic pulses to at least one signal processor. The acoustic detector can be substantially flat and in close proximity with the optical source.

The sensor portion 1410 can be located at least partially within the housing 1415. The housing 1415 can include a distal end 1440 and a proximal end 1444. At the distal end 1440, the housing 1415 can include a mounting component 1442. The mounting component 1442 can provide a greater footprint of the probe 1400 on the measurement site, such as the patient's skin, than the contact between the sensor portion 1410 and the patient's skin. In the illustrated embodiment, the mounting component 1442 can be generally ring-shaped to accommodate the sensor portion 1410 located generally at the center of the patient interface 1442.

The mounting component 1442 can include a patient attachment mechanism, which can have an adhesive layer, a gel layer, and/or otherwise. The mounting component 1442 can also include an adhesive layer and/or gel layer that is covered by a liner 1441 when the probe 1400 is not in use. When in use, such as shown in FIG. 14C, the liner 1441 can be removed to expose the adhesive layer on the mounting component 1442. The adhesive layer can improve and/or maintain contact between the probe 1400 and the measurement site. A firmer contact between the sensor portion 1410 of the probe 1400 and the measurement site can improve optical and acoustic coupling between the sensor portion 1410 and the patient's tissue, and/or detection of the acoustic pulses by the acoustic detector of the probe 1400.

The probe 1400 can be coupled electrically to the laser pulse generator, the signal processor, and/or the display that are disclosed herein. The housing 1415 of the probe 1400 can have an opening 1445 (shown in FIG. 14C) at the proximal end 1444. The sensor portion 1410 can establish communication with the laser pulse generator, the signal processor, and/or the display, via one or more cables extending into the housing 1415 through the opening 1445.

As shown in FIGS. 14B and 14D, the housing 1415 can have a length that is greater than a length of the sensor portion 1410. A spring 1450 can be positioned between the proximal end 1440 of the housing and a proximal end of the sensor portion 1410. The spring 1450 can be configured to bias the sensor portion 1410 distally such that a distal end of the sensor portion 1410 can be at or near the distal end 1440 of the housing 1415. The distal end of the sensor portion 1410 can optionally be recessed from the distal end of the housing 1415 when the sensor portion 1410 is biased to a distalmost position by the spring 1450. The recess can allow the housing 1415 to shield the sensor portion 1410 from mechanical impacts, such as during a fall. When the measurement site is on the patient's body, the skin at the measurement site can be deformed to make contact with the recessed sensor portion distal end when the probe 1400 is applied to the measurement site.

As shown in FIG. 14D, when a compressive force is applied to the distal end of the sensor portion 1410, such as when the probe 1400 is applied and/or attached to a measurement site, the spring 1450 can be compressed to retract the sensor portion 1410 proximally. The compressed spring 1450 can exert a distal force on the sensor portion 1410 to force the sensor portion 1410 into contact with the measurement site. The distally directed force of the compressed spring 1450 can improve contact between the sensor portion 1410 and the measurement site. The compressed spring 1450 can provide a predetermined pressure at the skin-sensor interface to as to improve optical and acoustic coupling between the sensor and the skin.

### Example Applications of Malaria Probe Embodiments

Embodiments of the malaria probe disclosed herein can be used in various applications to diagnose malaria. For example, a malaria probe, such as one having the sensor portion 510 of FIG. 5 or any other embodiments of a malaria probe described herein, can be applied to a patient's skin at various parts of the patient. Suitable locations on the patient for a transdermal application can include the digits, hand, wrist, ankle, neck, earlobes, lips, tongue base, or others.

As shown in FIGS. 8A-8C, malaria parasites 804 can escape from the blood 802 to the tissue and/or skin 800 in a process called sequestration. While antimalarial drugs may be effective against the malaria parasites 804 in the blood 802, the drugs may not be effective against the malaria parasites 804 that hide in the tissue 800. As shown in FIG. 8C, some parasites 804 can survive in the tissue and/or skin 800 after the parasites 804 in the blood 802 have been killed. Whereas the parasites that circulated in the blood stream can usually be in young and unpigmented (ring) forms, the parasites that accumulated in the small blood vessels (sequestered) were mature, with the elevated level of hemozoin. The accumulation of hemozoin (in parasites and parasite-free tissue) in subcutaneous layers of skin within the depth of about 100-500 um may include the following hypothetical mechanism:
Sequestration of mature parasites (including gematocytes) and/or hemozoin from peripheral blood into sub-cutaneous microvasculature. This may be typical for late-stage parasites with the highest level of hemozoin. Due to adhesion mechanisms, such parasites attach to wals of subcutaneous micro-vessels and, eventually, becomes immobilized in upper skin layers that contain such micro-vessels. Thus hemozoin accumulates in skin more than in blood.
In skin, the sequestered parasites and hemozoin can persist for months while they clear from peripheral blood in days.
Sequestered parasites (including gematocytes) are responsible for lethal complications in the clinic, uncontrollable transmission, and relapse, and represent a hidden pool of malaria.

The transdermal (skin) application can be effective not only in detecting and/or killing malaria parasites in the patient's blood. The malaria probe disclosed herein can have sensitivity and/or specificity to detect tissue-sequestered malaria parasites and/or hemozoin nanoparticles by generating nanobubbles around the malaria-specific nanoparticles in the micro-capillaries in the tissue.

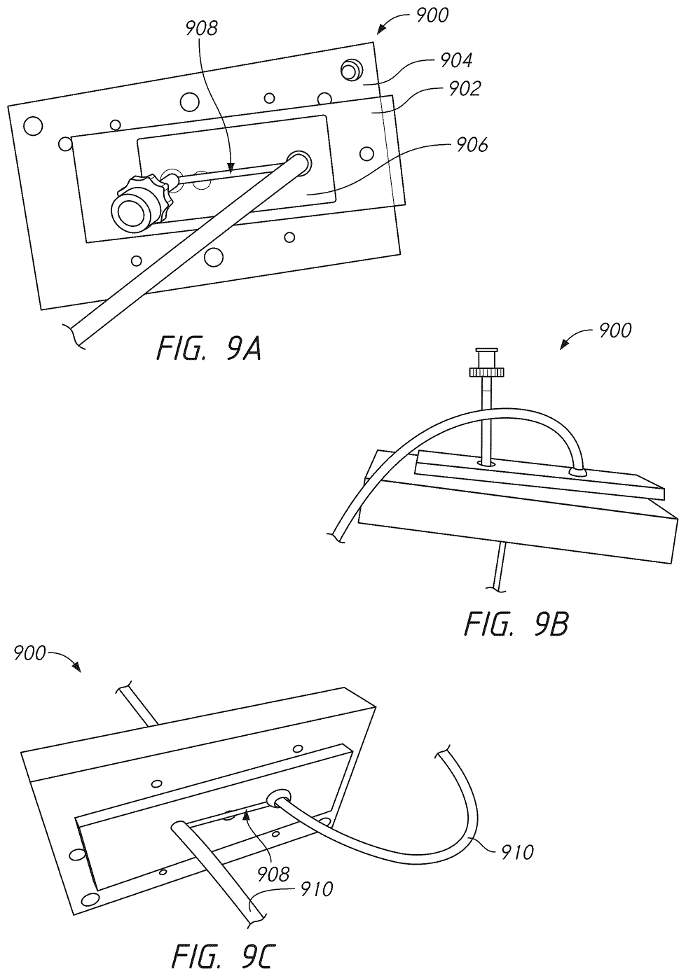

The malaria probe disclosed herein not only has in vivo applications, but can also be used to detect malaria parasites ex vivo, to analyze blood, urine or other body fluids. FIGS. 9A-9C illustrate an example flow cuvette 900 that can be used with a malaria probe. The flow cuvette 900 can include a glass-silicon cuvette 902 sandwiched between a metal base 904 and a glass cover 906. The cuvette 902 can contain a flow path 908. The flow path 908 can be coupled to a tube 910 at each end of the flow path 908. In some embodiments, the malaria probe can direct one or more laser pulses to a patient's blood or urine sample flowing through the flow path 908 to detect malaria parasites in the sample, such as by using the process 200 shown in FIG. 2. The blood or urine sample can have a flow rate of about 5 mm/s. The flow rate can be controlled by a syringe pump or any other pumps.

The application of the probe on the liquid sample (for example, such as the peripheral blood) and on the skin stem from different biological mechanisms: active disease (peripheral blood) vs transmission (skin). Hence the peripheral blood cannot be analyzed “through the skin” and parasites or hemozoin properties do not correlate in peripheral blood and skin. Also, these applications have opposite scientific back-ups: well-established for blood and thus may provide an additional test if conventional blood test described above for malaria turns out to be inconclusive, and almost non-existent science for malaria in skin.

It is the skin rather than the blood that may be more challenging in the eradication of malaria. Malaria transmission can start with mosquitoes picking up parasites from the skin of an infected patient, specifically, the gametocytes from the subcutaneous layers of skin of the infected patient. Among all blood stages of parasites, these are the gametocytes that can deliver the maximal nanobubble signal because they have the highest quantity and size of hemozoin particles. The gametocytes, after becoming biologically inert, may last for many months in the skin. A transmission of malaria requires gametocytes be available for mosquito bites, that is, in the subcutaneous upper skin layer, which can be less than 1 mm in thickness. This biological mechanism delivers hemozoin to where it can meet the laser pulses emitted from the malaria probe examples disclosed herein (for example, at about 0.1 mm to about 0.5 mm optical penetration depth). The higher level of hemozoin in gametocytes, compared to other stages of parasites, can result in the higher nanobubble signal as found both in the laboratory and human studies disclosed herein.

The systems for noninvasively diagnosing and/or treating malaria can have multiple applications. The systems can have a diagnostic application for determining if a patient carries malaria-specific nanoparticles, hemozoin. If nanobubble(s) generation is detected using the processes, parameters and thresholds described herein, the patient can be diagnosed as malaria positive, including the residual malaria without active disease symptoms. If the nanobubble(s) generation is not detected, the patient can be diagnosed as malaria negative. The malaria-positive patients may have been treated when the nanobubble generations have caused mechanical destruction of the malaria parasites. The malaria-positive patients may additionally or alternatively be treated with anti-malarial drugs.

The systems can also have an epidemiological application for determining a malaria-infected region and/or the foci of malaria transmission. Caregivers typically provide anti-malarial drugs to a large population when there is an outbreak of malaria in a region. Regulators such as the WHO also issue rules and reports on the administration of anti-malaria drugs to various populations. As current malaria detection methods have difficulty detecting asymptomatic, sub-potent, past malaria infections, especially when peripheral blood is malaria-free, and are thus limited in locating where malaria originated, providing treatments to a large population, such as across an entire country, may be the only way to prevent transmission of malaria within that population. Anti-malarial drugs can have undesirable side effects, such as causing miscarriage in pregnant women, heart failures and other dangerous conditions. It is therefore desirable to narrow down the malaria-infected region/populations as much as possible to avoid administering anti-malarial drugs to people who may not need the drugs and/or may be harmed by the drugs.

The systems and processes described herein provide ways to narrow down the malaria-infected region. The human body carries malaria-specific nanoparticles, such as the HZ (hemozoin) nanocrystals, so long as the body was once a host of active malaria parasites. Even when the body no longer carries active malaria parasites, for example, if the malaria parasites went dormant and/or if the body had been infected by malaria parasites in the past, the HZ (hemozoin) nanocrystals can still be present in the body, especially in the skin. The ability of the system to detect the HZ (hemozoin) nanocrystals with or without an active malaria parasite using laser-induced nanobubbles can allow detection and/or narrowing down of geographical regions and population where malaria is endemic, and/or where malaria originated. Caregivers can treat a smaller population in the narrowed-down region, such as a small village, with anti-malarial drugs or other malaria treatments in order to contain the spread of malaria. The more localized and/or targeted administration of anti-malarial drugs can be effective in malaria eradication without unnecessarily dosing a large population with the drugs.

For geographical mapping of the malaria transmission through the mass screening of the population, the confirmed geographical location of human subjects can be mapped in a way such that the mapping shows the relative level of malaria positive (hemozoin-positive) signals for specific areas where the screening (collection of the signals) is performed. This can be achieved by calculating the relative level of malaria- or hemozoin-positive subject normalized by the total number of screened subject per specific area (for example, a village, a border checkpoint, a clinic, or other specific geographical location). Such an approach would be more efficient for a settled “stationary” population. The mapping of dynamic “transit” population (such as at a border checkpoint or an airport) would require additional information on the geographic origin of human subject. In either case, the absolute number of positive detections can be considered also after being normalized by the total number of subjects screened from the area in question.

FIGS. 33A and 33B illustrate examples of tracking malaria and/or its transmission. As shown in FIG. 33A, at step 3302, a controller of a malaria detection system can detect a GPS location of the malaria probe. The controller can apply laser pulses to a human subject via the probe at step 3304. At decision step 3306, the controller can analyze acoustic signals received from the probe to determine whether the subject is malaria positive. If malaria parasites or HZ (hemozoin) crystals are detected in the subject, at step 3308, the controller can output an indication of malaria detection. If malaria parasites or HZ (hemozoin) crystals are not detected in the subject, at step 3310, the controller can output an indication of no malaria detection. At step 3312, the controller can upload the outcome of the malaria detection procedure and the location of the probe to a server, such as a cloud server. In some embodiments, the GPS may only be activated upon having determined that the subject is malaria positive by the sensor, and/or for a subject that is local (as opposed to being transient) the location to be tracked by the GPS.

The mapping of the malaria transmission may additionally involve the analysis of local demographics so the signals are analyzed not just as a function of the coordinates of the screened area but also of the demographic, climate and medical parameters of the screened area (age, gender, ethnicity, income, presence of other diseases, time of the year, stage of malaria transmission season, etc.) and other factors which are related to the transmission of malaria. Such analysis can be performed locally or remotely by uploading the signals and other area-specific data to the remote server. Such analysis would result in an epidemiological “maps” of malaria transmission which may be very useful for the treatment, elimination and prevention of malaria. As shown in FIG. 33B, the server can receive GPS locations from a plurality of malaria probes at step 3320. At step 3322, the server can receive malaria detection information of one or more human subjects at the plurality of locations from the probes. At step 3324, the server can also optionally receive demographic data at the plurality of locations (for example, via government databases). At step 3326, a processor at the server can plot a malaria transmission data at least in part based on the information received from the malaria probes.

Collection and analysis of malaria population data will now be described in more detail. It is often reported that there is insufficient information with which to deploy the available resources for controlling the transmission malaria. Many malaria-endemic countries still suffer from weak health-management information systems and often lack vital registration. At a global level, only around 10% of estimated malaria cases are detected. Many patients with suspected infections receive empiric antimicrobial therapy rather than appropriate therapy dictated by the rapid identification of the infectious agent. The result is overuse of a small inventory of effective antimicrobials, whose numbers continue to dwindle due to increasing levels of antimicrobial resistance. As shown in FIG. 33C, a device and data acquisition global system can solve both of these challenges to malaria elimination in less developed nations. Due to its digital signal capture, it has the ability to change the paradigm for screening, case identification and population monitoring in both endemic and malaria-free countries. The system can establish a global “nanobubble platform” for real-time epidemiologic data capture and analysis to guide a more efficient allocation of scarce health resources, and to monitor the residual malaria in treated patients to determine therapeutic (including experimental vaccines) efficacy.

In geo-tagging of malaria, primary data can be further combined with fingerprinting device incorporated with the malaria sensors for reliable identification of all screened subjects. With a properly designed and connected global data base, such information may be used by border and passport control authorities to track the malaria carriers and thus to contain the malaria infection and prevent its spread.

The table below describes the clinical and mass-screening applications of the malaria diagnostics disclosed herein.

| Technology | Non-invasive mass screening | Clinical analysis of blood |
|---|---|---|
| Application | Epidemiological: Monitoring and | Clinical: Confirmation of the malaria |
| | screening of the malaria | diagnosis (positive or negative) in |
| | transmission | clinic for patients with unclear RDT |
| | | and microscopy data |
| Method and | Non-invasive detection of skin | Express analysis of blood sample, |
| samples | signals for parasites and | including venous blood (currently |
| | hemozoin in skin | used for high-sensitivity PCR) |
| Hemozoin | Estimate: 1-10 | 0.1-0.5 |
| detection | | |
| threshold, | | |
| analog of par/uL | | |
| Potential Users | Health workers, boarder and | Clinics, hospitals, research labs and |
| | epidemiological administration, | manufacturers of malaria drugs and |
| | governments and NGO | vaccines |
| Design | Portable, simple and rugged in | Rugged, to be used by microscope and |
| requirements | use by non-medical staff, | PCR (medical) users, can use external |
| | inexpensive, internal power | power |
| Market status | No current analogs, will solve a | Will improve the clinical use of RDT |
| | problem of monitoring malaria, | and microscopy where these methods |
| | transmission, which currently has | are inconclusive and ultra-sensitive |
| | no solution in global scale | PCR is not available |

### Example Methods of Malaria Detection Using Peak Time-Delay

As described above, in addition and/or alternative to monitoring the parameters derived from the amplitude of acoustic signal when one or more predetermined laser pulses are applied to a measurement site, the systems described herein can independently detect nanobubbles generated around malaria-specific nanoparticles in a tissue based at least in part on a signal peak time-delay. The signal peak time-delay can be used as an additional diagnostic metric, which is independent of the metrics derived from the signal amplitude, such as the normalized positive count, N, and the hemozoin index, HI described above. The hemozoin index can represent a relative signal amplitude above an amplitude threshold. The normalized positive count can represent relative number of signals above the amplitude threshold. The peak timing metric can be used together with the signal amplitude metrics (N and HI) to increase the diagnostic sensitivity and specificity of the clinical diagnostics and/or mass screening.

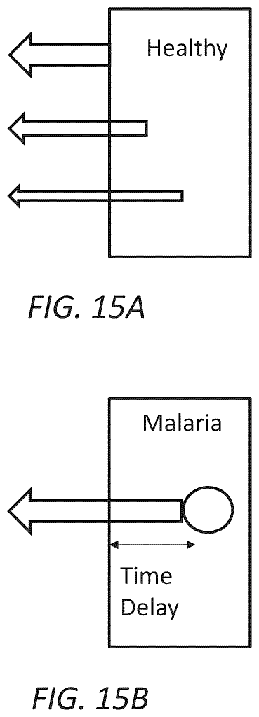

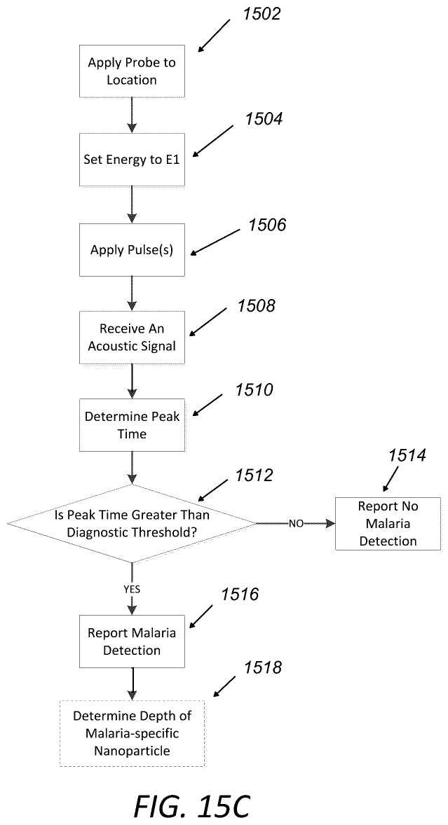

FIGS. 15A and 15B illustrate schematically acoustic pulses from healthy tissues and from malaria-infected (or malaria-positive) tissues when one or more predetermined laser pulses are applied to the tissues. As shown in FIG. 15A, heat generated by the bulk residual optical absorption of the laser pulse by components of healthy tissue (proteins, melanin, blood components) can produce a thermos-elastic stress in the patient's skin regardless of whether the patient is infected with malaria. This thermos-elastic stress results in a background acoustic signal. In malaria-negative (healthy) tissues, the acoustic pulses are the strongest at the skin surface. This is because the source of the acoustic pulses is the skin that absorbs the heat from the laser pulse(s), with the maximal optical fluence being at the skin surface. The deeper skin attenuates the light exponentially with the depth due to optical scattering and absorption. Due to the light attenuation in malaria-negative tissues, the thermo-elastic background signal decreases with the depth from the skin surface.

When the tissue is infected with malaria, the source of the acoustic pulses also includes nanobubbles generated around malaria-specific nanoparticles. The acoustic signal from nanobubbles can be different from the background acoustic signal from the skin. The acoustic signals from nanobubbles can be delayed by the time determined by the depth of nanobubble and the speed of sound.

Tissues with subcutaneous malaria-specific nanoparticles result in the maximal nanobubble-emitted pressure pulse, which is at some depth from the skin surface. The distance traveled by the pressure pulse to the sensor and from subcutaneous parasites, including the malaria-specific nanoparticles, can be higher (such as slightly higher) than the distance a thermos-elastic wave travels from the skin surface. The distance traveled by the pressure pulse from nanobubbles can be determined by the depth of the malaria-specific nanoparticles, such as HZ (hemozoin) nanocrystals. The malaria-specific nanoparticles can be located at the depth from about 10 μm to about 400 μm, or to about 500 μm below the skin surface. About 500 μm below the skin surface is not the location limit of the malaria-specific nanoparticles, but can be the maximal depth of the optical penetration of the laser pulse which still can generate a nanobubble around malaria-specific nanoparticles. Due to the additional travel distance, the acoustic pulses from the nanobubble generation also arrive at the sensor probe with a time delay compared to the background acoustic pulses from the skin surface. The time-delay due to the additional travel by the acoustic pulses can be determined by the speed of sound in tissue and the depth of HZ (HEMOZOIN) location. The time-delay can be about from 40 ns to about 300 ns, or to about 333 ns (assuming that sound travels at 1600-1800 m/s in the tissues).

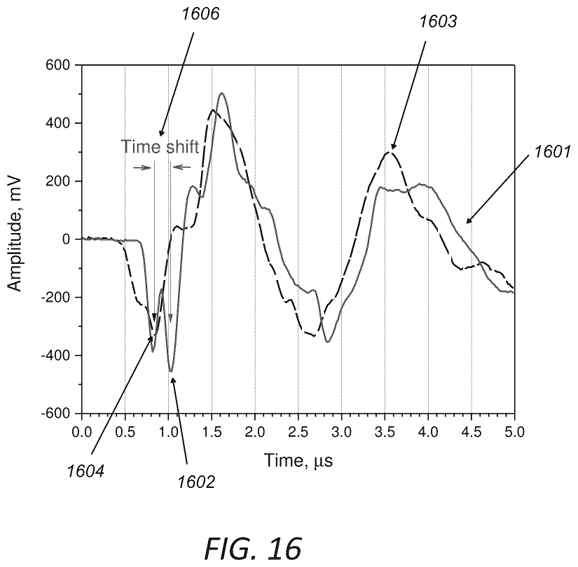

When comparing the acoustic signals from malaria-negative (healthy) tissues and malaria-positive tissues, such as shown in FIG. 16, there can be a time-shift 1606 to the right of the highest signal peak 1602 in the malaria-positive signal 1601 (solid line) compared to the highest signal peak 1604 in the malaria-negative signal 1603 (dashed line). The signal peaks 1602, 1604 can have a negative value due to the negative charges formed upon the acoustic pulse reaching the piezo element.

The depth of the malaria-specific nanoparticle can be calculated by multiplying the speed of sound in the tissue and the time delay. As shown in FIG. 16, the malaria-positive signal has a time-delay 1606 of about 120 ns. The malaria-specific nanoparticle can thus be located about 0.3 mm deeper than the skin surface (in the illustrated example).

The time-delay in the malaria-positive signal and the background signal can be detected with an ultrasound detector with sufficient temporal resolution, such as the acoustic detector described herein. The ultrasonic detector can have a frequency of at least about 4 MHZ (hemozoin) or more to detect a signal peak time from the malaria-positive and malaria-negative signals.

A peak timing-delay diagnostic threshold can be used as an additional and/or independent malaria diagnostic criterion. Signals with the peak timing below the diagnostic threshold can be assumed to indicate a malaria-negative status, and signals with the peak timing above the diagnostic threshold can be assumed to indicate a malaria-positive status. The diagnostic threshold can be predetermined, for example, using empirical data. As will be described below, the threshold can be determined based on the peak timing-derived time histograms.

FIG. 15C illustrates an example process for detecting malaria based at least in part on the peak time-derived diagnostic threshold. At step 1502, one or more signal processors of a malaria diagnosis system can instruct a user, such as a clinician, to apply the malaria probe, such as any probe disclosed herein, to a target location. The target location can be any of the test subject described above, including but not limited to the ankle, base of the tongue, hand, described herein. The step 1502 can include applying a layer of gel to a probe tip surface before applying the probe to the target location. The layer of gel can improve acoustic and/or optical coupling between the malaria probe and the patient's skin. At step 1504, the one or more signal processors can set an energy level of a laser pulse generator to a first predetermined level, E1. E1 can be sufficient for generating nanobubbles around malaria-specific nanoparticles up to about 0.5 mm underneath a surface of the target location. At step 1506, the one or more signal processors can cause the laser pulse generator to apply one or more laser pulses having an energy level of E1 to the target location. As described above, the one or more laser pulses can have the same or different energy levels and/or wavelengths.

At step 1508, the one or more signal processors can receive an acoustic signal from the acoustic detector of the probe. The signal can be indicative of the background acoustic pulses generated by the skin, and/or the acoustic pulses from the generation of nanobubbles (if malaria-specific nanoparticles are present at the target location). At step 1510, the one or more signal processors can determine a peak time of the signal. At decision block 1512, the one or more signal processors can determine whether the peak time of the signal exceeds a predetermined diagnostic threshold. The diagnostic threshold can vary depending on the type of sensor, the type of target location or measurement site, the depth of the malaria-specific nanoparticles, and/or the species of the malaria parasite.

If the peak time does not exceed the diagnostic threshold, the one or more signal processors can output a message that no malaria is detected at step 1514. If the peak time exceeds the diagnostic threshold, the one or more signal processors can output a message that malaria is detected. At decision block 1512, the one or more signal processors can also combine the determination based on the peak time with the signal amplitude parameters, such as the N and HI values. For example, if the signal peak time does not exceed the diagnostic threshold, but the signal amplitude parameters are highly indicative of a malaria-positive status, the one or more signal processors can output a malaria-positive status message. At the decision block 1512, the one or more signal processors can also compare the signal amplitude parameters with a signal amplitude parameter threshold instead of comparing the peak time of the signal with the peak time threshold.

At step 1518, the one or more signal processors can also optionally determine the depth of the malaria-specific nanoparticle based at least in part on the peak time of the malaria-positive signal.

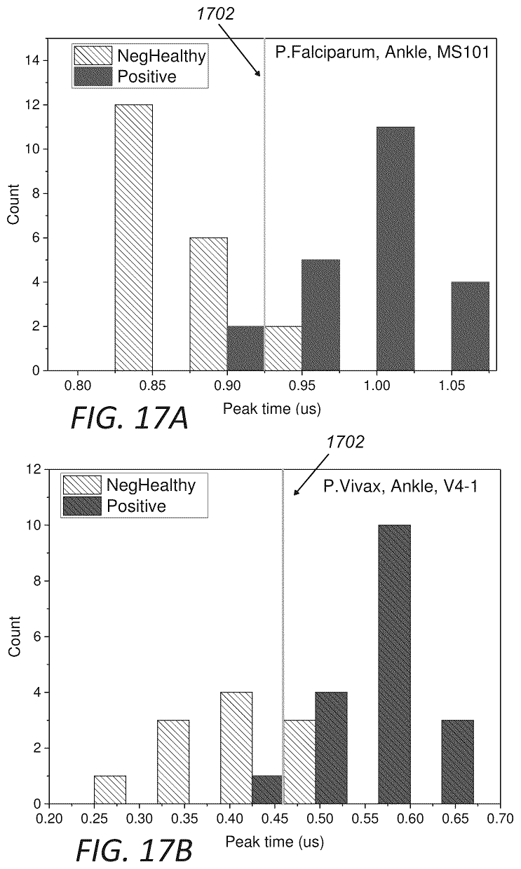

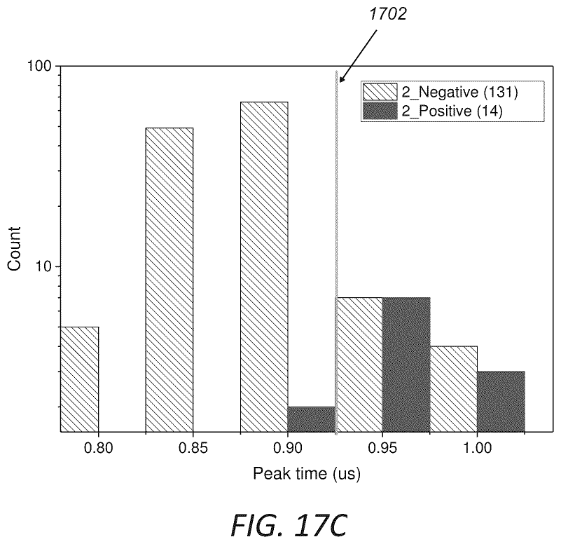

FIG. 17A illustrates a peak timing-derived time histogram of an example application of the time-delay analysis on 22 malaria-positive subjects and 20 malaria-negative subjects in non-invasive human study with the blinded data collection. The study was targeted at the malaria parasite P. Falciparum. Each subjects received 3 sets of 60 signals on the ankle. The diagnostic threshold 1702 is set to be 0.925 s. Most of the observed peak time-locations for the malaria-negative signals are below 0.925 s. Most of observed peak time-locations for the malaria-positive signals are above 0.925 s.

As shown in FIG. 17A, the shape of the peak timing-derived time histogram formalaria-negative signals matches the exponential nature of the signal attenuation with the tissue depth. In contrast, the peak timing-derived time histogram for malaria-positive signals shows an increase of the signal peak with the tissue depth, up to the penetration range of the laser beam.

Table 1 below illustrates an example two-sample t-test performed on the results of the human study of the application illustrated in FIG. 17A. This t-test finds two sub-populations, malaria-negative and positive (as independently was determined with the standard microscopy procedure) being significantly different

TABLE 1

| N | Mean, μs | SD, μs | SEM | Median, μs |
|---|---|---|---|---|
| Peak time, | 0.86881 | 0.04669 | 0.01019 | 0.87 |
| negative” | | | | |
| 21 | | | | |
| “Peak time, | 0.9825 | 0.04185 | 0.00892 | 0.995 |
| positive” | | | | |
| 22 | | | | |
| | | t Statistic | DF | Prob > \|t\| |

| | | | |
|---|---|---|---|
| Equal Variance Assumed | −8.4163 | 41 | 1.78932E−10 |
| Equal Variance NOT Assumed | −8.3945 | 40.0233 | 2.31542E−10 |
| (Welch Correction) | | | |

As shown in the table, there can be a 110/130 ns average/median delay in the timing of peaks of malaria-positive signals compared to malaria-negative signals. The mean time-delays can be statistically significant, regardless of whether equal variance is assumed.

FIG. 17B illustrates a peak time histogram for subjects suspected of infection by the malaria parasite P. Vivax. The malaria probe of another design was applied to the ankle than the malaria probe used in the test illustrated in FIG. 17A. The diagnostic threshold can be about 0.46 s for the parasite Vivax. Accordingly, the peak time-delay diagnostic threshold can vary based on the malaria parasite species and the sensor design.

The human subjects were also tested for malaria using standard microscopy and PCR tests of peripheral blood samples. As shown in FIGS. 17A and 17B, the diagnostic thresholds 1702 correspond substantially with the determination using microscopy and PCR test, with a sensitivity of 91% for P. Falciparum and a sensitivity of 95% for P. Vivax. For both malaria species, the difference in average time-delays (about 70 ns to about 120 ns) corresponds to about a 200 μm depth of the malaria parasite and/or the malaria-specific nanoparticles from the skin surface.

FIGS. 17A and 17B illustrate clinical applications of the malaria detection sensor probes disclosed herein. In a clinical application, the subjects visit a healthcare facility, for example, to seek medical assistance. FIG. 17C illustrates using the malaria-detection sensors disclosed herein for mass screening (which usually assumes an asymptomatic infection). Mass screening can be performed by healthcare professionals taking the malaria detection sensors to a group of people, for example, all residents of a remote village, and using the sensors on the entire group of people. Mass screening using the malaria detection sensors disclosed herein can allow detection and/or narrowing down of geographical regions where access to healthcare facilities and/or personnel is difficult or infeasible, malaria is endemic, and/or where malaria originated, as described above. Current malaria detection methods, such as described in the Background section of the present disclosure, may not be suitable for mass screening. Compared to the nanobubble-based malaria detection methods disclosed herein, those methods are more time-consuming and/or demanding on equipment that may not be designed to function in a rugged environment. Current methods rely on the presence of malaria in a peripheral blood, but parasites can often escape to tissues though the mechanism known as a sequestration.

FIG. 17C illustrates a peak timing-derived time histogram for a mass screening study on 145 subjects with the ankle as the measurement site. The diagnostic threshold 1702 was pre-set to be 0.925 s. The subjects were also tested for malaria using microscopy and PCR tests of the subjects' peripheral blood samples. Compared to the determinations using microscopy and PCR tests of peripheral blood samples, the peak time-based test had a sensitivity of about 86%.

As also shown in FIG. 17C, results of some malaria-negative subjects as determined from peripheral blood tests are above the peak time diagnostic threshold. This can be due to the tissue-sequestered hemozoin described above, leaving the peripheral blood of those subjects free of hemozoin. As discussed above, the nanobubble based malaria detection technology as disclosed herein can detect tissue-sequestered malaria infection when microscopy and PCR tests of peripheral blood samples indicate that the subject is malaria-negative.

In another example study of nanobubble based malaria detection, different types of sensor probes were used on the patient's ankle, back of hand, blood, and urine samples respectively and different diagnostic parameters were used. Data was collected from clinical studies using the malaria sensor described herein in various stages.

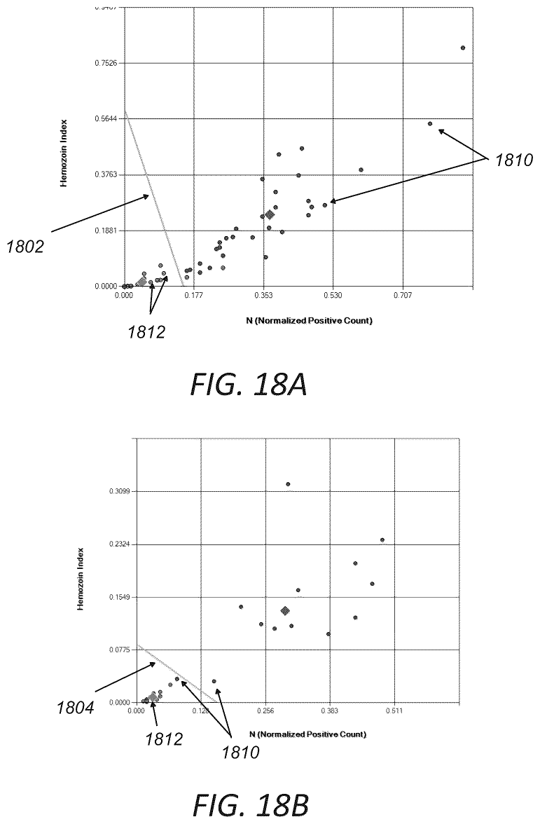

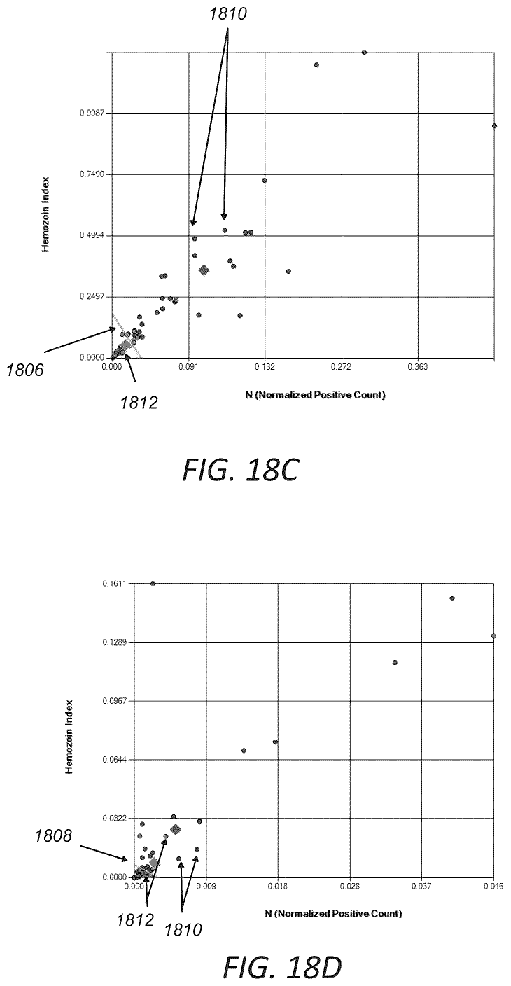

In addition to peak time as the diagnostic parameter, the signal amplitude-derived diagnostic parameters were used for some of the tests on the ankle. The subjects were tested for malaria using a signal amplitude-based diagnostic parameter (N-HI) for the rest of the tests. FIGS. 18A-18D illustrate the N-HI plots and the amplitude parameter-based diagnostic thresholds. FIG. 18A illustrates an N-HI diagnostic threshold 1802 when the test is performed on the ankle. FIG. 18B illustrates an N-HI diagnostic threshold 1804 when the test is performed on the back of the hand. FIG. 18C illustrates an N-HI diagnostic threshold 1806 when the test is performed on the blood sample. FIG. 18D illustrates an N-HI diagnostic threshold 1808 when the test is performed on the urine sample.

The subjects were also tested for malaria using current diagnostic tools such as the RDT, PCR and microscopy. In FIGS. 18A-18D, the malaria-positive subjects as determined by RDT, PCR and microscopy are shown as red (or darker) dots 1810. The malaria-negative subjects as determined by RDT and microscopy are shown as grey (or lighter) dots 1812. In case the RDT, PCR and microscopy data did not match, up to three microscopy reading were done independently and the microscopy data determined the malaria status.

Table 2 below summarizes the sensitivity and accuracy (as defined above) of the various tests in the example study.

TABLE 2

| | Ankle | | | Back of | Blood | | Enrollment |
|---|---|---|---|---|---|---|---|
| Location | Type | Type | Type | Hand | Type | Urine | (malaria/ |
| Sensor | 1 | 1 | 2 | Type 1 | 3 | Type 3 | healthy) |
| Diagnostic | Peak | N-HI | N-HI | N-HI | N-HI | N-HI | |
| parameters | time | | | | | | |
| Stage 1B: Blinded (all settings and thresholds | | | | | | | 24 (12/12) |
| were pre-set and fixed) | | | | | | | |
| Sensitivity | 0.86 | 1.0 | 1.0 | 0.86 | 1.0 | 0.71 | |
| Accuracy | 0.88 | 1.0 | 0.88 | 0.92 | 0.79 | 0.75 | |
| Stage 1: Total | | | | | | | 55 (30/25) |
| Sensitivity | 0.91* | 1.0 | 0.94 | 0.87 | 0.97 | 0.68 | |
| Accuracy | 0.91* | 0.96 | 0.87 | 0.85 | 0.76 | 0.84 | |

*based on the total count of 43 (22/21) because peak time was not collected for all subjects

### Additional Example Human Study Data

Using the sensor probe such as shown in FIGS. 3A-3D, a skin volume of a patient was probed noninvasively. The skin volume probed had a diameter of approximately 100 um and a depth of 400-500 um. 3 tests were performed at 3 seconds each, collecting a total of 180 signals (60 pulses per test). Test locations included the tongue base, inner lip, ear lobe, wrist, hand, and ankle. The sensor probe was placed at several close and random areas within one location, via ultrasound gel.

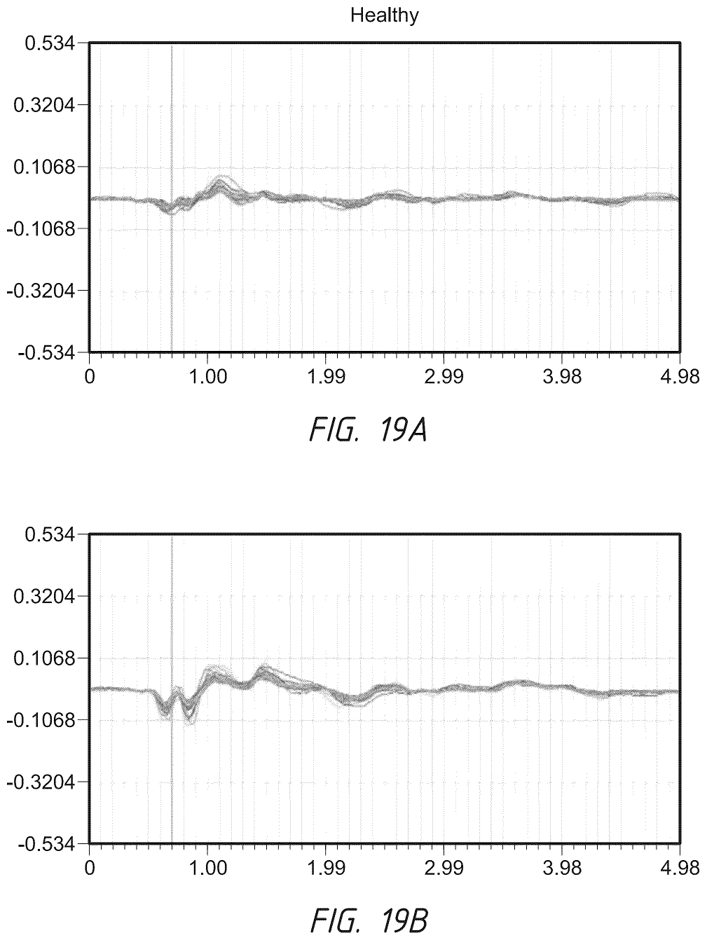

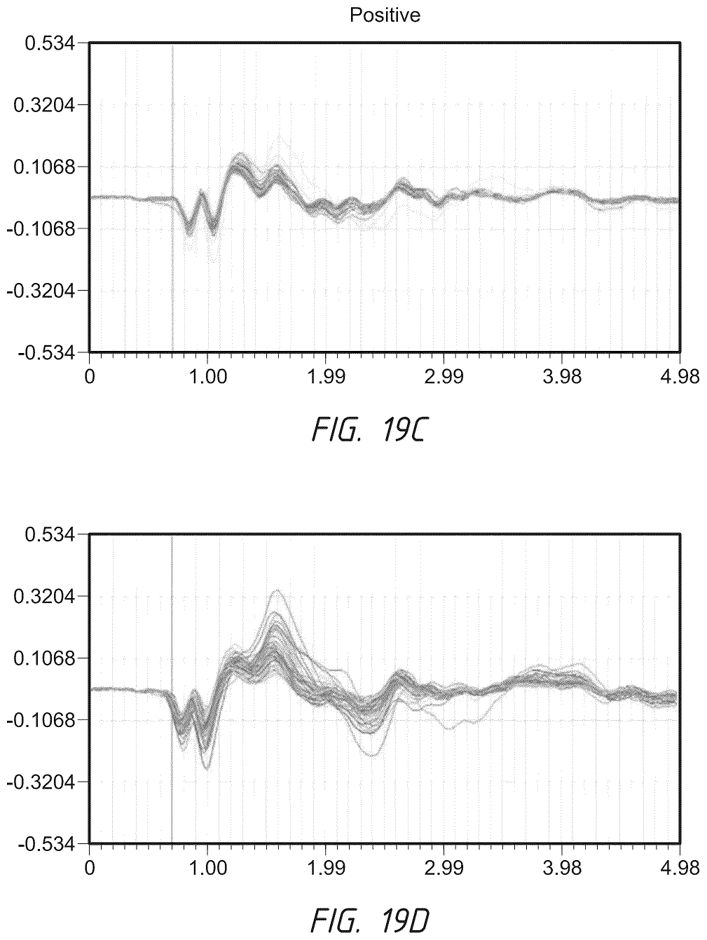

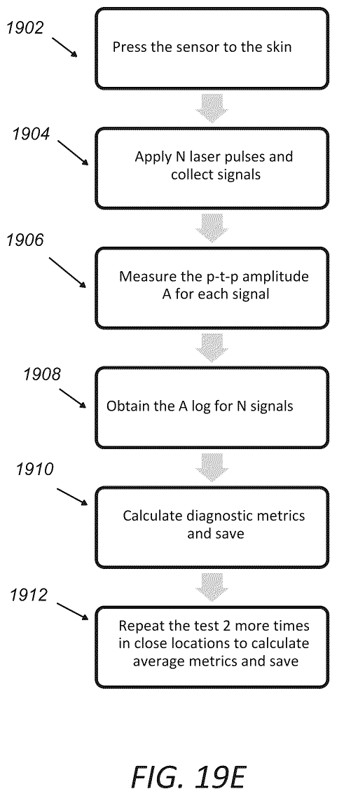

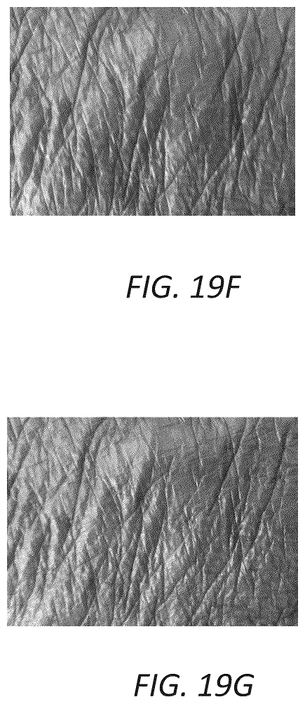

FIGS. 19A-19D illustrate two groups of 60 signals non-invasively collected from healthy (19A-19B) and positive (19C-19D) human subjects at the ankle. The healthy subjects did not have malaria within the past 1-3 years, were not from areas with high transmission, and were tested negative with microscopy. The negative subjects were from areas with high transmission, tested negative with microscopy, and often with confirmed history of malaria. Positive subjects were from areas with high transmission and tested positive with the standard malaria microscopy test.

FIG. 19E illustrates the diagnostic procedure performed. At step 1902, the malaria probe can be gently pressed to the skin. At step 1904, a plurality of, or N, laser pulses (for example, 60) can be applied while the user holds the malaria probe still for about 3 seconds. Signals were collected and inputted into a processor. At step 1906, the processor can measure the peak-to-peak amplitude A for each signal. At step 1908, the processor can obtain an A log for the N signals. At step 1910, the processor can calculate the diagnostic metrics disclosed herein and save the metrics, for example, to a memory device. At step 1912, the procedure can be repeated 2 more times in the same test location near the previous test site. The processor can calculate and save an average of the diagnostic metrics.

In addition to the skin tests, blood and urine tests were performed. 3 flow tests were performed with 1000 signals each. Capillary (peripheral) blood was taken with a finger prick. Reference methods, microscopy using the peripheral blood, RDT, and PCR, were also performed using peripheral (blood) samples.

The signal analysis can use the following equations for calculating the signal amplitude metrics in the liquid (urine and blood) sample and non-invasive skin tests. The normalized positive count Nₙₒᵣₘ can be calculated as

$${N}_{norm} = \frac{{N}_{pos}}{Ntotal} ,$$

where Nₚₒₛ is the number of signals above a threshold, and Nₜₒₜₐₗ is the actual number of laser pulses. The normalized amplitude above the amplitude threshold, HI, can be calculated as

$$HI = \frac{Σ ( A - T )}{Npos * T} {N}_{norm} ,$$

where T is the threshold signal amplitude and A is the peak-to-peak amplitude for the signals equal to or above T.

This diagnostic procedure was found to be safe and without any detectable damage to human skin. FIGS. 19F and 19G are images of human skin before and after the application of diagnostic laser pulses. As shown, no harm and no noticeable changes were can be observed on laser-probed skin, which was exposed to 180 pulses at 15 uJ per pulse. No discomfort or delayed complaints were observed in in more than 400 people that received the diagnostic procedure.

Table 3 below summarizes the sensitivity and accuracy (as defined above) of the various test in the example study. As shown, the worst signals (lowest amplitude, minimal separation of negative and positive components) were observed in the studies for an inner lip and a tongue base.

TABLE 3

| | | | | | Tongue |
|---|---|---|---|---|---|
| Location | Ankle | Hand | Wrist | Lip | Base |
| Sensitivity | 1.0 | 0.87 | 0.83 | 0.80 | 0.87 |
| Specificity | 0.92 | 0.84 | 0.84 | 0.87 | 0.73 |
| Separation of positive and | High | High | Medium | Low | Low |
| negative metrics HI and N | | | | | |

Data was collected in two studies from subjects in Sumatra and The Gambia. In Sumatra, the collected data was related primarily to the malaria species P. Vivax. In The Gambia, the collected data was related primarily to the malaria species P. Falciparum. Each study included two stages: a clinical application stage (Stage 1) and a mass screening stage (Stage 2). The clinical stage was further divided into Stage 1A and 1B. Additional details of the studies are provided in Table 4 below.

TABLE 4

| | Aim | | |
|---|---|---|---|
| | Determine the | | |
| | nanobubble-based | | |
| | method, device | Validate under the | |
| | settings and | fixed settings and | Screen endemic area, |
| | diagnostic | thresholds in blinded | blinded data collection |
| | thresholds (Stage | data collection (Stage | with previous settings |
| | 1A) | 1B) | and thresholds, Stage 2 |
| Enrollment | 31 | 24 | 145 |
| Standard | Peripheral blood | Peripheral blood | Peripheral blood |
| methods | | | |
| (Microscopy, | | | |
| RDT, PCR) also | | | |
| performed on: | | | |

| | | |
|---|---|---|
| Positive subjects | Peripheral blood is positive as confirmed with | Peripheral blood is |
| | standard methods, clinical symptoms may be | positive as confirmed |
| | present | with standard methods, |
| | | no clinical symptoms |
| Negative | Subjects did not have malaria in the past, | Peripheral blood is |
| subjects | peripheral blood is negative | negative, subjects often |
| | | had recent malaria |

As determined in the validation stage (Stage 1B, blinded) and shown in Table 5 below, when the malaria status of the blood and skin correlate, such as when malaria parasite and/or hemozoin are present in both the blood and in the skin, the sensitivity and specificity of the method of malaria detection using the sensors disclosed herein can be high. Throughout this disclosure, “healthy” and “negative” both denote subjects without malaria infection.

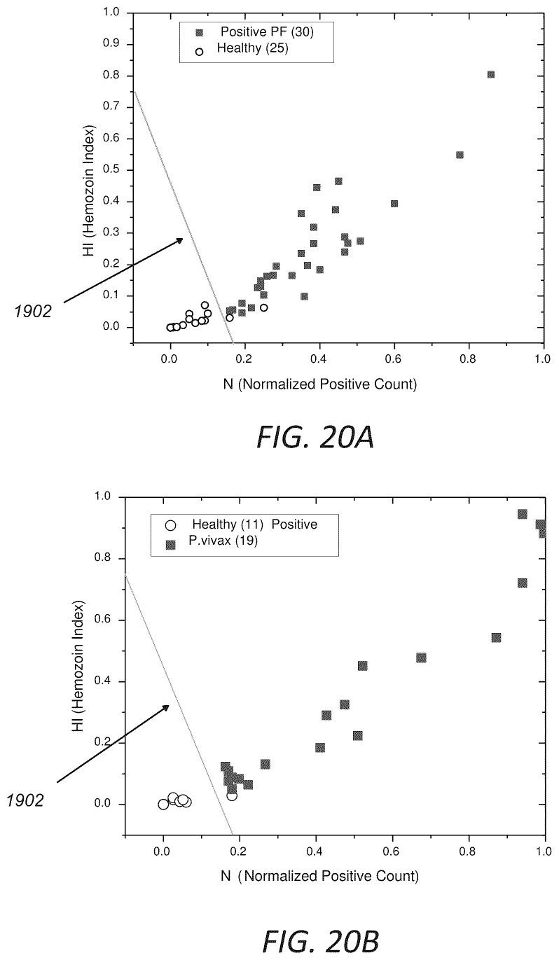

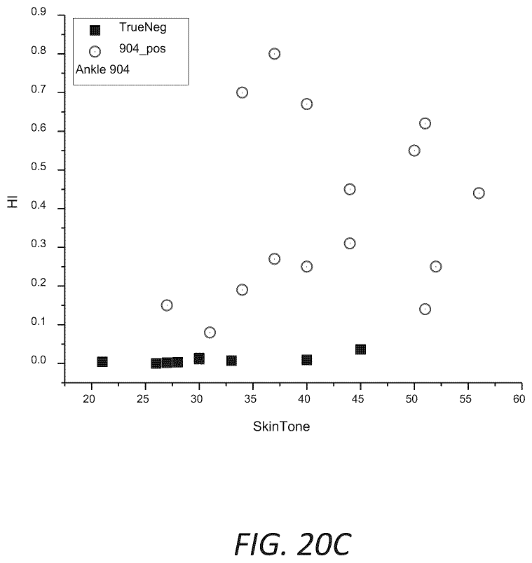

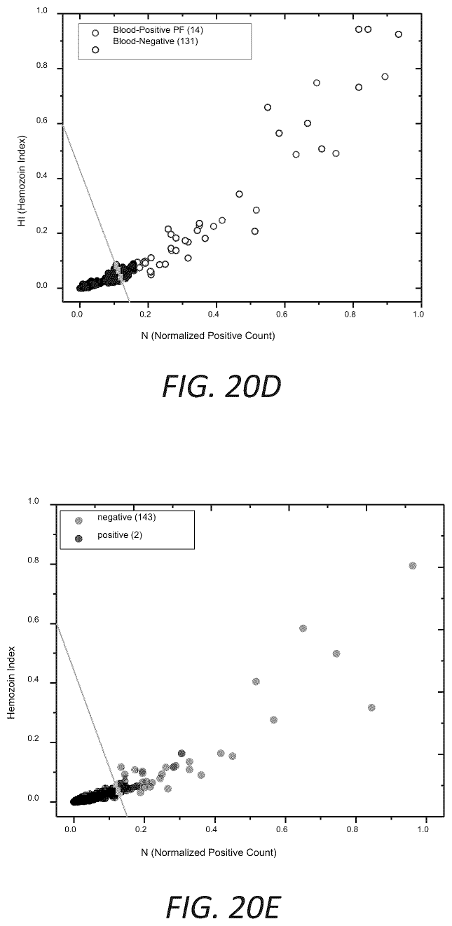

TABLE 5

| | P. Falciparum | |
|---|---|---|
| Best location: ankle | (The Gambia) | P. Vivax (Sumatra) |
| Validation Stage1B (blinded) | 14/10 | 10/14 (positive/negative) |
| | (positive/healthy) | (PA904) |
| | (MS101) | |
| Sensitivity (blood-based) | 100% | 90% (original threshold) |
| Specificity (blood-based) | 100% | 93% (original threshold) |
| Training + Validation stage | 30/25 (Type 1) | 19/11-The Gambia |
| data, positive/healthy | | threshold (Type 2) |
| (sensor type) | | |
| Sensitivity (blood-based) | 100% | 100% (The Gambia |
| | | threshold) |
| Specificity (blood-based) | 92% | 91% (The Gambia |
| | | threshold) |
| HI-N diagnostic metric | See FIG. 20A | See FIG. 20B |
| diagrams for Stage 1 total | | |
| Line 1902-pre-determined | | |
| diagnostic thresholds | | |

For both types of malaria, in the above shown two independent blinded studies, a good separation of data for positive and healthy subjects for suspected clinical cases were found. As shown in FIG. 20C, the skin tone did not influence malaria metrics. A skin tone was measured with the skin tone device in Sumatra (medium tone skin). In The Gambia study of Plasmodium Falciparum malaria, the skin tone device returned saturated signals as all skin was too dark for the skin tone device.

As determined in the mas screening stage (Stage 2) and shown in Table 6 below, when the malaria status of the skin and the blood differ, such as when there is no malaria parasite and/or hemozoin in the blood but sequestered malaria parasite and/or hemozoin in the skin, the method of malaria detection using the sensors disclosed herein cannot be benchmarked against the standard methods on peripheral blood. Another skin-based reference method is required to validate the method using the sensors disclosed herein.

TABLE 6

| | P. Falciparum | |
|---|---|---|
| Metrics used: HI-N | (The Gambia) | P. Vivax (Sumatra) |
| % of hemozoin-positive | 34.4% (50 cases | 9.7% (14 out of 145)- |
| as detected with our | out of 145) | original threshold |
| method | | 28% (41 out of 145) The |
| | | Gambia threshold |
| % of parasite-positive in | 9.7% (14 cases | 1.4 (2 out of 145) |
| peripheral blood | out of 145) | |
| Sensitivity against the | 86%* 100%** | 100% with threshold as |
| peripheral blood methods | | in The Gambia |
| Specificity against the | 69%* 65%* | 73% with threshold as in |
| peripheral blood methods | | The Gambia |
| % of hemozoin-positive as | 34.4% (50 cases | 9.7% (14 out of 145)- |
| detected with our method | out of 145) | original threshold |
| | | 28% (41 out of 145) The |
| | | Gambia threshold |
| HI-N diagnostic metric | See FIG. 20C | See FIG. 20D |
| diagrams for Stage 1 total | | |
| Line 2002-pre-determined | | |
| diagnostic thresholds | | |
| **For ankle + hand as | | |
| measurement | | |
| sites, 5 metrics | | |

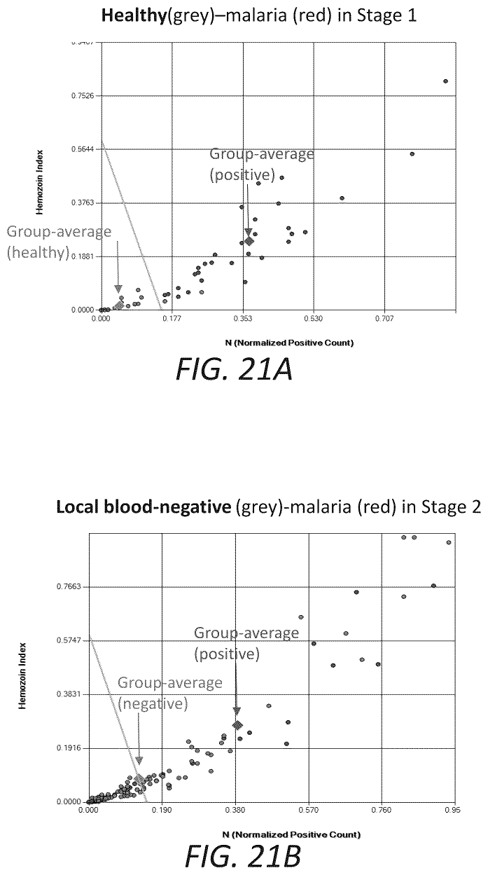

In the study of both clinical and asymptomatic cases, the malaria status was also determined through the microscopy and PCR analyses of peripheral blood samples. For clinical cases that are associated with the acute disease, which in turn develops in the peripheral blood, there was a good correlation between the blood malaria status and hemozoin-generated vapor nanobubble data as found both for Plasmodium Vivax and Plasmodium Falciparum types of malaria parasites. As determined by comparing the data from The Gambia in the clinical application stage (Stage 1, see FIG. 21A) and the mass screening stage (Stage 2, see FIG. 21B), the signals of these two subject groups with malaria infection in a highly endemic area were similar. The situation, however, has significantly changed for asymptomatic cases: a high level of blood-negative local subjects yielded hemozoin-generated vapor nanobubble-positive signals. As indicated by the negative group-average arrows in FIGS. 21A and 21B, the signals of healthy subjects differed from the blood-negative local subjects in a highly endemic area. According to the studies described herein, many local blood-negative subjects were not healthy subjects, they might have unknown malaria history and resided in highly endemic area. Their difference from healthy subjects can be seen from the comparison of the hemozoin-generated vapor nanobubble data. Table 7 below lists the ratios for average Stage 2 metric values to average Stage 1 metric values in the data from The Gambia, where ankle was the testing site.

TABLE 7

| | N | N | HI | HI |
|---|---|---|---|---|
| | positive/ | negative/ | positive/ | negative/ |
| Diagnostic metric | positive | healthy | positive | healthy |
| Ratio for average value | 1.1 | 2.9 | 1.3 | 5.6 |
| Stage2/Stage 1 | | | | |
| (ankle) | | | | |

As described above, the biological mechanisms causing the difference in the group average values could be at least the sequestration of parasites and hemozoin from the peripheral blood into subcutaneous microvasculature. As shown in Table 8 below, it can be typical for patients having late-stage malaria parasite infections, which would have produced a high level of hemozoin, to have more hemozoin accumulated in the skin than in the blood, or even to have malaria-free peripheral blood but still have parasites and/or hemozoin in their skin. In skin, the sequestered parasites and/or hemozoin can persist for months, whereas the parasites and/or hemozoin can clear from the peripheral blood in days. Sequestered parasites and/or hemozoin can be responsible for lethal complications in the clinic, such as uncontrollable transmission and relapses. Sequestered parasites and/or hemozoin can represent a hidden source of malaria injections. As described above, no standard method based on blood testing can detect the tissue-sequestered malaria parasites and/or hemozoin. Skin can also be a better source for malaria screening than standard methods using peripheral blood.

TABLE 8

| | | Latent, |
|---|---|---|
| | | sub-potent or |
| | | asymptomatic |
| Disease stage (duration) | Acute (days) | (months to years) |
| Parasites in blood | Yes | No* |
| Parasites in skin (sequestered is sub- | Yes | Yes |
| cutaneous microvasculature) | | |
| Transmission capacity (mosquitoes | Yes | Yes |
| bite skin) | | |
| Detectability with laser nanobubble | Yes | Yes |
| technology (in skin) | | |
| Detectability with RDT, microscopy | Yes | No** |
| and PCR (in peripheral blood) | | |
| Application | Clinical | Mass |
| | diagnostics | screening |

*residual parasites may present in blood in a very low density
**a high sensitivity PCR (<1 par/uL) may potentially detect residual parasites in blood when most of them are sequestered in tissue microvasculature

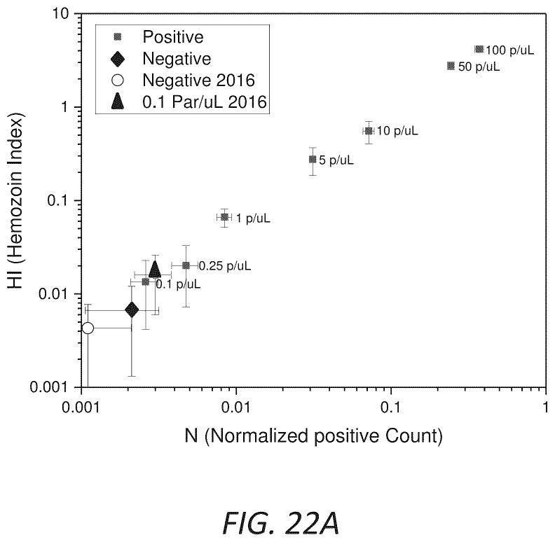

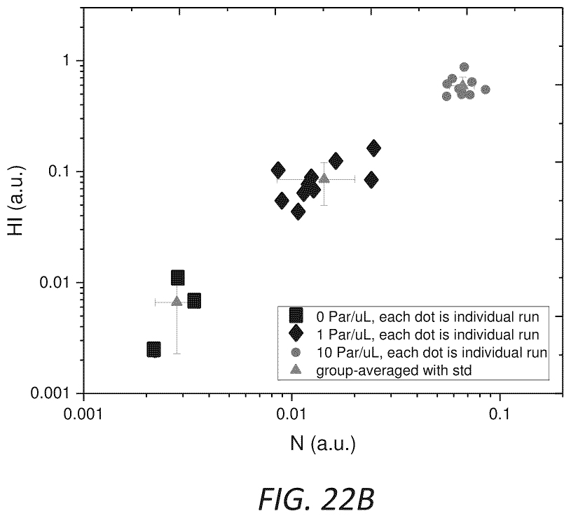

The differences observed above indicate that many local subjects from endemic area with malaria-negative blood still had skin which delivered hemozoin-generated vapor nanobubble-positive signals and hence potentially had parasites/hemozoin not found in their peripheral blood. The current malaria science suggests that there is no correlation between the malaria status of the peripheral blood and malaria transmission (which is related to the skin level of parasites, especially, gametocytes). Therefore, the asymptomatic data may support the hypothesis about high level of sequestered parasites and hemozoin in skin. Such cases may not represent clinical concerns, but they may represent transmission concerns even though such human subjects are not detected as malaria-positive with current standard malaria tests. That standard PCR analysis of most of such hemozoin-generated vapor nanobubble-positive subjects came out as negative. The non-invasive skin data suggest a possibility to use the hemozoin-generated vapor nanobubble method for the detection (and screening) of malaria transmission status of human subjects through a rapid and non-invasive hemozoin-generated vapor nanobubble test. The clinical studies suggest that a skin may be a better source for malaria detection and screening compared to the current standard, a peripheral blood, for the screening of malaria transmission. As shown in FIGS. 22A and 22B, an in-house in vitro blood model study (such as described above with reference to FIGS. 9A-9C) on P. Falciparum showed a parasite density detection threshold of at least 0.1 parasite/μL. The reproducibility of the test was approximately 10%.

### Comparison of Nanobubble Signals with Bulk Thermal Response Signals

As described above, examples of the malaria sensor disclosed herein can deliver laser pulses into the skin of a test subject (such as a patient) through an optical fiber. In some embodiments, the optical fiber can be a multi-mode optical fiber. The sensor can collect a spherical pressure pulse from a subcutaneous source, which can be the generation of a nanobubble around a malaria-specific nanoparticle, such as a hemozoin nanoparticle. The sensor can also amplify the electrical signal indicative of the pressure pulse to a level that can be analyzed.

The nanobubble-based malaria detection mechanism is different than the photoacoustic mechanism described above with reference to FIGS. 15A and 15B. While the nanobubble-based mechanism can produce a pressure pulse due to expansion and collapse of as few as a single transient vapor nanobubble, the photoacoustic mechanism produces weaker pressure pulses due to the thermos-elastic effect of tissue and/or blood in a bulk volume. The pressure pulse from the photoacoustic mechanism has lower amplitude than the nanobubble-generated pressure pulse. Although the photoacoustic mechanism may produce different thermal response signals for malaria-infected red blood cells and healthy red blood cells, the diagnosis of a malaria-positive status based on the differences in thermal response signals requires the presence of a larger number (at least more than one) of hemozoin nanocrystals. Therefore, the nanobubble-based mechanism can be better at reporting a malaria-positive status than the photoacoustic mechanism, although the latter may still be useful for reporting malaria-negative status.

Signals from experimental models and human subjects described below show that the transient vapor nanobubbles were directly detected in the skin during a non-invasive test and that the nanobubble signals correlate to the malaria-positive status.

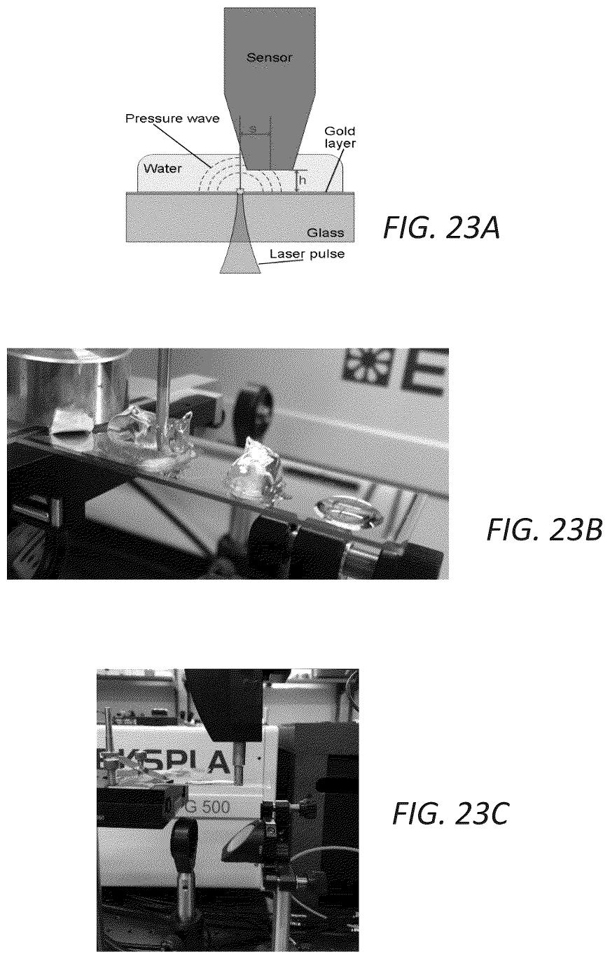

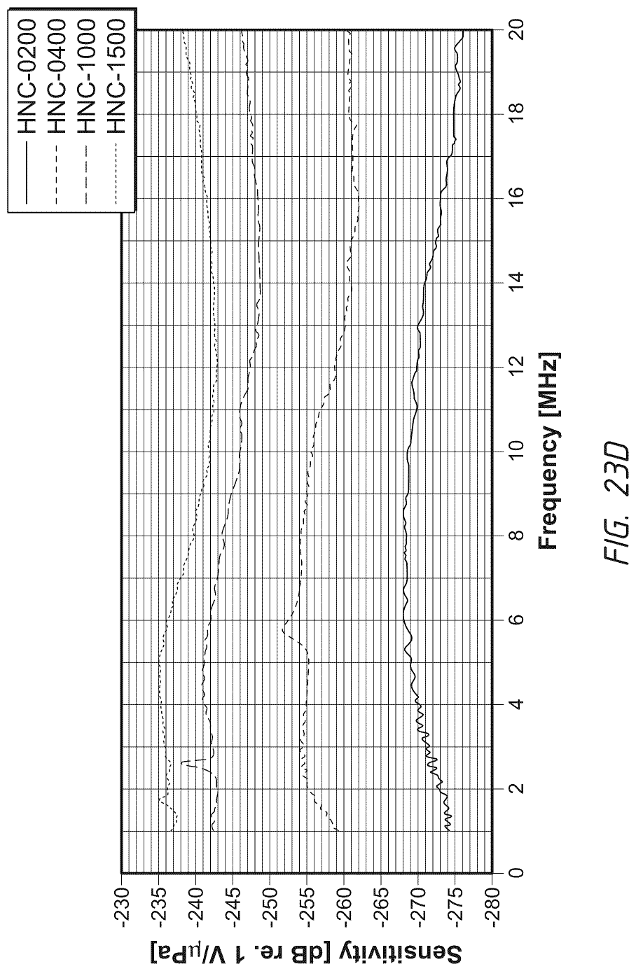

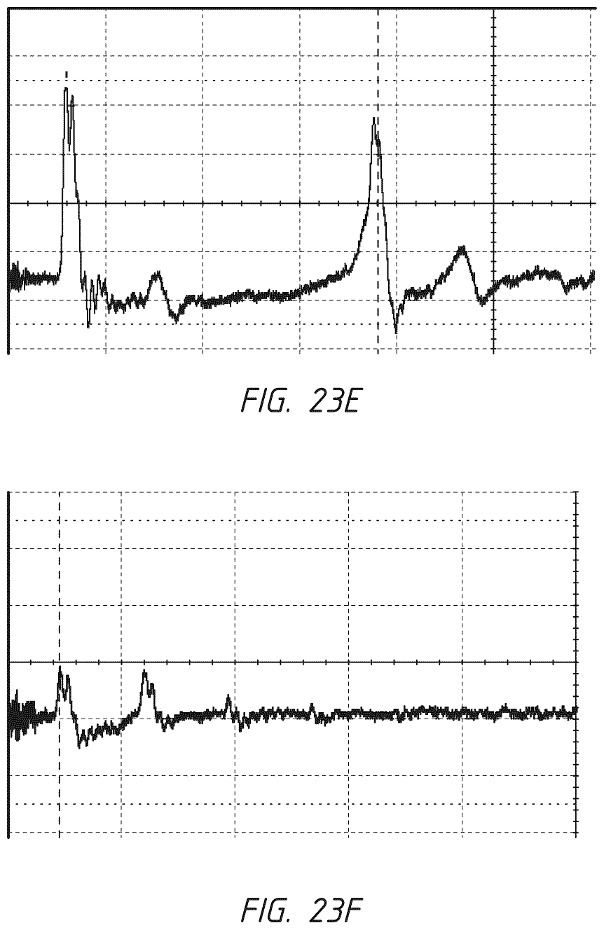

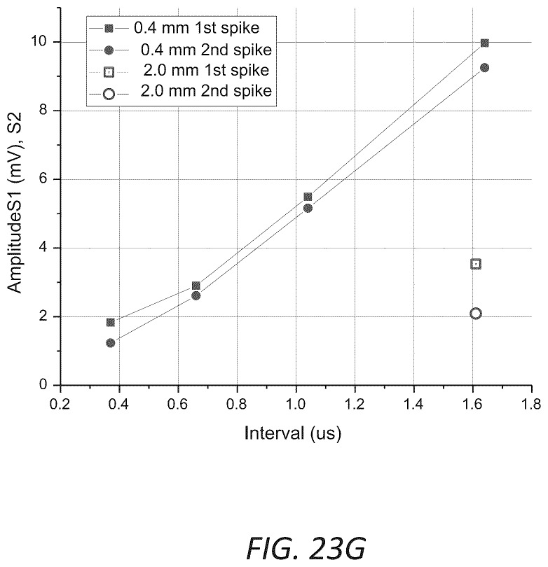

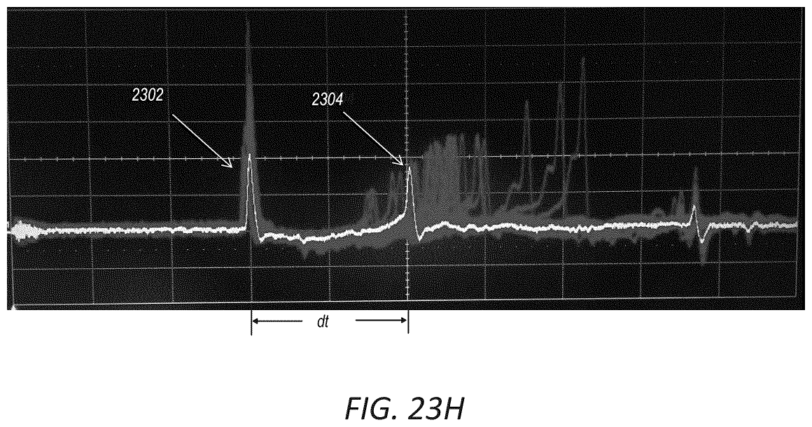

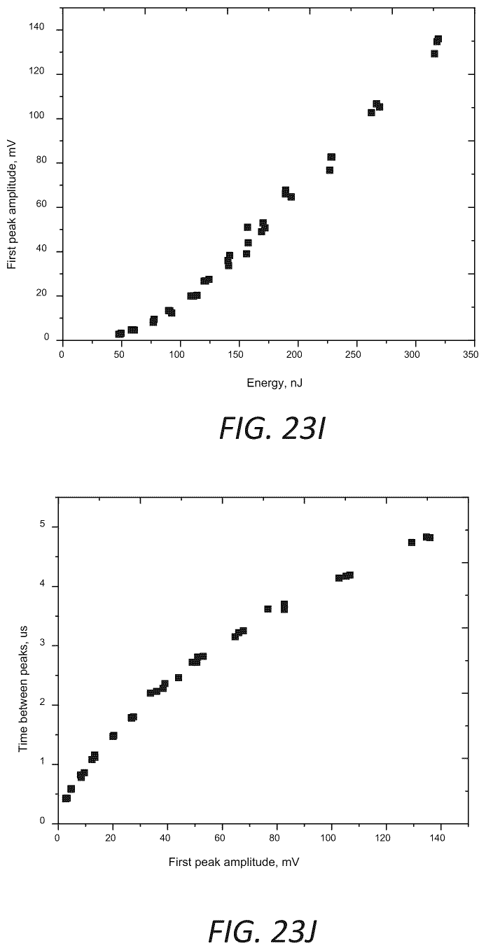

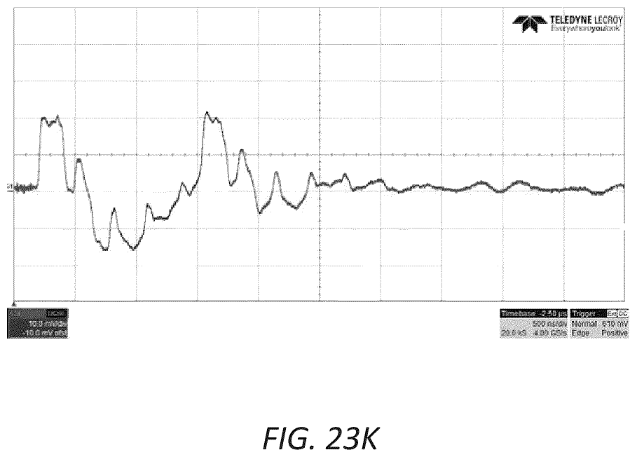

In an experiment using gold nanofim as a source of vapor nanobubbles as a reference model, such as shown in FIGS. 23A-23C, a gold nano-film of about 50 nm thick was deposited on a microscopic slide glass. The gold film model was used with three acoustic media on top of the gold film: water (speed of sound in water 1480 m/s), ultrasound gel, and dark skin with an ultrasound gel on top of the dark skin. The sample has been scanned to expose an intact gold surface to each next laser pulse. As shown in FIG. 23C, the malaria probe can be the probe shown in FIGS. 3A-3D. The setup as shown in FIGS. 23A-23C, coupled with the high stability and precision of the laser pulse delivery as disclosed herein, can ensure repeatability of photothermal effects in the uniform layer of gold film.

From the bottom of the glass slide, a single short 532 nm laser pulse was focused onto the gold film through the glass. This resulted in the generation of semi-spherical transient vapor nanobubbles in water and near the gold/glass surface. The maximal size (hence the lifetime) of a nanobubble was controlled through the energy of the laser pulse (through the amplification settings in a laser remote control). In this model, a vapor nanobubble acts as a point source which generates a pressure pulse with spherical wave front.

A calibrated reference hydrophone HNC 1000 or HNC 0400 (which may be the default choice) may be mounted, for example, at a slight angle above the gold surface. The hydrophone has a round element with the diameter 0.4 mm and the spectrum shown in FIG. 23D (which was obtained from third-party data). Its electric output was connected to a 20 db voltage gain 25 MHZ (hemozoin) band pre-amplifier, which was in turn connected to a 50 Ohm input of digital oscilloscope (which may be LeCroy X42 or a part of the malaria system disclosed herein). The height of the hydrophone was adjusted so the signal arrived in water in 0.3 us (1480 m/s×0.3 us=0.45 mm).

FIGS. 23E and 23F illustrate a returned signal in the gold-water model of vapor nanobubbles. Each division of the x-axis is 0.5 us. Each division of the y-axis is 2 mV. In FIG. 23E, the nanobubble has a lifetime of about 1.6 us and an energy level of about 23 mV. A transient vapor nanobubble assumes that a nanobubble expands and collapses in elastic medium, such as water. While mechanisms of the expansion and collapse are hydrodynamically similar, these stages are driven by different sources. An expansion stage is driven by the optically absorbed energy which, converted into a potential energy of a vapor, determines the maximal diameter of a nanobubble and the amplitude of a first spike in its signal, such as shown in FIGS. 23E-23F. A collapse stage is driven by a nanobubble environment, such as the pressure of a surface tension of the surrounding medium. This process results in accumulation of some energy which is released as a second pressure pulse (second spike) as shown in FIGS. 23E-23F. As shown in FIG. 23F, the smallest nanobubbles detected with this set-up revealed a lifetime of 0.35 us. A signal to background ratio can be estimated using the background peak-to-peak amplitude of 0.3 mV. The signal to background ratio was around 5 for the smallest nanobubbles detected.

As shown in FIG. 23G, the dependence of the positive component of the spike amplitude upon the nanobubble lifetime (measured as the time interval between the first and second spikes) showed (1) quasi-linear performance, in line with the theory for elastic medium, (2) close similarity of the amplitude of the second and first spikes, in line with the minimal losses of the nanobubble energy in the medium (the collapse signal was close to the expansion signal), and (3) lack of negative components in first and second spikes, in line with the theory for the medium with low elastic modulus and without generation of tensile stress. In addition, moving the hydrophone away from the source to 2 mm distance significantly reduced the signal amplitude.

FIG. 23H-J illustrates multiple traces of the generation of transient vapor nanobubbles using a calibrated 20 MHZ (hemozoin) hydrophone with a piezo element having a diameter of about 1 mm and at a hydrophone distance of 2 mm from the source. As shown in FIG. 23H, the transient vapor nanobubble typically results in a two-spike signal. The first spike 2302 is indicative of the nanobubble expansion and the second spike 2304 is indicative of the collapse of the nanobubble. FIG. 23I illustrates a relationship between the pulse energy and the first peak amplitude. FIG. 23J illustrates a relationship between the first peak amplitude and the time between the first and second spikes. The spike-to-spike time dt correlates to the laser pulse energy, which can be variable in some embodiments, and therefore, to the maximal size of the nanobubble. The results were similar to those shown in FIGS. 23D and 23E. However, the two-spike nanobubble signal may not be identifiable against the background of the thermal response bulk signals and/or the baseline signal, which may include distortions due to the internal functions of the malaria sensor. The 1 mm hydrophone was thus less sensitive in the detection of small nanobubble, probably due to the distance-induced attenuation of the signal as the pressure from the source in this model decreases inversely proportionally to the square of the distance to the hydrophone.

A ratio of the size of the piezo element in the hydrophone to the hydrophone-to-target distance can characterize a detection regime of the hydrophone as a far field (<1) and near field (≥1). In the far field, the sensor detects almost a flat wave so the detection conditions (angle of incidence and phase of the pressure pulse front) remain substantially equal across the whole surface of the sensor. In the near field, the sensor detects a spherical wave and the detection conditions significantly vary across the sensor surface. Further, any additional lateral shift of the sensor off the center axis (a factor that is unavoidable when the optical fiber is placed in the center and the piezo element has some lateral shift by definition) adds to the effective size of the sensor geometry. In the far field in FIG. 23H, the nanobubble signal correctly shows the pressure pulse shape and the timing: dual—spike signal as the nanobubble signature, and the time-delay between the two spikes quantifies the maximal diameter of the nanobubble. The spike half-width is about 40 ns (close to the speed limit of the sensor used). This narrow spike shows a typical pressure pulse, not a wave, both at the expansion and collapse stages of the nanobubble lifespan in water.

FIG. 23K illustrates a near field pattern with a hydrophone of 200 um in height and 1 mm in the detection regime. The signal in FIG. 23K illustrates broadening of both the first and second spikes. The expected increase in the first spike relative to the second spike amplitude is not shown (possibly due to dephasing) compared to a hydrophone of a 1.5 mm detection regime. Decreasing the sensor-to-target distance does not increase the signal amplitude because the angle of incidence increases with a relatively narrow pointing diagram for the piezo element. This reduces the acoustic sensitivity. The echo signals, caused by reflection of pressure pulse from the sensor surface and back from the gold surface (the sensor-to-target distance×2) shift to the main spikes as the sensor-to-target distance decreases.

In the near field, the nanobubble signal becomes distorted due to several reasons: (1) the angle of acoustic incidence becomes too high, which reduces the sensor sensitivity and the signal does not increase despite shorter distance to the target; (2) the piezo element becomes too large in size, causing significant dephasing (mismatch) in the pressure-to-charge conversion, which broadens the signal spike and further reduces its amplitude. The lateral shift of the piezo element relative to the pulse source (a feature generally unavoidable for the malaria sensor with the optical fiber in the center, as described above) causes further broadening of the signal spikes and the decrease in the amplitude of the signal spike. Those effects can be explained by the increasing angle of acoustic incidence and the resulting dephasing of the piezo-effect in a piezo element, especially if its size is relatively large compared to the sensor-to-target distance. Further, significant lateral shift reduces the difference between the nanobubble-specific and bulk background signals (which will be described in greater detail below), making it harder to differentiate those two signals.

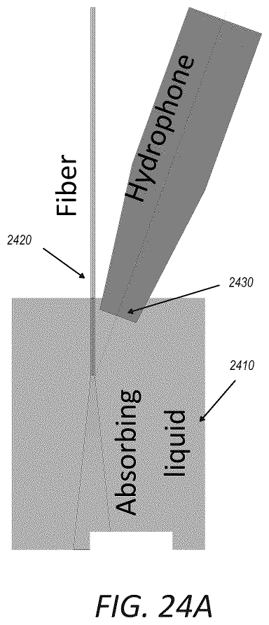

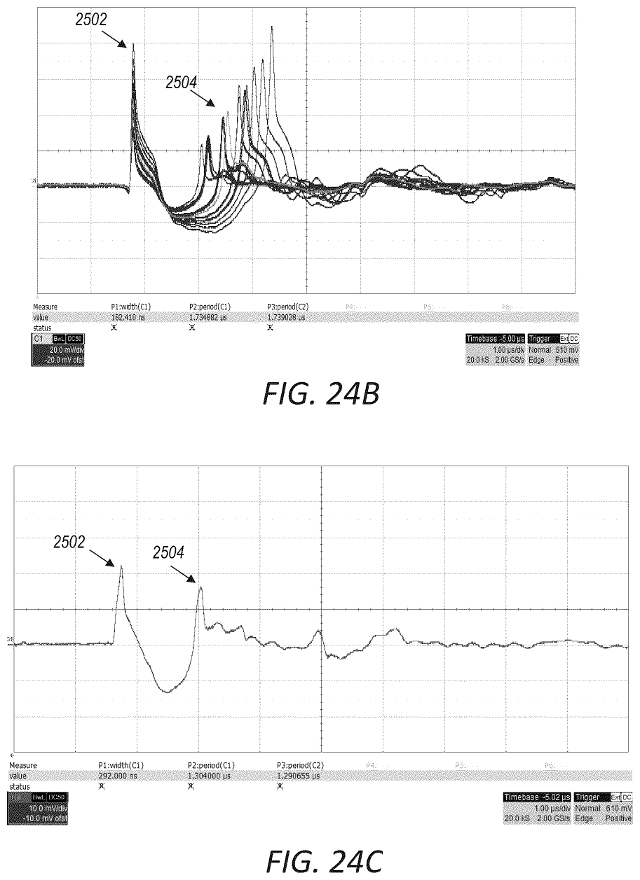

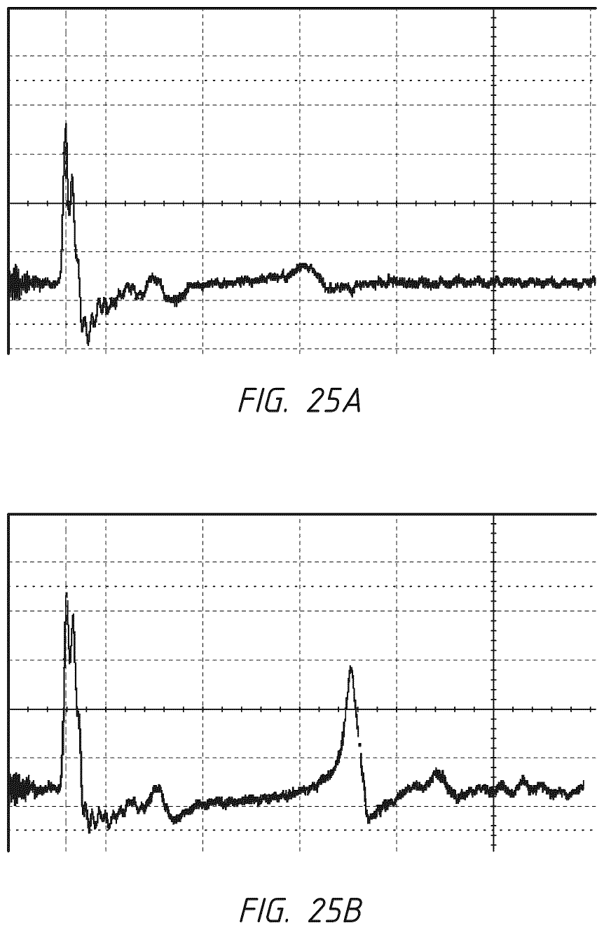

FIG. 24A illustrates an experimental model using a hydrophone 2430 placed in close proximity to an optical fiber 2420. As shown by the dash-dotted line in FIG. 24A, the hydrophone 2430 can be oriented to face a tip of the optical fiber 2420. The tip of the optical fiber 2420 can be directed at a hemozoin nanoparticle. The optical fiber 2420 can deliver pulsed laser energy into the air at about 16 J at a wavelength of about 671 nm, which is suitable for generating a vapor nanobubble around a hemozoin nanoparticle located no more than about 0.5 mm below the skin surface of a human subject. As described above, the ratio of the size of the piezo element in the hydrophone to the piezo-to-target distance can characterize a detection regime of the hydrophone as a far field (<1) and near field (>1). In the illustrated model of FIG. 24A, the distance between the hydrophone tip, where the piezo element is located, and the tip of the optical fiber can be about 2 mm. The ratio of the size of the piezo element to the piezo-to-target distance is about 0.5, which characterizes the hydrophone as having a far field detection regime.

In FIG. 24A, the tips of the optical fiber 2420 and the hydrophone 2430 can also be submersed in a liquid. As shown in FIG. 24A, the liquid can be an absorbing liquid 2410 having a brown color. The absorbing liquid can be a solution having iodine, potassium iodine, ethanol, and/or water. The absorbing liquid can have an absorption coefficient of about 1.9 cm⁻¹. The absorbing liquid can simulate skin pigmentation in a human subject.

Three testing models can be constructed using the optical fiber-hydrophone arrangement illustrated in FIG. 24A and/or an example malaria sensor disclosed herein. The optical fiber-hydrophone arrangement and/or the malaria sensor can be applied to (1) a hemozoin nanoparticle without the absorbing liquid to detect a pulse due to generation of a vapor nanobubble only (“nanobubble model”); (2) the absorbing liquid without a hemozoin nanoparticle to detect a pulse due to photoacoustic mechanism only (“bulk model”); and (3) a combination of the hemozoin nanoparticle and the absorbing liquid to detect pulses due to both the vapor nanobubble generation and the photoacoustic mechanism (nanobubble and bulk model”).

FIGS. 24B and 24C illustrate example nanobubble signals received by the hydrophone in the nanobubble model. Similar to the signal in FIG. 23J, the nanobubble signals have two spikes corresponding to the expansion 2502 and collapse 2504 of the nanobubble. However, as described below, the two-spike nanobubble signal may be not identifiable at the background of a bulk signal due to the thermal response of the tissue of the human subject.

For the water model of vapor nanobubbles, the transient vapor nanobubbles in water can produce a two-spike signal with the corrected spike amplitude in 2-3 mV range (with a 10-fold amplification of the hydrophone) for a nanobubble of 1 microsecond lifetime, which corresponds to the maximal diameter of 10-20 um approximately. The nanobubble expansion and collapse are nearly symmetrical, the second spike generated by the collapse can be close in amplitude to the first spike caused by expansion and such nanobubbles create no tensile stress in water.

A nanobubble in skin can be modeled with a human skin sample surrounding the nanobubble generated by the gold-water model described above. The test sample included a gold nano-film deposited on the microscope slide glass and the human skin sample on top of the gold film, in physiological solution or in water. The skin sample was dark human skin, 250 um thick, and including epidermis and dermis. A drop of water was deposited on the sample to couple the sample to the hydrophone. In this model, a nanobubble formed by optical excitation pulse as described above with reference to FIGS. 23A-G expanded and collapsed in the skin, specifically, the dermis, its pressure pulse propagated through the skin towards a hydrophone at a speed of sound of 1620 m/s. The acoustic detection using a hydrophone was also as described above, except that there was a need for a ×0.5 correction of the spike amplitude to compensate for a dual pressure pulse.

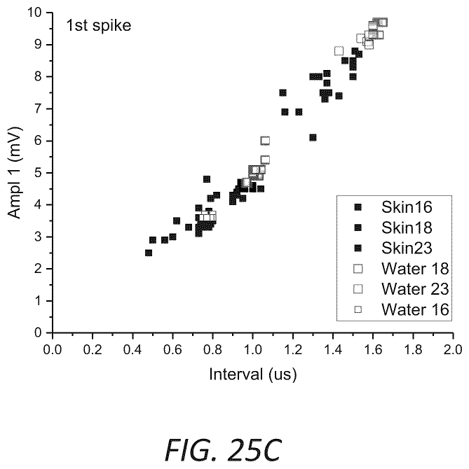

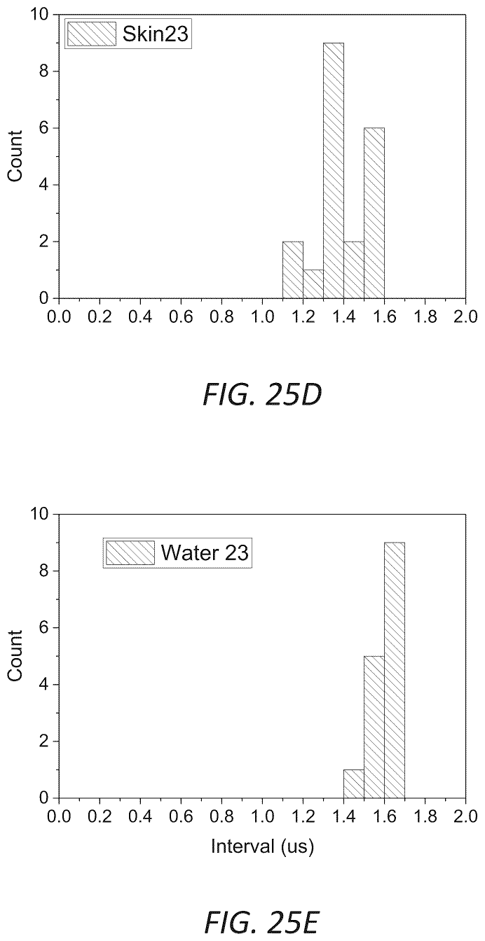

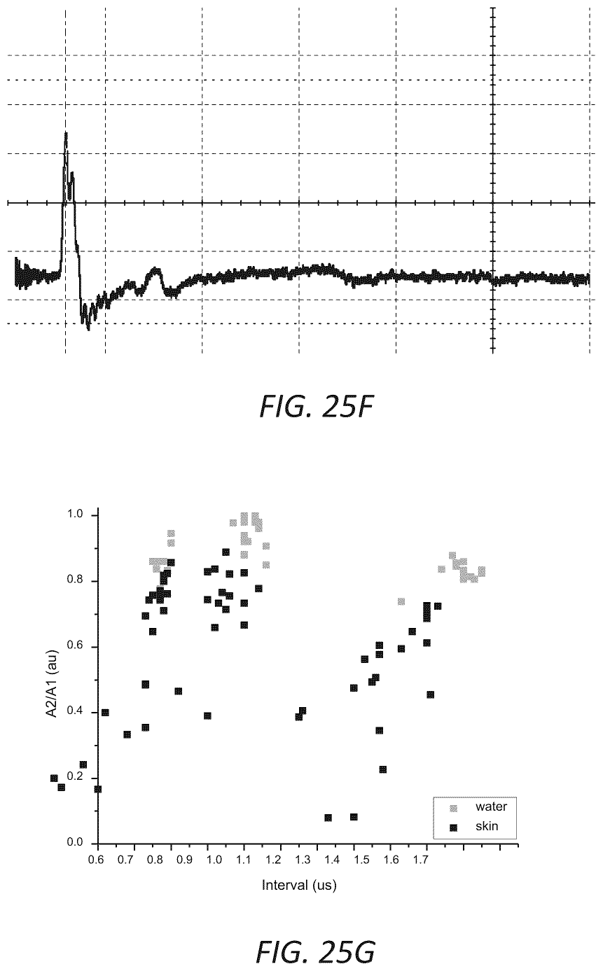

After a sample of dark human skin of 250 um thickness was placed on top of the glass, the energy deposition and vaporization processes have not changed because the laser-target interaction and target properties have not changed. However, as shown in FIG. 25A, under identical optical energies as shown in FIG. 23E, the signals have changed dramatically. The nanobubble lifetime has decreased. The first spike amplitude decreased slightly. The second spike amplitude, shape and time position revealed a significant decrease which has varied in a broad range. The amplitude of the second spike amplitude and lifetime have decreased, and the spike width has increased.

FIG. 25B illustrates a signal from a quantitative study of the skin vs. water, which included the analysis of the first spike shape, amplitude and life, and the second spike amplitude and shape. Tensile stress was observed as the negative component in the spikes in signals with significant damping of the second spike caused by the collapse of the nanobubble, such as shown in FIG. 25A. This tensile (negative) component was much smaller in signals with a good second spike, that is, without significant damping, such as shown in FIG. 25B. The skin has a much higher elastic modulus (much stiffer) than water and can develop a tensile stress during the propagation of the pressure pulse. A tensile stress may be one of the main causes of the structural damage for a skin at microscale.

FIG. 25C illustrates the dependence of the positive amplitude of the first spike (without correction) upon the nanobubble lifetime. Numbers in legend indicate a laser pulse energy. As shown, a much higher spread of signals can be observed after skin was added to the model. FIGS. 25D-25E illustrate histograms of a nanobubble life for water and skin under identical conditions.

The nanobubble energy is dissipated during the expansion stage in the skin model. As shown in FIGS. 25C-25E, the amplitude of the first spike and the lifetime have decreased in the skin sample compared to those in water. Such energy loss was not observed for water. This energy loss can be caused by the plastic deformation in skin (vs elastic in water), formation of cracks, and viscous losses, all of which can be caused by the mechanical response of the skin to the nanobubble expansion. This energy loss has influenced the first spike, and caused the deviation of the nanobubble signal parameters from those in water.

The skin also has significantly influenced the collapse of a nanobubble (the second spike), sometimes up to its full damping sometimes as shown in FIG. 25F. The disappearance of the second spike may mean the nanobubble did not collapse normally. Such severe damping of the collapse may be caused by plastic (vs elastic in water) deformation of skin during the nanobubble expansion so quasi-permanent air voids and cracks emerged in skin where a nanobubble was generated.

FIG. 25G illustrates dependence of the second spike to first strike amplitude ratio on the lifetime of a nanobubble generated in the gold-water and gold-water-skin models. The quantitative analysis of the compromised collapse of nanobubbles in skin can be seen for three laser energy levels: the second spike to first spike amplitude ratio, A2/A1, for water did not depend upon the nanobubble lifetime and can be highly reproducible in the range 85-100%. In skin, the ratio has dropped significantly and was very unstable (due to variation of skin properties from location to location since new location was used for each next signal). In addition, the detection of the second spike could be compromised due to its broadening. This effect was caused by heterogeneities in the skin, such as the cracks that could have been induced by the expanding nanobubble.

Table 9 below includes statistics for signals in skin and water under identical laser excitation and acoustic detection.

TABLE 9

| | | Dark skin | Dark skin (250 um) |
|---|---|---|---|
| | | (250 um) | with Plasmodium |
| | Single | on top | Falciparum |
| | nanobubble | a single | parasites |
| | in water, | nanobubble, | underneath (6/13/18, |
| | e = 23, | e = 23, | FIG. 6), n = 53, |
| Sample | n = 15 | n = 20 | Section 4 |
| 1^{st} spike amplitude, | 4.7 ± 0.13 | 3.9 ± 0.34 | Not analyzed due to |
| corrected, mV | | | the background |
| | | | (37.6 mV) |
| 2^{nd} spike amplitude, | 3.9 ± 0.2 | 2.0 ± 1.4 | 2.3 ± 1.4 |
| mV | | | |
| 2^{nd}/1^{st} spike ratio of | 83 ± 3 | 49 ± 0.19 | Not analyzed due to |
| amplitudes, % | | | the background |
| Lifetime, us | 1.6 ± 0.05 | 1.4 ± 0.1 | 1.4 ± 0.6 |

It is possible to expect irreversible local structural changes in the exposed skin after each nanobubble. A small air bubble may remain in skin for up to 30 seconds. Such changes may cause (1) delivery of new parasites into the exposed volume after the local implosion of the skin when the void created by a nanobubble finally collapses, and (2) a change of the local optical scattering properties of the skin (so the detection of “after-bubble” may be an option detection of a parasite).

It is also possible that at a smaller scale of hemozoin-generated vapor nanobubbles (the maximal diameter 10 um or less), the plastic deformation and associated damping of the nanobubble collapse may be the minimal and thus the quality of the second spikes may improve. However, such second spikes may be located close in time to the background spike (<300 ns interval), and may have a small amplitude. Such second spikes may require more precise detection.

Accordingly, in skin (unlike water), a more reliable signal for a transient nanobubble may be the first spike associated with its expansion. The second (collapse) spike has strongly damped behavior and appears to be unstable due to the plastic deformation of skin and viscous damping. Further, the simultaneous generation of several closely located nanobubbles of different maximal size (and hence lifetime) may amplify the first spike but may broaden/diffuse the signal of the second spikes (since individual nanobubbles collapse in different time).

FIG. 26A illustrates optical excitation and acoustic detection of parasites through the skin via a schematic representation of the test model. The sample included several layers of dark (African type) skin, such as shown in FIG. 26D, with the high level of melanin and gel or water as an optical and acoustic coupling media. Intact skin samples had no parasites. Parasite-positive samples had cultured Plasmodium Falciparum human malaria parasites (gametocytes), which were deposited onto the dermis layer and then were covered with a sample of upper skin (of epidermis and dermis, 250 um total). The deposition process and the sample preparation in general were optimized to achieve single residual parasites located between two layers of dermis and within the laser-exposed skin volume. Ultrasonic gel or water was used to prevent sample from drying during the experiment.

FIGS. 26B and 26C illustrate positions of the optical fiber and a tilted hydrophone in the experimental setup for studying optical excitation and acoustic detection of parasites through the skin. Optical excitation was performed via the multi-mode optical fiber with a 105 um core diameter, 2 m length. The fiber was brought in contact with the skin surface. Laser pulse (671 nm, 220 ps pulse duration, energy 15 uJ) was applied. Three to four laser pulses were applied to each skin location. Next, the sample was scanned in order to expose a new location to the next set of identical laser pulses. In this model, the laser pulse propagated through the skin surface and through the layer of melanin and deeper into dermis where parasites were placed.

Acoustic detection was performed using a HNC 0400 (0.4 mm) reference hydrophone (FIGS. 26A-C) which has been brought as close as possible to the wall of the optical fiber and to the top of skin surface. A parasite-to-hydrophone distance was approximately 0.4-0.5 mm. A sufficient acoustically similar material was placed underneath the skin sample, 2-3 mm of skin dermis and 6 mm of acoustically similar rubber, in order to prevent acoustic reflections and minimize echo signals.

As shown in FIGS. 26E and 26F, the intact skin sample revealed a background signal: a single high amplitude bipolar spike caused mainly by melanin. The amplitude of the background at the time >0.5 us was 1-2 mV. The presence of a negative component indicated both a tensile stress being induced by melanin and a higher stiffness (higher elastic modulus) of the skin compared to water. The positive spike had a single maximum. The amplitude of positive component of the spike was almost one order of magnitude higher than that of the first spike of a single nanobubble, such as shown above in FIG. 25A. Due to the high amplitude and the negative component after the spike, the baseline after the spike had a “ripple” extending to about 600-700 ns after the peak of the spike position. A slight decay of the amplitude of the background spike was observed at the same location for the second and next laser pulses (FIGS. 26G and 26H; Table 10 below). As shown in FIGS. 26G and 26H, both the positive and negative components decrease for the 2nd laser pulse. The signal was reproducible.

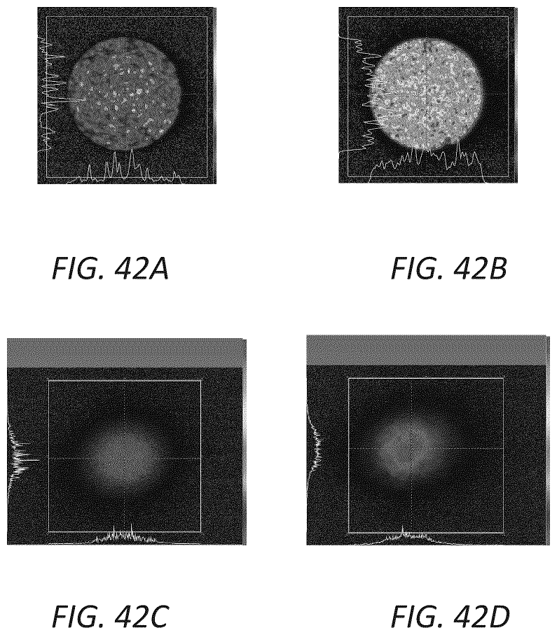

As shown in FIGS. 261 and 26J, signals of skin with Plasmodium Falciparum parasites revealed several features different from signals of intact skin: (1) the second spike at the time interval typical for a collapse of transient nanobubble (FIGS. 261 and 26J), (2) disappearance of the second (collapse) spike upon exposure of the same skin location with next laser pulses at the same time position where the initial second spike was observed (FIG. 26J) and (3) doubling of the first spike in many (but not all) locations (FIGS. 26K and 26L). Signals obtained from parasite skin had components typical for vapor nanobubble signals in skin such as described above. After 3-4 laser pulses to the same location, the collapse spikes disappeared and the signal became similar to that of an intact skin. At the same time, a significant decrease in the amplitude of the first spike was observed (see Table 10 below). The first spikes included both the background and a nanobubble component. The dual first spike might have been caused by a depth difference for melanin and parasites (although the location of melanin is not a plane layer at specific skin depth). With the 0.4 mm aperture of the detector located at 0.3 mm from the skin surface, and with the aperture of the source of the background signal being approximately 0.1 mm, the background spike would naturally broaden. This, in turn, would decrease the time interval for signals from the melanin layer and parasites (located 100 um deeper). Hence the time interval observed between two peaks in the dual first spike was only 25-35 ns and did not allow for monitoring the depth of parasites. The negative component of the first spike was observed and it was quite strong, thus indicating a tensile stress in a relatively stiff (high elastic modulus) medium compared to water. The sample preparation could not guarantee that each laser pulse (each new location) would include malaria parasites. As a result, sometimes signals similar to those for intact skin were observed. In general, a shape of signals with a second spike was similar to that of signals of nanobubbles in gold-skin model (FIGS. 261 and 26J vs. FIG. 25A, Table 10 below) and hence the signals detected were attributed to vapor nanobubbles.

The following metrics were observed for the signals in the model shown in FIGS. 26A-26L. The first spike has a permanent time position (begins at around 300 ns). This spike had a component caused by the expansion of vapor nanobubble and hence such component was determined by the laser fluence, size of hemozoin and other laser- and parasite-related properties. Several typical features were observed. The positive component decreased after each pulse. That is, the amplitude decreases (the decay effect). The negative component did not change, its amplitude remaining constant (the tensile stress is thus present). The positive component doubled in this model (a possible separation of the background signal and the nanobubble expansion signal).

For the second spike, the time interval (lifetime) observed was in the range of 0.8-2.8 us. The second spikes were not observed below 0.7 us, maybe due to the ripple in the baseline. This spike was caused by the collapse of a vapor nanobubble and hence was determined by the mechanical properties of the skin. The skin influence on the second spike in parasite-treated skin appeared to be very similar to that observed in the gold-skin model. Several typical features were observed. In the initial time position, the amplitude of the signal decreases to 0% (the spike disappeared) when the same location is exposed to the second or next laser pulses. Additional spikes were observed at shorter time intervals (the “move-in” effect), which was associated with the generation of smaller vapor nanobubbles as consecutive laser pulses were applied to the same skin location. To quantify parasite-specific features, eight metrics were introduced:
A1: Positive amplitude of the first spike, individual signals;
D1: Decay of the first spike, the level of the residual amplitude in response to the second laser pulse vs the first laser pulse, two signals; Incidence of the dual 1st spike, %: group of signals;
T: Time interval for the 2nd spike, measured between the second (largest if several) and first spikes,
A2: Amplitude of the largest second spike, individual signals;
D2: Decay of the second spike, the level of the residual amplitude in response to the second laser pulse vs the first laser pulse, two consecutive signals;
N2: Incidence rate of the second spike (threshold peak-to-peak amplitude of the baseline in intact skin), %, group of signals; Move-in effect of the second spike(s), %, group of signals.

These metrics were analyzed for intact and parasite-treated dark skin of 250 um thickness, all in response to 671 nm 15 uJ laser pulse delivered via the optical fiber with a 105 um core. Table 10 illustrates a comparison of intact and parasite-treated skin samples, dark, 250 um thick.

TABLE 10

| Signal metric | Intact (n = 27) | Parasites (n = 53) |
|---|---|---|
| Amplitude Spike 1 | 30.5 ± 4.8 | 37.6 ± 6.7* |
| Decay, Residual | 88.2 ± 7 | 74.8 ± 10* |
| Amplitude Spike 1, % of | | |
| the signal 1^{st} laser pulse | | |
| Interval Spike 1-2 | 0.22 ± 0.5 | 1.36 ± 0.6 [0.8-2.5]* |
| Amplitude Spike 2 | 0.31 ± 0.68 | 2.26 ± 1.4* |
| Decay, Residual | 84.3 ± 32 | 15.4 ± 32* |
| Amplitude Spike 2, % of | | |
| the signal 1^{st} laser pulse | | |
| Spike 2 incidence, % | 18.5 | 89 |
| Dual 1^{st} spike, % of signals | 0 | 50 |
| “Moving inside” 2^{nd} spikes, | 0 | 58 |
| % of signals | | |

The detailed analysis of the first spike (background signal and the effect of the expansion of a nanobubble) is provided herein. Regardless the shape of the peak (a single or dual peak), its amplitude in response to the first laser pulse and the decay were different in intact and parasite-treated skin (FIGS. 26M-260). For the first laser pulse, the positive amplitude of the first spike increased in parasite-treated skin while the residual level during the second laser pulse (decay metric) decreased. Up to 6 signals in the parasite-treated group did not show second spikes and might not generate any nanobubbles (these locations may have been parasite-free). The first spike data suggests that the pressure pulse of expanding nanobubble adds to the background pressure by increasing the amplitude of the first spike and, in slower detectors, causing a spike time-shift to the right. During the next laser pulse, nanobubbles were smaller or disappeared resulting in a significant decrease in the positive component of the amplitude of the first spike (see the decay effect shown in FIG. 26M).

The detailed analysis of the second spike (the effect of a nanobubble collapse) is provided herein. In FIG. 26P, the amplitude measured during the first laser pulse was plotted vs the time interval. Amplitudes for the gold model were corrected with the coefficient 0.5 to account for the detection of doubled pressure pulses due to the echo from the glass in this model. The signals were obtained with a 0.4 mm hydrophone. The deviation from the nanobubble water model might have been caused by the effect of skin on (1) the nanobubble collapse and (2) propagation of the pressure pulse through the skin. For the interval of 1.6-1.8 us, the gold model signal amplitude was 4.6 mV (corrected) while the average (over 11 signals in this interval range) was 2.6 mV (57% of the amplitude in water, see Table 10).

The main features of FIG. 26P (also, compared to FIG. 25C for the gold-water and -skin model) include the following. Signals in skin for nanobubbles (the gold model) and parasites (skin model) show similar trend, in line with that for a nanobubble collapse in a medium with plastic deformation. The increase of an amplitude with the lifetime of a nanobubble was observed but it was less reproducible compared to the data for the water sample. Signals in skin (in both models) show similar amplitudes to signals in water model of an “ideal” nanobubble. Compared to water, signals in both skin models show poor reproducibility of the spike amplitude.

FIGS. 26Q and 26R illustrate the decay of the second spike amplitude after the second laser pulse is applied to the same skin location. The signals were obtained with 0.4 mm hydrophone. The second spike disappears in response to the next laser pulse or moves closer to the first spike (at short time interval from the first spike) for the parasite-treated skin while a similar part of a signal of intact skin experiences no changes (remaining, in fact, a baseline). This effect is in line with the nature of hemozoin-generated vapor nanobubbles. The cluster of hemozoin crystal is disintegrated by the impact of the first nanobubble so the second laser pulse produces much smaller or no vapor nanobubble from the same hemozoin target, which is no longer as large and clustered as before the first laser pulse.

In summary, signals obtained from a human skin sample with residual parasites had shape and metrics similar to those of the gold-skin nanobubble model. This similarity appears to validate the signals detected in a parasite-treated skin as signals of vapor nanobubbles. The properties of two main nanobubble signal components, the first and second spikes in the human skin sample are summarized in Table 11A.

TABLE 11A

| Nanobubble signal | | |
|---|---|---|
| components | 1^{st} spike | 2^{nd} spike |
| Relation to a transient | Expansion | Collapse |
| nanobubble | | |
| Nanobubble vs background | Almost coincides in time | Well-separated in time for |
| signal of melanin in dark skin | Its amplitude is 5-20% of the | large nanobubbles with |
| | background signal | lifetime >0.8 us (max |
| | | diameter about 10 um) |
| Acoustic detectability | Poor direct detection - | Poor with the current sensor- |
| | disguised by a much stronger | unstable in the skin unless a |
| | background signal | superior sensor with the high |
| | Direct detection requires | acoustic sensitivity is used |
| | better temporal resolution | |
| | Indirect detection is | |
| | possible through a time-shift | |
| | and the amplitude decay of | |
| | one integrated peak | |

As shown in Table 11A, vapor nanobubbles are generated in skin around Plasmodium Falciparum human malaria parasites in the human dark skin. Plastic and viscous properties of the skin can dampen the collapse stage of the nanobubble, the effect resulting in an unstable second spike associated with the dampened collapse. The acoustic detection of parasite-generated nanobubbles disclosed herein may be more effective for relatively large nanobubbles with the lifetime above 0.8 us, which corresponds to the maximal diameter of a nanobubble about 10 um. The background signal due to optoacoustic emission by melanin can be high and create one of major challenges in the generation and detection of nanobubbles around malaria parasites in a dark skin.

Several options are available for improving the nanobubble method disclosed herein. It may be possible to modify optical excitation in the way its fluence at the melanin depth is reduced (for example, via side launch of the focused launch of a pump laser pulse as shown in FIG. 26S). The focused excitation beam reduces the background and improves the parasite signal. The shift of the background source (melanin) from the detection axis (and, generally, from the angle of acceptance) of ultrasound sensor reduces the background signal. As the melanin requires a lower fluence than the malaria parasites for optical excitation, having acoustic pulses from the melanin and the parasite arriving at the acoustic detector or sensor at different angles may suppress the melanin signal.

The propagation of the excitation laser beam and its absorption in melanin-rich dark human skin was modeled with a computer program. The skin was modeled through 7 layers to describe stratum cornea, epidermis with the layer of melanin and upper layers of dermis. Optical scattering of the excitation beam by each skin layer and its optical absorption were analyzed for specific wavelength of the excitation laser pulse. The program and the model were used to compare the propagation of the excitation laser beam in water and in human dark skin (FIG. 34A-34B). The side- and focused launch resulted in an axial position of the focal point in skin shifting by +50, and a diameter of the focal zone for the NB generation becoming ten times larger in skin than in water.

Next, the monte-Carlo simulation method has been applied to analyze the propagation of individual photons through the skin under direct delivery from the optical fiber and the focused deliver from the top and from the side (see Table 11B below).

TABLE 11B

| | | | The side-launched |
|---|---|---|---|
| | Standard | The focused beam, | focused beam, |
| | optical fiber, | straight, incident | incident |
| Delivery | 100 um core | diameter 300 um | diameter 250 um |
| Skin | See FIG. 35A | See FIG. 35B | See FIG. 35C |
| model | | | |
| Deposition of | See FIG. 36A | See FIG. 36B | See FIG. 36C |
| the optical | | | |
| energy | | | |
| The fluence | See FIG. 37A | See FIG. 37B | See FIG. 37C |
| delivered, | | | |
| The area of | | | |
| the | | | |
| generation of | | | |
| vapor | | | |
| nanobubbles | | | |
| for the | | | |
| threshold of | | | |
| 18 mJ/cm2 | | | |

As can be seen from the results of the computational modeling, the background signal amplitude-driving laser fluence at the level of melanin in skin decreases in both cases of the focused beam compared to that for the standard launch with an optical fiber. Further, the fluence of the excitation beam at the level of possible location of malaria parasites increases (compared to that for the standard launch with an optical fiber) in both cases of the focused launch. This computational model has been used to design the experimental model with dark human skin and malaria parasites in the skin, and to compare the standard launch of the excitation laser beam and the side-focused launch.

The side launch of the excitation laser pulse spatially decouples the source of the background signal (melanin in human dark skin) from the source of malaria signal by shifting the source of the background pressure pulse out of the optimal detection angle of acceptance of the acoustic detector (FIG. 39A). FIG. 39B shows that the release of the energy absorbed by melanin is shifted to the left relative to the array of acoustic detectors which are tuned into the area in the focus of the excitation laser beam (the focused beam).

The goal of the delivery of the pump beam is to minimize the optical fluence at and associated thermal impact of the background skin (especially, upper level-located melanin) and to maximize the optical fluence at the depth of parasites. This is achieved by focusing the probe beam and by launching it as some angle so the background skin volume is spatially decoupled from the skin volume with hemozoin and or parasites. FIG. 40 illustrates an example experimental example of such spatial decoupling. The experimental prototype in FIG. 40 was designed to side-launch and focus the excitation laser beam. Although the melanin layer produces some photothermal and photoacoustic background signals, the source of such signal is shifted away from the axis of the detection of the hemozoin-generated vapor nanobubble signal and the amplitude of the background signal is reduced due to much lower optical fluence of the excitation laser beam at the level of the background (melanin, usually located in the upper skin layer while parasites are located 100-300 um deeper in skin). FIGS. 41A-41C are images of the excitation beam as it was launched into water from the right side show the intensity profile at the different depths.

Such spatial decoupling of the target (hemozoin) and background (melanin) was validated in dark human skin (rich with melanin) and human parasites placed at the depth of 250-300 um below the skin surface. Ultrasonic signals were compared for the background and hemozoin-generated vapor nanobubble to those obtained under the regular optical delivery through the flat 105 um core optical fiber.

Compared to a standard or direct optical fiber launch in intact dark skin (FIG. 26U) and parasite-infected dark skin (FIG. 26W), the signals from a side focused launch in the intact dark skin (FIG. 26T) and the parasite-infected dark skin (FIG. 26V) showed a 3-4 fold suppression of the background signal due to the melanin in the skin and a 2-fold increase in the hemozoin-generated vapor nanobubble signal amplitude. It may be possible to improve the acoustic sensitivity and temporal resolution of acoustic detector in order to differentiate the nanobubble signals from those of melanin. It may be more desirable to detect a nanobubble or/and its pressure pulse as close to its origin as possible. It may be possible to increase the statistics of the skin locations probed. Additional detail is provided in Table 11C.

TABLE 11C

| | Skin sample | | | |
|---|---|---|---|---|
| | Intact | | Parasites | |
| | Side | Fiber, | Side | Fiber |
| Laser launch | focused | standard | focused | standard |
| Positive amplitude | 8.9 ± 1.8 | 28.4 ± 9.3 | 12.8 ± 4.2 | 24.3 ± 6.3 |
| of the 1^{st} spike for | | | | |
| the 1^{st} laser pulse | | | | |
| Incidence of the | 0 | 7 | 64 | 46 |
| 2nd spike, % | | | | |
| Positive amplitude | 0 | 0 | 5.0 ± 2.1 | 2.8 ± 1.3 |
| of the 2^{nd} spike | | | (hemozoin- | (hemozoin- |
| | | | generated | generated |
| | | | vapor | vapor |
| | | | nanobubble) | nanobubble) |

Additional improvement of the optical delivery was achieved by suppression so called “hot spots” in the pump laser beam (which cause additional false-positive signals generated by melanin). Such suppression was achieved by homogenizing the laser beam intensity inside multi-mode optical fiber by increasing its lens from 1-2 μm to 12 m (see Table 11D).

TABLE 11D

| | Standard fiber | Long optical |
|---|---|---|
| | length | fiber, 12 m |
| Optical intensity profile at | FIG. 42A | FIG. 42B |
| the tip of the optical fiber | | |
| Optical intensity profile at | FIG. 42C | FIG. 42D |
| the bottom of 250 um dark | | |
| human skin | | |

A comparison of the skin model and the field human data will be described below. FIG. 27A illustrates schematically an experimental setup for detection of malaria parasites in skin with a slow-speed detector, such as the malaria probe examples disclosed herein (for example, the probe in FIGS. 3A-3D). The skin model sample can include dark skin of 250 um thickness, facing down, with Plasmodium Falciparum parasites on the top or the bottom of the skin. A 1.0 mm reference hydrophone was placed on the top of the model at 2 mm distance in order to co-register signals with the malaria probe. In the human studies, the samples can include dark skin with Plasmodium Falciparum malaria infection and medium dark skin with Plasmodium Vivax malaria infection. The same malaria probe as the skin model was used.

The propagation of acoustic pulse from the VNB through the skin to the acoustic detector located at the skin surface is modeled. For the focused beam of 300 um diameter at the skin surface, a propagation and detection of a single acoustic pulse emitted was estimated, for example, during the expansion of a vapor nanobubble around malaria parasite as shown in FIGS. 38A and 38B.

Optical excitation used the same laser pulses as shown with reference to FIGS. 23A-23C. Single laser pulses of the energy 15 uJ were applied. The skin sample was scanned across the sensor surface in order to avoid double laser exposure of the same location in the sample. The conditions were similar to those in human field studies.

The malaria sensor probe can include a low-speed sensor with a relatively poorer temporal resolution and poorer acoustic damping, compared to those of the 0.4 mm hydrophone). Co-registration was achieved with the 12 MHZ (hemozoin) reference hydrophone (1.0 mm element, Onda HNC1000) on top of the sample. Due to acoustic reflections from a hard surface of the malaria probe, the reference hydrophone detected both direct signals and their echoes. The only echo-less signals were those originated from the surface of the malaria probe. For the malaria probe, the detection conditions were similar to those in human field studies.

FIG. 27B illustrates example bulk signals from the hydrophone in the bulk model. The signal represented by the solid line is from the hydrophone and the signal represented by the broken line is from the example malaria sensor. The sensor signal can have a single spike 2601 followed by distortions or non-flat regions 2605 in the baseline due to internal functions of the sensor. The single spike 2601 is due to the thermal response of the absorbing liquid to the laser pulses. The hydrophone signal can have a single spike 2603 that is later than the spike 2601 in the sensor signal. The delay can be due to the hydrophone being further away from the tip of the optical fiber than the distance from the acoustic detector to the tip of the optical fiber in the malaria sensor. The single spike 2603 in the hydrophone signal is followed by a generally flat baseline.

Validation of nanobubble detection with the malaria probe (such as shown in FIG. 27F) was completed by simultaneous co-registration of single nanobubbles in the optically-absorbing water model with the malaria probe and the reference 1.0 mm hydrophone. In this model, iodine was added to water to increase optical absorbance to the level to produce a background signal (FIG. 27B) and, with undiluted iodine nanoparticles in solution, to generate vapor nanobubbles (FIG. 27F). The expansion (first) spikes of nanobubbles 2601, 2701 were not temporally resolved and merged with the background spike. However, the nanobubble caused a slight shift of the background signal to the right. The nanobubble collapse stage (second spikes) 2704 were identified through the reference signal of the hydrophone. The malaria probe signal delivered the second spikes as small bumps over a distorted baseline. The distorted, non-flat baseline coupled with the limited temporal resolution appears to be an acoustic limitation/difference of the malaria probe compared to a reference hydrophone.

FIGS. 27C-27E illustrate example bulk signals from the example malaria sensor in healthy human subjects. The bulk signals are signals due to the thermal bulk response of the human tissue to the laser pulses. The bulk signals measured in healthy human subjects are consistent with the sensor signals in the bulk model as illustrated in FIG. 27B. That is, the signals in FIGS. 27C-27E also include a non-flat tail baseline 2605 after the spike 2601 correlating to the thermal response of the human tissue to the laser pulses. As described above, the non-flat tail baseline 2605 can be due to the internal functions of the sensor. In FIG. 27E, the bulk signal can have a peak-to-peak that is large enough to be falsely interpreted as a malaria-positive status under the N and HI (described in greater detail below) amplitude-derived malaria diagnostic metrics. Accordingly, the peak-to-peak amplitude-derived metrics may need to be cross-checked with other types of diagnostic metrics described herein, such as the peak-time delay metric described above, and the metrics based on the second spike described below.

FIG. 27F illustrates example combined nanobubble and bulk signals from the hydrophone and the sensor, respectively, in the nanobubble and bulk model. The signal represented by the solid line is from the hydrophone and the signal represented by the broken line is from the example malaria sensor. In both the hydrophone signal and the sensor signal, the second spike 2704 of the nanobubble signal, correlating the collapse of the nanobubble, and/or a plurality of tail spikes 2708 can be identified against the background of the baseline. However, the first spike correlating to the expansion of the nanobubble may not be identifiable against the bulk signal spike 2701, 2703. For the sensor signal, the non-flat portion of the baseline can also make it difficult to identify the first spike of the nanobubble signal.

The sensor signals in the human skin model were obtained without and after adding parasites to 250 um thick dark skin (FIGS. 27G and 27H). An intact dark skin returned signals with the background first spike (as shown by the arrow in FIG. 27G) and non-flat baseline. A second nanobubble spike was shown and indicated by the arrow in FIG. 27H. The sensor signals were co-registered with the reference hydrophone signals (FIGS. 271 and 27J), which included echo signals (as indicated by the double arrows) whose time position matches the position of melanin at the skin depth in the range 50-100 um (FIG. 27I, for intact skin). The hydrophone signal of the parasite-treated human skin returned four to five spikes (FIG. 27J). The outer pair of spikes was associated with the parasites, and the “inner” pair of spikes was associated with the melanin (with time position and interval similar to those observed for intact skin in FIG. 27I). A difference between the intact and parasite-treated hydrophone signals was an increase in the time interval between the outer spikes.

Statistical analysis of the malaria sensor and hydrophone signals is described below. There was an increase in the peak-to-peak amplitude of the first spike and the time-shift in the signals from the parasite-treated skin. As shown in FIGS. 27K and 27L with 20 signals, adding parasites has shifted the spike to the right (see arrow in FIG. 27L) and has increased its peak-to-peak amplitude. There were frequent appearances of additional spikes in the time window of 1.6-2.6 us in the signals from the parasite-treated skin. The echo pattern in the parasite-treated skin has changed from a single spike to the dual spike. The N-HI metrics show statistical difference between intact and parasite-treated skin samples.

Statistical analysis of the time-shift and amplitude of the first spike as described with reference to FIGS. 27K and 27L was performed by using N-HI amplitude metrics in specific time windows (as in non-invasive field human studies in The Gambia) and with another program that detected the time position of spikes (see Table 12 below). Both programs revealed the difference in signals of intact skin and the same skin sample after adding parasites in residual concentration.

TABLE 12
Metrics of malaria sensor signals for intact and
parasite-treated dark skin samples, 3 experiments

| | | | |
|---|---|---|---|
| | Experiment 1 | | |
| | (1^{st} spike | | Experiment 3 |
| Experiment date | capped) | Experiment 2** | (new setup)** |

1.Time position of the 1^{st} spike

| | | | |
|---|---|---|---|
| Peak 1, us, intact | Not measured | 0.33 std 0.03 | 0.29 std 0.01 |
| Peak 1, us, parasites | Not measured | 0.48* std 0.01 | 0.36* std 0.02 |
| Difference (time- | Not measured | 0.15 | 0.07 |
| shift) us, | | | |

2.N-HI metrics, 0.38-1.0 us gate, Tha = 0.16 V (1^{st} spike window)

| | | | |
|---|---|---|---|
| N, intact, | Not measured | 0 | 0 |
| N, parasites | Not measured | 0.45 | 0.58 |
| HI, intact, | Not measured | 0 | 0 |
| HI, parasites | Not measured | 0.14 | 0.18 |

3.N-HI metrics, full time range Tha = 0.16 V (similar to human
studies)***

| | | | |
|---|---|---|---|
| N, intact, | Not measured | 0.15 | 0.25 |
| N, parasites | Not measured | 0.45 | 0.68 |
| HI, intact, | Not measured | 0.01 | 0.08 |
| HI, parasites | Not measured | 0.14 | 0.21 |

4.N-HI metrics, range 2.0-2.6 us, Tha = 0.01 V (2^{nd} spike window)***

| | | | |
|---|---|---|---|
| N, intact, | 0.48 | 0.45 | 0.1 |
| N, parasites | 0.72 | 0.85 | 0.3 |
| HI, intact, | 0.14 | 0.5 | 0.06 |
| HI, parasites | 0.94 | 2.42 | 0.23 |

*significantly different from the intact sample according to a two-sample t-test, p < 0.001 (this data is not available for N-HI)
**the sample preparation has been changed from leaving the stock suspension of parasites on top of the skin (as was done on Experiment 1) to leaving only residual parasites on top of the back skin surface by staining and then washing the stock suspension. The presence of many red blood cells (without parasites) on top of the skin might have influenced the acoustic signal. Thus the experiments on Experiment 2 and Experiment 3 may have a higher fidelity.

The corresponding hydrophone data revealed a similar time-shift trend (Table 13). Average values for the first spike position and interval for three independent experiments with black skin sample of 200-300 um thickness (every day a new sample was prepared, and the skin thickness variability may exceed 50 um) were calculated. The number of signals in each group was 16-20.

TABLE 13
Statistics for the hydrophone signals co-registered
with malaria sensor signals, skin model

| | Experiment | Experiment | Experiment 3 |
|---|---|---|---|
| Experiment date | 1 | 2** | (new setup)** |
| Peak 1, us, intact | 1.30 std 0.02 | 1.24 std 0.06 | 1.45 std 0.01 |
| Peak 1, us, parasites | 1.21 std 0.07* | 1.11 std 0.04* | 1.33 std 0.04 * |
| Difference us, | 0.09 | 0.12 | 0.12 |
| Interval, us, intact | Not measured | 0.23 std 0.08 | 0.11 std 0.02 |
| Interval, us, parasites | Not measured | 0.44 std 0.06* | 0.34 std 0.07* |
| Difference, us, | Not measured | 0.21 | 0.23 |

*significantly different from the intact sample according to a two-sample t-test, p < 0.001
**the sample preparation has been changed from leaving the stock suspension of parasites on top of the skin (as was done on Experiment 1) to leaving only residual parasites on top of the back skin surface by staining and then washing the stock suspension. The presence of many RBCs (without parasites) on top of the skin might have influenced the acoustic signal. Thus the experiments on Experiment 2 and Experiment 3 may have a higher fidelity.

The observed influence of parasites on the first spike was further analyzed by using HI-N signal amplitude metrics and by varying the time window where they were calculated for. In the full time range (similar to how it has been done in human studies, with the similar amplitude threshold, see Table 13), adding parasites has increased values of both N and HI by 2.5-14 fold. The values of N and HI were close to those obtained above for the analysis of the first spike only. This may indicate a visual fact that, in the full time range mode, the first spike dominates the signal. Further, in the time range from 2.0 us (where nanobubble signals were observed for the same sensor in the experimental water model of nanobubbles and where nanobubble-like signals were observed in the non-invasive human study in The Gambia) to 2.6 us (the max time range to avoid any echo signals), a 2-6 fold increase in both metrics was observed after parasites were added.

The time-shift observed for the first spike after parasites were added matches the hydrophone data for the same samples. A similar direction and value of the time shift was observed with the hydrophone for the first spike (note an opposite location of the hydrophone hence an opposite time-shift). However, the malaria sensor may not resolve melanin and parasite spikes as the hydrophone did. Instead, it appears that sensor has integrated closely located spikes into one spike with varying amplitude as adding parasites has increased the peak-to-peak amplitude of the first spike detected with the sensor but did not increase the amplitude of any spike in the hydrophone signal. These differences in signal parameters could be caused by a narrow acoustic frequency bandwidth of the sensor compared to that of the hydrophone. Nevertheless, the optimization of the time window range and the amplitude threshold allows for distinguishing intact and parasite skin even with a slow-speed sensor (see, e.g., FIG. 27M).

In the field human studies the malaria sensor returned typical signals for healthy (n=25) (FIG. 27N) and malaria-infected (n=30) subjects (FIG. 27O). FIG. 27N illustrates the malaria probe signals obtained from healthy subjects. FIGS. 27O and 27P illustrate example combined nanobubble and bulk signals in malaria-positive human subjects using the malaria sensor. In malaria-positive subjects, the peak-to-peak amplitude of the first spike increased while the peak position shifted slightly to the right, by about +50±47 ns (statistically significant, p=0.0001), as indicated by a horizontal arrow. The analysis was based on the Gambia 2017 data for African type of dark skin and Plasmodium Falciparum type of malaria parasites. Similar to the sensor signal in the nanobubble and bulk model, a plurality of small second spikes or tail spikes 2708 correlating to nanobubble generation can be observed. In the illustrated example, the tail spikes 2708 can be observed in the time window about 2 s after the delivery of the laser pulse. Similarly, the first spike of the nanobubble signal in FIGS. 27O and 27P coincides with the spike 2701 of the bulk signal and may not be identifiable against the background of the bulk signal and/or the non-flat baseline after the spike. However, the tails spikes 2708 are not accounted for in the peak-to-peak amplitude-derived diagnostic metrics because the amplitudes of the tail spikes 2708 are lower than the maximum peak-to-peak amplitude, which can depend on the amplitude of the spike 2701 of the bulk signal. Statistical analysis of the above differences used the signal amplitude HI and N signal metrics (Table 15 and FIGS. 28C-28E below).

Accordingly, new diagnostic metrics that can account for the second spike correlating to the collapse of the nanobubble may be needed. FIG. 28A illustrates the new metrics. N nb is the number of second spikes per run of N (for example, 60) signals. N nb can represent the number of transient vapor nanobubbles detected in a specific time window. The time window can be from about 2 s to about 5 s. T nb is the time interval between the first and second spikes in the combined signal. T nb can represent or approximate the lifetime of a nanobubble and can be a measure of the maximal size of a nanobubble. Table 14A below summarizes HZ (HEMOZOIN) (hemozoin-positive) signals in blood samples of cultured P. Falciparum parasites. The results are also shown in FIG. 28B. In the blood P. Falciparum model in flow test, HI increases with the size of HZ (HEMOZOIN) nanoparticle cluster (in a parasite or if the blood is free from parasites. Gametocytes generate larger nanobubbles since their level of HZ (HEMOZOIN) is higher. Table 14B summarizes HZ (HEMOZOIN) (hemozoin-positive) signals in blood samples of positive human subjects. The presence of hemozoin in skin can be observed by hemozoin-generated vapor nanobubble signals in human skin and malaria-positive mosquitoes fed from human blood

TABLE 14A
HZ (hemozoin) signals in blood samples of cultured Plasmodium
Falciparum parasites, 10 Par/uL, The malaria model with
Plasmodium Falciparum-infected human blood

| Parasite Type | 36 hour trophozoa | Gametocytes |
|---|---|---|
| Blood HI | 0.37 | 4.8 |

TABLE 14B
HZ (hemozoin) signals in blood samples
of positive human subjects

| | | |
|---|---|---|
| Gametocyte count in blood | 0-1 | >30 |
| Blood HI | 0.33 | 2.24 |

The HI-N amplitudes in FIGS. 28C-28E and Table 15 below were analyzed for the time window covering the whole signal and only the first spike and the time window for the second spike.

TABLE 15
HI-N metrics for the Gambia (Plasmodium Falciparum malaria study)
field human data, obtained for the full time window of the signal

| Amplitude | | | 1^{st} spike, | 2^{nd} spike analysis, | |
|---|---|---|---|---|---|
| metrics, | | | Time window | Time window | |
| an ankle | Full time-window | | 1.02-5.0 us | 2.0-5.0 us | |
| N, healthy | 0.03 | | 0.011 | 0.01 | |
| N, malaria | 0.34 | (×11)* | 0.139 (×13)* | 0.07 | (×7)* |
| HI, healthy | 0.007 | | 0.002 | 0.004 | |
| HI, malaria | 0.110 | (×15)* | 0.041 (×21)* | 0.037 | (×10)* |

The N-HI diagram (for all subjects, single data point corresponds to a single subject) shows the following statistical properties of signals. The metrics were calculated for the similar settings for the time window to include both the first spike and secondary spikes and an amplitude threshold of 0.16 V. Table 15 shows group-averaged values (N=30 for malaria, N=25 for healthy). For human field data, one specific location, an ankle, was analyzed. When assuming a time-shift to the right of the first spike and setting the time-window at 1.02 us to 5.0 us, the amplitude threshold unchanged Tₕₐ=0.16 V, the separation of healthy and malaria data improves, their group averages increasing by more than one order of magnitude and two groups being statistically significantly different (p<0.001). Metrics of the first spike, with the time window optimized for the time-shift observed, provided the best diagnostic separation for signals obtained with the slow-speed sensor.

When applied to the time window associated with the second spike of a nanobubble collapse, at time window 2.0-5.0 us, only peak-to-peak amplitudes (without subtracting a non-flat baseline) were analyzed. This limitation of the analysis decreased the separation between healthy and malaria-positives but still indicated some statistically significant difference between these subject groups. The distribution of HI-N data in the model (FIG. 27M) and humans (FIGS. 28C-28E) follows the same trend.

In the skin model experiment, the incidence of the second spike was lower than in field human data. This can be explained by two factors: (1) mechanical properties of live (humans) skin are more favorable for the collapse of nanobubble compared to properties of dead skin (the model), and (2) real human skin had more parasites and they also were closer to the surface compared to the condition of residual single parasites at 250 um depth in the model.

FIG. 29A illustrates the T nb-N nb diagram for the Plasmodium Falciparum malaria using the signal obtained from the Stage 1 study in The Gambia, where N is number of second spikes nanobubbles observed, T is time interval between the first and second spikes (the nanobubble lifetime, which is proportional to the maximal diameter of the nanobubble). The N nb and T_{nb} values are subject-averaged per a test run of 60 laser pulses. The diagonal line represents a malaria diagnostic threshold, with the T nb-N nb value above the line being interpreted as a malaria-positive status and the T nb-N nb values below the line being interpreted as a malaria-negative status. The incidence and interval for the secondary spike was additionally analyzed with time-based metrics and the probability of observation of the spike (FIG. 29A). Compared to the amplitude analysis of the second spikes (FIG. 28C), the time-based approach in FIG. 29A can be more efficient. Compared to the first spike, not all malaria-positive signals included the second spikes. This deficiency of the second spike was similar to that observed in the human skin model and may be associated with the mechanical properties of the skin, plastic deformation during the expansion of nanobubble and viscous losses that reduces the nanobubble energy and prevent its collapse as it occurs in the water, a fully elastic medium without plastic deformations. Nevertheless, the second spike data from the two field studies of two malaria strains, Plasmodium Falciparum and Plasmodium Vivax, in two groups, clinical and asymptomatic (the latter was studied via the mass screening) show the diagnostic significance of the second spikes in signals even obtained with a slow-speed sensor.

FIG. 29B illustrates the N nb histogram for the Plasmodium Falciparum malaria using the signals obtained in the study groups in The Gambia. FIG. 29C illustrates the N nb histogram for the Plasmodium Vivax malaria using the signals obtained in the study group in Sumatra. The vertical line represents the diagnostic threshold, which can be set at an Nnb value of 5 per a test run of 60 laser pulses.

Table 16 below summarizes the group-averaged N nb values and additional details of FIGS. 29B and 29C.

TABLE 16
Group-averaged occurrence of 2nd spikes among 60 ankle signals
in human subjects in two field studies
Nnb: group-averaged values and two-sample test (P < 0.005
means two groups are significantly different)

| | | | P, | | P, healthy- |
|---|---|---|---|---|---|
| | | | health- | | positive, |
| | | | positive, | Positive, | Mass |
| | | Positive, | clinical | Mass | Screening |
| Study Group | Healthy | Clinical | (Clin) | Screening | (Asym) |
| P. | 1.8 ± 2.0 | 11.9 ± 6.1 | 5.4E−7 | 9.6 + 6.7 | 8.9E−5 |
| Falciparum, | | | | | |
| The Gambia | | | | | |
| P. Vivax, | 2.3 ± 4.1 | 10.0 ± 3.4 | 2.6E−4 | — | — |
| Sumatra | | | | | |

Several factors influenced the occurrence of the second spike: (1) not all nanobubbles collapsed, not all collapsed nanobubbles were detected, only those with the lifetime above 1 us were detected, because the second spikes of smaller nanobubbles were obscured by a non-flat baseline of the signal output of malaria sensor. The occurrence of collapse spikes (2^{nd} spikes) was random through the test run from 1st to 60st signals. They were not linked to specific, for example, initial laser pulses like they were in the skin model where a strong signal decay was observed. This difference between the dead and live skin suggests that the laser-exposed volume in live skin does not remain static during the test (which can be about 3 seconds) and new hemozoin targets can enter the volume during the test. This may be the result of plastic deformation of skin or cracks induced by nanobubbles and the resulting “mixing” of the skin volume.

In summary, in several independent experiments, adding parasites (including the residual levels of parasites) to the bottom of the human dark skin sample of 200-300 um thickness has caused changes in the time position and peak-to-peak amplitude of the first spike and the appearance of irregular second spikes in signals of the slow-speed sensor. Co-registration of acoustic signals in the skin sample with two sensors, the reference hydrophone and the malaria sensor, presented independently obtained evidence of the signals associated only with parasites in skin. Unlike the hydrophone, the malaria sensor does not resolve signal spikes which are close in time. Instead, it reveals some effects of the integration of close spikes. These effects influence time and amplitude parameters of the first, largest, spike in the signal, whose amplitude and time position correlate to parasites in the skin. The similarity of signal shapes and signal metrics observed in three different studies-non-invasive human studies, water model of nanobubbles, and the skin model with human parasites —suggests that the non-invasive skin signals detected in the field in malaria-positive subjects were caused by vapor nanobubbles generated around parasites in the skin.

Metrics N nb and T nb are independent of the amplitude derived metrics described above, and can be used in addition to and/or instead of the amplitude-derived metrics. In some embodiments, the malaria sensor can have reduced distortions of the tail baseline or a flat tail baseline to make it easier to detect the second and/or tail spikes.

Optical excitation and acoustic detection of parasites in a liquid sample of whole blood will now be described. The liquid sample included water, human blood (whole) and human blood with 50 Plasmodium Falciparum parasites per microliter (the lower limit of the microscopy detection of parasites in blood). Static and flowing samples were studied in an Eppendorf tube. The flow was achieved by the pipette-induced mixing of the tube content during the signal collection.

The same laser pulse as above (220 ps, 671 nm, 15 μJ) was delivered via the optical fiber (with 105 or 50 μm fiber core) as a 60-pulse train. The 50-um fiber was used with a reduced laser pulse energy of 3.6 uJ in order to maintain the same optical fluence as the one at the exit of the 105-um fiber. Acoustic detection was performed using a reference hydrophone of 1.0 mm diameter (with a bandwidth of 10-12 MHZ (hemozoin), and a sensitivity of 0.5 V/MPa at 5 MHZ (hemozoin)). The hydrophone tip was located at 2 mm (approximately) distance from the source.

The water and blood signals were detected as a reference (FIGS. 30C and 30D). As shown in FIG. 30D, residual absorbance by whole blood components (such as hemoglobin and other proteins) resulted in highly reproducible single bipolar spike. This spike reported both a compression and tensile pressure components and represented typical thermo-elastic signal in response of optically absorbing volume as determined by the laser beam aperture and penetration depth to a single laser pulse. The reproducibility and noise floor can be seen in the overlay of 60 signals obtained in one test. The flow of the blood did not seem to induce detectable changes into the intact blood signal, which has been considered as the background (see FIG. 30E). Both in static and flowing blood samples, a second spike was occasionally observed (a small signal in the time window 2-4 us in FIGS. 30D and 30E) with the probability of less than 1%. This second spike may not indicate a nanobubble because a nanobubble of such a large lifetime would have produced a second spike of a much higher amplitude.

As shown in FIGS. 30F and 30G, adding Plasmodium Falciparum malaria parasites has returned signals with typical vapor nanobubble signals with second spikes and sometimes time-resolved first spikes as compared to nanobubbles in the water model disclosed herein. The signals are different from the background spike of the whole blood. As shown in FIG. 30G, the incidence of nanobubble signals has increased in the flowing sample. The amplitude of the second spike has increased with the nanobubble lifetime, which was observed in the range from 0.5 us (smallest nanobubbles detected) to 2.7 us. Both the first and second nanobubble spikes had mainly positive components, without pronounced negative components. This may indicate (1) a low elastic modulus of the sample (in line with water-like mechanical properties of the blood), and (2) absence of significant tensile stress compared to those observed in the skin model. Under an identical parasite density, the static sample (FIG. 30F) returned fewer and smaller sized nanobubbles than the flowing sample (FIG. 30G) in response to a 60 laser pulse train. Static sample generated nanobubbles in response to only 1-2 first laser pulses, thus confirming the destruction of HZ (hemozoin) cluster after it generated initial nanobubble(s). Since the size of HZ (hemozoin) cluster varies from parasite to parasite, mixing the laser-exposed volume brought more parasites for the optical excitation and hence resulted in an increase in the number of nanobubble signals and in their lifetime. A time interval between the background and the first nanobubble spikes was often observed in FIG. 30G. The background signal was technically generated at the hydrophone-blood interface while the nanobubbles were generated at the whole laser pulse penetration depth and thus might have emerged at some distance from the hydrophone tip. The maximum time-interval observed corresponded to the distance of about 300-350 um. The amplitude of the second spike was close to that of the first spike (when the first spike was time-resolved), which may be another piece of evidence of an unrestricted collapse of a nanobubble in the whole blood.

As shown in FIGS. 30H and 301, reducing the laser-exposed volume by replacing the 105 um core fiber with the 50 um core fiber has resulted in a much lower background signal (FIG. 30H). At the same time, fewer nanobubbles were generated/detected (FIG. 32, right) compared to the excitation via the 105 um core fiber (see also Table 17 with statistical analysis).

TABLE 17
Statistical HI-N (amplitude parameters) analysis of
the influence of the optical excitation volume on the
background and parasite signals in whole blood (full
time window, amplitude of both spikes considered)

| | | Nanobubble | Nanobubble |
|---|---|---|---|
| | Peak-to-peak amplitude | signal in | signal |
| Optical fiber | of the background signal, | Plasmodium | Plasmodium |
| core diameter, | mV (after subtracting the | Falciparum | Falciparum |
| um | noise level) | blood, N | blood, HI |
| 105 | 7.9 (7.2) | 0.24 | 0.29 |
| 50 | 2.5 (0.6) | 0.061 | 0.064 |

Comparing the signals in FIGS. 30F and 30G to skin signals obtained under similar conditions, parasites in the whole blood apparently do not restrict the nanobubble expansion and do not dampen the nanobubble collapse the way it was observed in skin. This is in line with much higher elasticity and lower viscosity of the blood than the skin. Thus, the conditions of blood (micro) vessels are more favorable for the detection of parasites compared to those when HZ (hemozoin) or the parasite is surrounded by the skin only and without any adjacent liquid.

In the flowing sample, the amplitude HI-N metrics, coupled with tailored time-window (1.25us) which excluded the background signal, and the amplitude threshold of 4 mV, resulted in zero HI and N values for intact blood and 0.29 and 0.24, respectively, for blood with Plasmodium Falciparum parasites at the density 50 p/uL.

The volume exposed to the laser pulse may influence the background signal metrics. A smaller fiber has resulted in a reduction of 4 to 12-fold in the amplitude of the background signal (see, e.g., FIGS. 30H and 30I). Further, the ratio of the amplitude of the first nanobubble spike to that of the background signal has increased. A smaller excitation volume improves the nanobubble detection but covers fewer parasites with a single laser pulse. The parameter N has dropped by 4-fold so 4-5 times more locations/volumes must be probed with the small diameter laser pulse in order to achieve the same number of parasites detected as by a laser pulse delivered via the 105 um core fiber. However, an advantage of a smaller excitation volume is a lower laser energy required, 4-fold lower in this case.

In these experiments, one parasite density has been used, 50 p/uL. For water-diluted blood samples (with a factor of 20:1), the threshold of detection of the same Plasmodium Falciparum parasites was 0.01 parasite per microliter of suspension. This is equal to 0.2 p/uL in the whole blood, and is still below the detection threshold of regular PCR, microscopy, and RDT, the three standard methods for detecting parasites in blood in the clinical and laboratory settings.

Compared to the skin model, the human whole blood seems to better support the generation of vapor nanobubbles without damping their expansion and collapse, similar to those in water. Thus, non-invasive detection of parasites in skin may benefit from the presence of blood around the subcutaneous parasites, which is the case for substantially all micro-vessels in dermis, the smallest of which are 5-10 um in diameter. The smallest nanobubbles detected in the human whole blood had a lifetime of around 500 ns, which is close to the detection threshold of 300-400 ns lifetime in the water model. Reducing the laser-probed (exposed to the pump laser pulse) volume may improve the signal-to-background ratio in detecting parasite-generated nanobubbles, but may require increasing the number of probed (scanned) locations and hence the diagnostic time. In addition, diluted blood samples returned more nanobubbles than the whole blood due to deeper laser penetration.

A chicken breast model was used to analyze parasites in melanin-free tissue. The sample was prepared from manufactured chicken breast meat (which remained visually wet). To model the malaria infection, blood with parasites was injected with a needle to the depth of 0.5-1.0 mm (as was verified later by measuring a cross-section of the sample as shown in FIG. 31A). A thick layer of meat underneath served as acoustic damping medium. The local density of parasites in tissue was estimated to be in the range 10-100 p/uL based upon the initial injection concentration of 250 p/uL, with an assumption of limited diffusion into the volume 1.5 to 3 times larger than the injected volume. Optical excitation and acoustic detection were identical to those in the whole blood model and used 1.0 mm hydrophone at 2 mm distance from the tissue surface (FIG. 31B). 60 laser pulses were applied to each location and ultrasonic gel was used as a coupling medium.

As shown in FIG. 31C, signals observed revealed none or very small background (bulk) signal in intact tissue. This was different from both the blood (with hemoglobin creating the volume background opto-acoustic signal) and dark skin (with melanin creating the volume background opto-acoustic signal) models. Adding the parasites to the depth of 0.5-1.0 mm resulted in two-spike signals typical for vapor nanobubbles and similar to those detected in blood or water (FIG. 31C). These signals had no negative components, thus indicating no tensile stress unlike signals detected in the human skin model. The second spikes were always observed when the first spikes were detected. The amplitude of the second spike was close to that of the first spike. These features indicated the generation of nanobubbles in an elastic medium without plastic deformation, without viscous losses to nanobubbles during their expansion, and without damping of the nanobubble collapse. This result is in contrast to the nanobubble generation in the human skin model where viscous losses during the expansion and damping of the collapse were usually observed.

The other difference to the tissue model describe above was that nanobubble signals in this model were not always observed in response to the first laser pulse and sometimes were detected in the same location after tens of laser pulses (FIG. 31D). This effect was similar to that observed in the flowing blood sample described above. It appears that the probed volume in the tissue included some liquid component which has been actively mixed during the laser exposure. Such mixing was not caused by the first nanobubble but might have been caused by the ongoing re-distribution of the injected parasite blood. Statistical signal amplitude metrics N and HI were obtained to quantify the difference between the samples, as shown in Table 18 below.

TABLE 18
The signal amplitude metrics N and HI obtained for the full
time window for intact and parasite-injected tissue

| Sample | N | HI |
|---|---|---|
| Tissue + Blood (n = 8) | 0.015 | 0.001 |
| Tissue + Plasmodium | 0.15* | 0.16* |
| Falciparum − Blood (n = 10) | | |

*the group was found to be statistically significantly different from the control in two-sample t-test

Despite a much higher tissue depth, 500-1000 um in the chicken breast model compared to 250 um in the human dark skin model, nanobubble signals were stably detected in melanin-free chicken breast tissue. Therefore, the presence of melanin and associated opto-acoustic background appears to be a limiting factor in the detection of parasite-generated vapor nanobubbles in human skin. As shown in FIG. 31C, another and independent feature of this model was the absence of tensile stress, plastic deformation and high elastic modulus of the tissue (compared to those of the skin model) resulting in the unrestricted expansion and collapse of vapor nanobubbles around malaria parasites. Absence of the high background signal coupled with unrestricted nanobubble behavior provided strong signal with better (10 to 100-fold) separation of the signal amplitude metrics of intact and “infected” tissues (Table 18), compared to the human skin model (Table 10). The result for this model suggests that properties of the tissue, for example, stiffness (elastic modulus), plastic deformation, and viscosity may influence the diagnostic sensitivity of the method, which appears to be higher in tissue with properties similar to those of water, that is, lower stiffness, viscosity and plastic deformation.

In the tissue model with chicken breast and the injected human blood, malaria parasites generated vapor nanobubbles similar to those generated in the gold-water model, with symmetrical expansion and collapse and without damping effects observed in the human skin sample. Nanobubble signals were observed at parasites at a tissue depth of up to 1.0 mm. The difference between the chicken breast and human skin models can be explained by the differences in the mechanical properties of two tissue samples and, additionally, by the higher volume of liquid (blood around parasites) in the chicken breast model compared to that in human skin model.

FIGS. 32A-32C illustrate additional ultrasonic signals from a single transient vapor nanobubble in the gold-water model of a single vapor nanobubble (32A), in the gold—human skin model (32B), and around malaria parasites in human skin (32C). In the gold-water model, the acoustic signals report an unrestricted and symmetrical expansion and collapse of a transient vapor nanobubble. Symmetry is seen through similar amplitudes of the first (expansion) and second (collapse) spikes. In the gold-skin model, the acoustic signals report much faster expansion (although restricted, the amplitudes of both acoustic and optical signals are lower than those in water) and delayed (compromised) collapse, which results in a lower relative amplitude of the second spike. This delayed collapse is caused by plastic (vs elastic in water) deformations and possible formation of micro-voids. In the skin with parasite model, the acoustic signals appear to be similar to the previous case of vapor nanobubble in skin, with restricted expansion and delayed collapse of the nanobubble. The damping effect of skin can also be seen in FIG. 26P. The comparison of the three different cases studied with two different methods shows a good agreement in skin signals for nanobubbles and parasites, and thus the vapor nanobubble nature of the parasite signals in skin.

In summary, malaria parasites were detected through the acoustic response of vapor nanobubbles (defined earlier as hemozoin-generated vapor nanobubble). Hemozoin-generated vapor nanobubbles were generated and detected in whole blood, human dark skin and chicken breast tissue models, and in human subjects with Plasmodium falciparum and Plasmodium vivax parasite strains in the field studies. The result was achieved through comparative studies in six different experimental systems (in addition to two previously studied systems, individual infected human red blood cells and infected animals).

For the liquid sample, the method detects down to 0.01 Plasmodium Falciparum parasite (gametocyte) per microliter of solution and 0.1 Plasmodium Falciparum parasite (trophozoa) per microliter of solution. For the dark human skin, the method detected residual single Plasmodium Falciparum parasites (gametocytes). For the non-invasive model with a dark skin sample, the method might not detect all parasites present in the skin because it could detect only relatively large nanobubbles with the lifetime above 0.7 us. Such a high detection threshold suggests that smaller vapor nanobubbles are being generated in the skin but cannot be detected with the current excitation and detection setups.

The human field and two skin model studies revealed the influence of the mechanical properties of skin, such as the high elastic modulus and viscosity, and plastic deformation, on the generation and collapse, and hence the detectability of (relatively large, around 10 um max diameter) vapor nanobubbles. Smaller nanobubbles may be less influenced by the above mentioned properties of the skin and hence may improve the detection of parasites.

In human dark skin, the background signal of melanin may have a 10-fold higher amplitude than that of a parasite-generated nanobubble. The suppression of the melanin signal and its separation (decoupling) from the nanobubble signal improve the diagnostic performance of non-invasive skin-based device.

Another approach to improve the diagnostic performance is to use a minimally invasive skin probing device with the optical fiber penetrating 200 um of the upper skin may suppress the melanin background and improve the optical excitation of nanobubbles.

### Terminology

Terms of orientation used herein, such as “proximal,” “distal,” “radial,” “central,” “longitudinal,” and “end” are used in the context of the illustrated embodiment. However, the present disclosure should not be limited to the illustrated orientation. Indeed, other orientations are possible and are within the scope of this disclosure. Terms relating to circular shapes as used herein, such as diameter or radius, should be understood not to require perfect circular structures, but rather should be applied to any suitable structure with a cross-sectional region that can be measured from side-to-side. Terms relating to shapes generally, such as “circular” or “spherical” or “semi-circular” or “hemisphere” or any related or similar terms, are not required to conform strictly to the mathematical definitions of circles or spheres or other structures, but can encompass structures that are reasonably close approximations.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

The terms “approximately,” “about,” and “substantially” as used herein represent an amount close to the stated amount that still performs a desired function or achieves a desired result. For example, in some embodiments, as the context may permit, the terms “approximately”, “about”, and “substantially” may refer to an amount that is within less than or equal to 10% of the stated amount. The term “generally” as used herein represents a value, amount, or characteristic that predominantly includes or tends toward a particular value, amount, or characteristic. As an example, in certain embodiments, as the context may permit, the term “generally parallel” can refer to something that departs from exactly parallel by less than or equal to 15 degrees.

While a number of variations of the disclosure have been shown and described in detail, other modifications, which are within the scope of this disclosure, will be readily apparent to those of skill in the art based upon this disclosure. It is also contemplated that various combinations or sub-combinations of the specific features and aspects of the embodiments may be made and still fall within the scope of the disclosure. Accordingly, it should be understood that various features and aspects of the disclosed embodiments can be combined with or substituted for one another in order to form varying modes of the disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings) may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination so disclosed.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Some embodiments have been described in connection with the accompanying drawings. The figures are not drawn to scale where appropriate, but such scale should not be limiting, since dimensions and proportions other than what are shown are contemplated and are within the scope of the disclosed invention. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various embodiments can be used in all other embodiments set forth herein. Additionally, any methods described herein may be practiced using any device suitable for performing the recited steps.

Although this invention has been disclosed in the context of certain embodiments and examples, the scope of this disclosure extends beyond the specifically disclosed embodiments to other alternative embodiments and/or uses of the invention and obvious modifications and equivalents thereof. Any system, method, and device described in this application can include any combination of the preceding features described in this and other paragraphs, among other features and combinations described herein, including features and combinations described in subsequent paragraphs. While several variations of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. Various features and aspects of the disclosed embodiments can be combined with, or substituted for, one another in order to form varying modes of the disclosed invention. Thus, it is intended that the scope of the present invention herein disclosed should not be limited by the particular disclosed embodiments described above, but should be determined only by a fair reading of the claims that follow.

What is claimed is:

1. An apparatus configured for diagnosing malaria noninvasively, the apparatus comprises:
a sensor probe having a probe body terminating at a probe tip surface, the probe tip surface configured to be placed into contact with a predetermined detection location;
an optical source configured to generate a plurality of laser pulses of at least one predetermined energy level or at least one predetermined wavelength, the optical source comprising an optical fiber terminating at or near the probe tip surface, the plurality of laser pulses configured to cause generation of one or more transient vapor nanobubbles around malaria-specific nanoparticles at the predetermined detection location; and
an acoustic detector configured to detect acoustic pulses generated by the one or more transient vapor nanobubbles and output one or more signals indicative of the detected acoustic pulses to at least one processor, the acoustic detector comprising a piezo element and being flat, the one or more acoustic detectors comprising an opening configured to accommodate a tip of the optical fiber such that the optical fiber is centered relative to the acoustic detector, wherein the optical fiber extends at least to a distalmost surface of the acoustic detector,
wherein the optical source and the acoustic detector are enclosed within the probe body.

2. The apparatus of claim 1, wherein the optical source comprises two or more optical fibers, wherein each of the two or more optical fibers is located between two acoustic detectors.

3. The apparatus of claim 1, wherein the optical fiber has a core diameter of about 50 μm to about 200 μm.

4. The apparatus of claim 3, wherein the optical fiber has a core diameter of about 100 μm.

5. The apparatus of claim 1, wherein the optical source further comprises a laser pulse generator coupled to the optical fiber.

6. The apparatus of claim 5, wherein the laser pulse generator is configured to generate laser pulses of same or different energy levels and/or wavelengths.

7. The apparatus of claim 1, wherein the acoustic detector comprises two or more piezo elements configured to detect signals of same or different frequency spectra.

8. The apparatus of claim 1, wherein the piezo element comprises a navy type II or type VI material or a composite material.

9. The apparatus of claim 1, wherein a tissue-facing surface of the acoustic detector is about 0.1 mm to about 0.3 mm recessed from the probe tip surface.

10. The apparatus of claim 9, further comprising a front layer between the probe tip surface and a tissue-facing surface of the acoustic detector.

11. The apparatus of claim 1, wherein an outer wall of the optical fiber is separated from a radially inner edge of the acoustic detector by about 0.01 mm to about 0.03 mm.

12. The apparatus of claim 1, wherein an outer surface of the optical source is separated from a radially outer edge of the acoustic detector by about 0.3 mm to about 1.5 mm.

13. The apparatus of claim 1, wherein the sensor probe is reusable.

14. The apparatus of claim 1, wherein the sensor probe further comprises a disposable cap.

15. The apparatus of claim 1, wherein the plurality of laser pulses are configured to cause generation of nanobubbles around malaria-specific nanoparticles in blood and/or tissue.

16. The apparatus of claim 1, wherein the predetermined detection location is a patient's skin at the patient's wrist, ankle, lip, or tongue base.

17. The apparatus of claim 1, wherein the probe tip surface is configured to be covered with a layer of gel before being placed into contact with the predetermined detection location.

18. The apparatus of claim 1, further comprising a housing, wherein the probe body is at least partially disposed within the housing; and a spring disposed between a proximal end of the housing and the proximal end of the probe body, the spring biasing the probe body toward a distal end of the housing.

19. The apparatus of claim 1, wherein a distance between an outer wall of the optical source and a radially inner edge of the one or more acoustic detectors, R1, is 0.01 mm to 0.03 mm.

20. The apparatus of claim 19, wherein the R1 is 0.01 mm to 0.03 mm such that the acoustic pulses strike a flat surface of the one or more acoustic detectors at an angle of incidence, α, of less than 45° so as to improve a signal strength of the acoustic pulses striking the flat surface of the one or more acoustic detectors.
